(19) United States
(12) Patent Application Publication
(10) Pub. No.: US 2010/0029654 A1
(43) Pub. Date: Feb. 4, 2010

(54) CARDIOVASCULAR COMPOSITIONS AND USE OF THE SAME FOR THE TREATMENT OF ALZHEIMER'S DISEASE

(75) Inventor: Giulio Pasinetti, New York, NY (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: MOUNT SINAI SCHOOL OF MEDICINE

(21) Appl. No.: 12/294,080

(22) PCT Filed: Mar. 22, 2007
(86) PCT No.: PCT/US2007/064718

§ 371 Date: Sep. 11, 2009

Related U.S. Application Data
(60) Provisional application No. 60/785,013, filed on Mar. 23, 2006.

(51) Int. Cl. A61K 31/41; A61K 31/496; A61K 31/4375; A61K 31/135; A61K 31/165; A61K 31/403; A61K 31/44; A61K 31/502; A61K 31/4164; A61K 31/47; A61K 31/343; A61K 31/519; A61P 9/00

(52) U.S. Cl. 514/248; 514/252.17; 514/280; 514/651; 514/620; 514/411; 514/356; 514/381; 514/401; 514/307; 514/469; 514/262.1

(57) ABSTRACT

Methods and compositions for the treatment of Alzheimer's Disease are described. More specifically, the invention demonstrates that administration of cardiovascular agents to a mammal suffering from the symptoms of Alzheimer's Disease causes an amelioration of those symptoms. The finding of the present invention can be used in treatment regimens designed to attenuate or prevent Alzheimer's Disease.

The present application claims the benefit of priority of U.S. Provisional Patent Application Ser. No. 60/785,013 which was filed Mar. 23, 2006 and is incorporated herein by reference in its entirety.

### FIELD OF THE INVENTION

The present invention relates to methods and compositions for use in the treatment of Alzheimer's Disease. More particularly, it is based on the discovery that administration of cardiovascular agents to a mammal that exhibits symptoms of Alzheimer's Disease is effective to attenuate, ameliorate or even prevent Alzheimer's Disease.

### BACKGROUND OF THE INVENTION

Alzheimer's disease is characterized by the abnormal deposition of amyloid in the brain in the form of extra-cellular plaques and intra-cellular neurofibrillary tangles. The rate of amyloid accumulation is a combination of the rates of formation, aggregation and egress from the brain. It is generally accepted that the main constituent of amyloid plaques is the 4 kD amyloid protein (βA4, also referred to as Aβ, β-protein and βAP). This protein is formed as a result of proteolytic processing of a precursor protein of much larger size, the amyloid precursor protein (APP or AβPP), which has a receptor-like structure with a large ectodomain, a membrane spanning region and a short cytoplasmic tail. The Aβ domain encompasses parts of both extra-cellular and transmembrane domains of APP, thus its release implies the existence of two distinct proteolytic events to generate its NH₂— and COOH— termini. At least two secretory mechanisms exist which release APP from the membrane and generate soluble, COOH-truncated forms of APP (APPₛ). Proteases that release APP and its fragments from the membrane are termed “secretases.” It is now recognized that most APPₛ is released as a result of α-secretase which cleaves within the Aβ protein to release α-APPₛ and precludes the release of intact Aα. A minor portion of APP.sub.s is released by a β-secretase (“β-secretase”), which cleaves near the NH₂-terminus of APP and produces COOH-terminal fragments (CTFs) which contain the whole aβ domain.

It is the activity of β-secretase or β-site amyloid precursor protein-cleaving enzyme (“BACE”) that is believed to generate the abnormal cleavage of APP, production of Aβ, and accumulation of β amyloid plaques in the brain, which is characteristic of Alzheimer's disease (see R. N. Rosenberg, Arch. Neurol., vol. 59, September 2002, pp. 1367-1368; H. Fukumoto et al, Arch. Neurol., vol. 59, September 2002, pp. 1381-1389; J. T. Huse et al, J. Biol. Chem., vol 277, No. 18, issue of May 3, 2002, pp. 16278-16284; K. C. Chen and W. J. Howe, Biochem. Biophys. Res. Comm, vol. 292, pp 702-708, 2002).

BACE is a type I membrane-associated aspartic protease (Sinha et al., 1999; Vassar et al., 1999; Yan et al., 1999). It produces a C99 APP cleavage product that is the immediate precursor of amyloid-β. The C99 product is further cleaved to produce the 40 to 42 amino acid Aβ peptide in the brain, which is deposited as extracellular insoluble aggregates in brain tissue (Glenner and Wong, Biochem. Biophys. Res. Commun. 120:885-890 (1984); Masters et al., EMBO J. 4:2757-2763 (1985)).

It has long been established that hypertension can lead to vascular dementia (Roman, 2005). Recently, evidence has demonstrated that blood pressure may be also a risk factor for AD (Luchsinger and Mayeux, 2004). However, the challenge in linking AD to hypertension stems from the fact that there is a lengthy period between the initiation of AD, and the appearance of symptoms. Moreover, in patients with AD, synaptic disconnection of the autonomic brain nuclei, as well as physical immobilization often lead to a paradoxical fall in blood pressure (reviewed in Staessen and Birkenhager, 2004). Cross-sectional studies therefore, cannot disclose the true nature of the relation between dementia and blood pressure. However, despite this apparent complexity making difficult to interpret the relationship between hypertension and AD, there has been some recent speculation that certain cardiovascular drugs with antihypertensive properties may decrease the incidence of AD (Forrette et al, 2002; Lopez-Arrieta and Birke, 2002; Guo et al, 1999).

For example, in the Cochrane Dementia and Cognitive Improve Improvement Group's Specialized Register, which contains reports of trails from all major medical databases, Lopez-Arrieta and Birke (2002) found that the Ca⁺⁺ receptor antagonist nimodipine, often used in cerebrovascular disorders, may decrease the incidence of AD in cases with hypertension. In addition, Guo et al (1999) reported that the use of certain cardiovascular agents, in particular β-blockers or Ca⁺⁺ receptor antagonists, protected elderly hypertensive subjects from developing AD (Lopez-Arrieta and Birke, 2002; Guo et al, 1999). Most interestingly, the double-blind placebo-controlled Syst-Eur trial stands out as the only study of antihypertensive agents which, after a median follow-up of two years, has demonstrated a 50% reduction in the incidence of all types of dementia primarily AD in eligible hypertensive cases (Forrette et al, 2002). Interestingly, the main component of the active treatment in the Syst-Eur study was the Ca⁺⁺ channel blocker is nitrendipine, which interestingly, is one of most potent Aβ₁₋₄₂ lowering antihypertensive agent identified in high-throughput drug screenings.

Despite these encouraging reports, other studies failed to support the efficacy of antihypertensive agents in AD dementia. For example, the Rotterdam study reported that the use of antihypertensives did not significantly affect the relative risk for AD among 7046 elderly who were free of dementia at baseline (in't Veld et al., 2001). Thus, there is presently little consensus on whether antihypertensive drugs are useful in decreasing the incidence of AD in hypertensive cases.

As noted above, the rate of amyloid accumulation in the brain is a combination of the rates of formation, aggregation and egress from the brain, wherein there is an abnormal accumulation of the Aβ peptide. Any agent that decreases the rate of formation or aggregation of the Aβ peptide or increases the egress of Aβ peptide will be considered useful as an agent for the treatment of Alzheimer's Disease. To date, there remains a need for the identification of new such agents that are safe and effective to use for the therapeutic intervention of Alzheimer's Disease. The present invention provides novel methods for the treatment of this disorder.

### SUMMARY OF THE INVENTION

The methods of the present invention are directed to reducing Aβ1-40 generation in primary cortico-hippocampal neurons of a mammal comprising administering to said mammal a composition comprising an cardiovascular agent selected from the group consisting of Metergoline; Suloctidil; Bumetanide; Ethacrynic Acid; Tetrandrine; Perhexiline Maleate; Amlodipine Besylate; Bepridil Hydrochloride; Prazosin Hydrochloride; Fendiline Hydrochloride; Candesartan Cilextil; Nicardipine Hydrochloride; Fenofibrate; Amiodarone Hydrochloride; Papaverine Hydrochloride; N,N-Hexamethyleneamiloride; Reserpine; Simvastatin; Cadmium Acetate; Nitrendipine; Propafenone Hydrochloride; Carvedilol; Flunarizine Hydrochloride; Oxidopamine Hydrochloride; Lanatoside C; Lanatoside C; Dicumarol; Valsartan; Propranolol Hydrochloride (−); Veratrine Sulfate; Vinpocetine; Spironolactone; Protoveratrine B; Quinidine Gluconate; Propranolol Hydrochloride (±); Atorvastatin Calcium; Hydralazine Hydrochloride; Yohimbine Hydrochloride; Xylometazoline Hydrochloride; Digitoxin; Nylidrin Hydrochloride; Verapamil; Cyclothiazide; Chrysin; Scopoletin; Dipyridamole; Nifedipine; Althiazide; Losartan; Nicergoline; Bendrofumethiazide; Probucol; Amiloride Hydrochloride; Oxymetazoline Hydrochloride; Isoxsuprine Hydrochloride; Isoxsuprine Hydrochloride; Pargyline Hydrochloride; Nimodipine; Neriifolin; Nicotinyl Tartrate; Isosorbide Dinitrate; Pempidine Tartrate; 2-(2,6-Dimethoxyphenoxyethyl); Aminomethyl-1,4-Benzodioxane; Hydrochloride; Phentolamine Hydrochloride; Disopyramide Phosphate; Rosuvastatin; Perindopril Erbumine; Olmesartan Medoxomil; Hexamethonium Bromide; Labetalol Hydrochloride; Tranexamic Acid; and Dopamine Hydrochloride; analogs thereof and combinations thereof.

Other methods are directed to reducing Aβ1-42 generation in primary cortico-hippocampal neurons of a mammal comprising administering to said mammal a composition comprising an cardiovascular agent selected from the group consisting of Ethacrynic Acid; Metergoline; Cadmium Acetate; Suloctidil; Amlodipine Besylate; Candesartan Cilextil; Bepridil Hydrochloride; Prazosin Hydrochloride; Amiodarone Hydrochloride; Tetrandrine; Perhexiline Maleate; Fendiline Hydrochloride; N,N-Hexamethyleneamiloride; Nicardipine Hydrochloride; Papaverine Hydrochloride; Carvedilol; Propranolol Hydrochloride (−); Oxidopamine Hydrochloride; Reserpine; Valsartan; Oxymetazoline Hydrochloride; Pindolol; Amiloride Hydrochloride; Flunarizine Hydrochloride; Tranexamic Acid; Dicumarol; Propafenone Hydrochloride; Bendrofumethiazide; Dipyridamole; Hydralazine Hydrochloride; Nitrendipine; Triamterene; Althiazide; Rosuvastatin; Disopyramide Phosphate; Isosorbide Dinitrate; Alfluzosin; Yohimbine Hydrochloride; Bucladesine; Quinidine Gluconate; Spironolactone; Olmesartan Medoxomil; Xylometazoline Hydrochloride; Hexamethonium Bromide; Phentolamine Hydrochloride; Nicotinyl Tartrate; Rauwolscine Hydrochloride; Bumetanide; Cyclothiazide; Midodrine Hydrochloride; Atorvastatin Calcium; Fenofibrate; Dopamine Hydrochloride; Pempidine Tartrate; Fenoterol Hydrobromide; Irbesartan; Chrysin; Isoxsuprine Hydrochloride; Isoxsuprine Hydrochloride; and Trichlormethiazide and analogs thereof and combinations thereof.

Also described are methods of treating Alzheimer's disease in a mammal comprising administering to said mammal a composition comprising an cardiovascular agent agent selected from the group consisting of Metergoline; Suloctidil; Bumetanide; Ethacrynic Acid; Tetrandrine; Perhexiline Maleate; Amlodipine Besylate; Bepridil Hydrochloride; Prazosin Hydrochloride; Fendiline Hydrochloride; Candesartan Cilextil; Nicardipine Hydrochloride; Fenofibrate; Amiodarone Hydrochloride; Papaverine Hydrochloride; N,N-Hexamethyleneamiloride; Reserpine; Simvastatin; Cadmium Acetate; Nitrendipine; Propafenone Hydrochloride; Carvedilol; Flunarizine Hydrochloride; Oxidopamine Hydrochloride; Lanatoside C; Lanatoside C; Dicumarol; Valsartan; Propranolol Hydrochloride (−); Veratrine Sulfate; Vinpocetine; Spironolactone; Protoveratrine B; Quinidine Gluconate; Propranolol Hydrochloride (±); Atorvastatin Calcium; Hydralazine Hydrochloride; Yohimbine Hydrochloride; Xylometazoline Hydrochloride; Digitoxin; Nylidrin Hydrochloride; Verapamil; Cyclothiazide; Chrysin; Scopoletin; Dipyridamole; Nifedipine; Althiazide; Losartan; Nicergoline; Bendrofumethiazide; Probucol; Amiloride Hydrochloride; Oxymetazoline Hydrochloride; Isoxsuprine Hydrochloride; Isoxsuprine Hydrochloride; Pargyline Hydrochloride; Nimodipine; Neriifolin; Nicotinyl Tartrate; Isosorbide Dinitrate; Pempidine Tartrate; 2-(2,6-Dimethoxyphenoxyethyl); Aminomethyl-1,4-Benzodioxane; Hydrochloride; Phentolamine Hydrochloride; Disopyramide Phosphate; Rosuvastatin; Perindopril Erbumine; Olmesartan Medoxomil; Hexamethonium Bromide; Labetalol Hydrochloride; Tranexamic Acid; and Dopamine Hydrochloride; analogs thereof and combinations thereof, in an amount effective to ameliorate at least one symptom of said disease in said mammal.

The invention also contemplates method of treating Alzheimer's disease in a mammal comprising administering to said mammal a composition comprising an cardiovascular agent agent selected from the group consisting of Ethacrynic Acid; Metergoline; Cadmium Acetate; Suloctidil; Amlodipine Besylate; Candesartan Cilextil; Bepridil Hydrochloride; Prazosin Hydrochloride; Amiodarone Hydrochloride; Tetrandrine; Perhexiline Maleate; Fendiline Hydrochloride; N,N-Hexamethyleneamiloride; Nicardipine Hydrochloride; Papaverine Hydrochloride; Carvedilol; Propranolol Hydrochloride (−); Oxidopamine Hydrochloride; Reserpine; Valsartan; Oxymetazoline Hydrochloride; Pindolol; Amiloride Hydrochloride; Flunarizine Hydrochloride; Tranexamic Acid; Dicumarol; Propafenone Hydrochloride; Bendrofumethiazide; Dipyridamole; Hydralazine Hydrochloride; Nitrendipine; Triamterene; Althiazide; Rosuvastatin; Disopyramide Phosphate; Isosorbide Dinitrate; Alfluzosin; Yohimbine Hydrochloride; Bucladesine; Quinidine Gluconate; Spironolactone; Olmesartan Medoxomil; Xylometazoline Hydrochloride; Hexamethonium Bromide; Phentolamine Hydrochloride; Nicotinyl Tartrate; Rauwolscine Hydrochloride; Bumetanide; Cyclothiazide; Midodrine Hydrochloride; Atorvastatin Calcium; Fenofibrate; Dopamine Hydrochloride; Pempidine Tartrate; Fenoterol Hydrobromide; Irbesartan; Chrysin; Isoxsuprine Hydrochloride; Isoxsuprine Hydrochloride; and Trichlormethiazide and analogs thereof and combinations thereof.

In specific embodiments, the administration of said cardiovascular agent to said animal decreases Aβ generation in the brain of said mammal to decrease or prevent the likelihood of AD amyloid neuropathy in said mammal.

In other embodiments, the administration of said cardiovascular agent to said animal increase Aβ clearance from the brain, to decrease or prevent the likelihood of AD amyloid neuropathy in said mammal.

In still other embodiments, the administration of said cardiovascular agent to said animal decreases cognitive deterioration in the mammal as compared to the cognitive deterioration of a mammal with AD in the absence of said administration of said cardiovascular agent.

The efficacy of the treatment is determined by the improvement, or reduction or arrest of deterioration in at least one of the assessments selected from the group consisting of the Alzheimer's Disease Assessment Scale-cognitive subscale (ADAS-cog), the Alzheimer's Disease Cooperative Study-Activities of Daily Living (ADCS-ADL) Inventory and Clinician's Interview-Based Impression of Change Plus Version (CIBIC-plus).

In the methods of the present invention, the administration of said cardiovascular agent to said animal preferably increase Aβ clearance from the brain, to decrease or prevent the likelihood of AD amyloid neuropathy in said mammal.

In specific embodiments, the dose of cardiovascular agent may be one that is substantially lower than the dose of the agent typically recommended for use in hypertension. For example, in the methods of the present invention, the dose of the cardiovascular agent used is at least 2-fold less than the dose of said agent recommended used for use in hypertension. In other specific embodiments, the administration said cardiovascular agent reduces the ratio of Aβ1-42 to Aβ1-40 as % value as compared to control mammals that do not receive the cardiovascular agent. Preferably, the ratio of Aβ1-34 and Aβ1-38 to Aβ1-40 remains unaffected. In other methods of the invention, it is seen that the method produces a reduction in the amount of HMW Aβ oligomer formation in the cerebral cortex of said mammal.

Also disclosed is use an cardiovascular agent selected from the group consisting of of Ethacrynic Acid; Metergoline; Cadmium Acetate; Suloctidil; Amlodipine Besylate; Candesartan Cilextil; Bepridil Hydrochloride; Prazosin Hydrochloride; Amiodarone Hydrochloride; Tetrandrine; Perhexiline Maleate; Fendiline Hydrochloride; N,N-Hexamethyleneamiloride; Nicardipine Hydrochloride; Papaverine Hydrochloride; Carvedilol; Propranolol Hydrochloride (−); Oxidopamine Hydrochloride; Reserpine; Valsartan; Oxymetazoline Hydrochloride; Pindolol; Amiloride Hydrochloride; Flunarizine Hydrochloride; Tranexamic Acid; Dicumarol; Propafenone Hydrochloride; Bendrofumethiazide; Dipyridamole; Hydralazine Hydrochloride; Nitrendipine; Triamterene; Althiazide; Rosuvastatin; Disopyramide Phosphate; Isosorbide Dinitrate; Alfluzosin; Yohimbine Hydrochloride; Bucladesine; Quinidine Gluconate; Spironolactone; Olmesartan Medoxomil; Xylometazoline Hydrochloride; Hexamethonium Bromide; Phentolamine Hydrochloride; Nicotinyl Tartrate; Rauwolscine Hydrochloride; Bumetanide; Cyclothiazide; Midodrine Hydrochloride; Atorvastatin Calcium; Fenofibrate; Dopamine Hydrochloride; Pempidine Tartrate; Fenoterol Hydrobromide; Irbesartan; Chrysin; Isoxsuprine Hydrochloride; Isoxsuprine Hydrochloride; and Trichlormethiazide and analogs and combinations thereof for the manufacture of a medicament for the treatment of Alzheimer's Disease.

Also disclosed is use of a cardiovascular agent selected from the group consisting of of Ethacrynic Acid; Metergoline; Cadmium Acetate; Suloctidil; Amlodipine Besylate; Candesartan Cilextil; Bepridil Hydrochloride; Prazosin Hydrochloride; Amiodarone Hydrochloride; Tetrandrine; Perhexiline Maleate; Fendiline Hydrochloride; N,N-Hexamethyleneamiloride; Nicardipine Hydrochloride; Papaverine Hydrochloride; Carvedilol; Propranolol Hydrochloride (−); Oxidopamine Hydrochloride; Reserpine; Valsartan; Oxymetazoline Hydrochloride; Pindolol; Amiloride Hydrochloride; Flunarizine Hydrochloride; Tranexamic Acid; Dicumarol; Propafenone Hydrochloride; Bendrofumethiazide; Dipyridamole; Hydralazine Hydrochloride; Nitrendipine; Triamterene; Althiazide; Rosuvastatin; Disopyramide Phosphate; Isosorbide Dinitrate; Alfluzosin; Yohimbine Hydrochloride; Bucladesine; Quinidine Gluconate; Spironolactone; Olmesartan Medoxomil; Xylometazoline Hydrochloride; Hexamethonium Bromide; Phentolamine Hydrochloride; Nicotinyl Tartrate; Rauwolscine Hydrochloride; Bumetanide; Cyclothiazide; Midodrine Hydrochloride; Atorvastatin Calcium; Fenofibrate; Dopamine Hydrochloride; Pempidine Tartrate; Fenoterol Hydrobromide; Irbesartan; Chrysin; Isoxsuprine Hydrochloride; Isoxsuprine Hydrochloride; and Trichlormethiazide and analogs and combinations for the treatment of Alzheimer's Disease.

Also disclosed is use of a cardiovascular agent selected from the group consisting of Metergoline; Suloctidil; Bumetanide; Ethacrynic Acid; Tetrandrine; Perhexiline Maleate; Amlodipine Besylate; Bepridil Hydrochloride; Prazosin Hydrochloride; Fendiline Hydrochloride; Candesartan Cilextil; Nicardipine Hydrochloride; Fenofibrate; Amiodarone Hydrochloride; Papaverine Hydrochloride; N,N-Hexamethyleneamiloride; Reserpine; Simvastatin; Cadmium Acetate; Nitrendipine; Propafenone Hydrochloride; Carvedilol; Flunarizine Hydrochloride; Oxidopamine Hydrochloride; Lanatoside C; Lanatoside C; Dicumarol; Valsartan; Propranolol Hydrochloride (−); Veratrine Sulfate; Vinpocetine; Spironolactone; Protoveratrine B; Quinidine Gluconate; Propranolol Hydrochloride (±); Atorvastatin Calcium; Hydralazine Hydrochloride; Yohimbine Hydrochloride; Xylometazoline Hydrochloride; Digitoxin; Nylidrin Hydrochloride; Verapamil; Cyclothiazide; Chrysin; Scopoletin; Dipyridamole; Nifedipine; Althiazide; Losartan; Nicergoline; Bendrofumethiazide; Probucol; Amiloride Hydrochloride; Oxymetazoline Hydrochloride; Isoxsuprine Hydrochloride; Isoxsuprine Hydrochloride; Pargyline Hydrochloride; Nimodipine; Neriifolin; Nicotinyl Tartrate; Isosorbide Dinitrate; Pempidine Tartrate; 2-(2,6-Dimethoxyphenoxyethyl); Aminomethyl-1,4-Benzodioxane; Hydrochloride; Phentolamine Hydrochloride; Disopyramide Phosphate; Rosuvastatin; Perindopril Erbumine; Olmesartan Medoxomil; Hexamethonium Bromide; Labetalol Hydrochloride; Tranexamic Acid; and Dopamine Hydrochloride; analogs thereof and combinations thereof for the manufacture of a medicament for the treatment of Alzheimer's Disease.

Another aspect of the invention is use of a cardiovascular agent selected from the group consisting of Metergoline; Suloctidil; Bumetanide; Ethacrynic Acid; Tetrandrine; Perhexiline Maleate; Amlodipine Besylate; Bepridil Hydrochloride; Prazosin Hydrochloride; Fendiline Hydrochloride; Candesartan Cilextil; Nicardipine Hydrochloride; Fenofibrate; Amiodarone Hydrochloride; Papaverine Hydrochloride; N,N-Hexamethyleneamiloride; Reserpine; Simvastatin; Cadmium Acetate; Nitrendipine; Propafenone Hydrochloride; Carvedilol; Flunarizine Hydrochloride; Oxidopamine Hydrochloride; Lanatoside C; Lanatoside C; Dicumarol; Valsartan; Propranolol Hydrochloride (−); Veratrine Sulfate; Vinpocetine; Spironolactone; Protoveratrine B; Quinidine Gluconate; Propranolol Hydrochloride (±); Atorvastatin Calcium; Hydralazine Hydrochloride; Yohimbine Hydrochloride; Xylometazoline Hydrochloride; Digitoxin; Nylidrin Hydrochloride; Verapamil; Cyclothiazide; Chrysin; Scopoletin; Dipyridamole; Nifedipine; Althiazide; Losartan; Nicergoline; Bendrofumethiazide; Probucol; Amiloride Hydrochloride; Oxymetazoline Hydrochloride; Isoxsuprine Hydrochloride; Isoxsuprine Hydrochloride; Pargyline Hydrochloride; Nimodipine; Neriifolin; Nicotinyl Tartrate; Isosorbide Dinitrate; Pempidine Tartrate; 2-(2,6-Dimethoxyphenoxyethyl); Aminomethyl-1,4-Benzodioxane; Hydrochloride; Phentolamine Hydrochloride; Disopyramide Phosphate; Rosuvastatin; Perindopril Erbumine; Olmesartan Medoxomil; Hexamethonium Bromide; Labetalol Hydrochloride; Tranexamic Acid; and Dopamine Hydrochloride; analogs thereof and combinations thereof for the treatment of Alzheimer's Disease.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

The following drawings form part of the present specification and are included to further illustrate aspects of the present invention. The invention may be better understood by reference to the drawings in combination with the detailed description of the specific embodiments presented herein.

FIG. 1 Tg2576 mice are characterized by normal blood pressure under basal conditions. Baseline measurements of systolic, diastolic blood pressure, and mean arteriole blood pressure (MAP) in ˜11 month old female Tg2576 mice. Strain-, age-, gender-matched WT mice were measured using a commercial blood pressure analysis system designed specifically for small rodents (Hatteras Instruments, NC). Following manufacturer's instruction, mice were temporarily immobilized in a restraining chamber and an inflated tail-cuff wrapped around the tail was used to measure the systolic pressure, diastolic pressure, and MAP. Each blood pressure determination was calculated as the mean of 10 individual measurements per animal (n=6-10 group).

FIG. 2 Chronic valsartan treatment is highly tolerable in Tg 2576 mice. In this study ˜7-month old female Tg2576 mice were provided with 10 or 40 mg valsartan/kg/day by incorporation of valsartan into the drinking water (50 and 200 mg/L, respectively) prior to the development of AD-type amyloid neuropathology and cognitive decline. Treatment continued for 4 months until ˜11-months of age. Both food and drinking water (±valsartan) were available ad libitum throughout the entire treatment period; A) water consumption B) Body weights throughout the entire 4-month treatment period. Bar graphs represent mean±SEM values, n=6-10 mice per group.

FIG. 3 Valsartan treatment attenuates AD-type spatial memory deterioration and Aβ-neuropathology in a dose dependent fashion in Tg2576 mice. In this study the behavioral and neuropathological impact of valsartan treatment (0, 10 and 40 mg/kg-day) was assessed in ˜11-month old female Tg2576 mice after 4 months of treatment. Cognitive behavioral function was assessed using the Morris water maze (MWM). After completion of the MWM test, blood pressure measurements were quantified as describe in FIG. 1 legends. Following MWM testing and blood pressure assessment, mice were sacrificed for neuropathological assessment. Aβ1-42 and Aβ1-40 ELISA and APP western blot analysis were conducted as previously described (Wang et al., 2005; Appendix 3). A) assessments of spatial memory behavioral performance by MWM paradigm (Ho et al., 2004) in ˜11-month old control Tg2576 mice (untreated) and in Tg2576 mice which underwent treatment with 10- or 40-mg/kg-day valsartan salt in the drinking water for ˜4 months. B) Measurements of blood pressure in response to ˜4 months of valsartan treatment C-D) Aβ1-42 and Aβ1-40 peptide content in the brain assessed using commercial ELISA assays as previously described (Wang et al., 2005; Appendix 3) in the brain of ˜11-month old Tg2576 mice in response to valsartan treatments; total amyloid precursor protein (C8)-immunoreactive (APP) was assessed by western blot analysis while β-actin immunoreactivity served as an internal control. C, (inset), total immunoreactive APP content normalized to β-actin signal. In C-D, bar graphs represent group mean±SEM, n=6-10 mice per group.

FIG. 4. Comparable microvasculature abnormalities in the brain of Tg2576 mice and AD brain. Top panel; total length of capillaries (m) in the CA1 field of 6 human cases (CDR 0, cognitive normal control cases, n=3; CDR 0.5 (MCI), n=3, early AD cases), and four ˜11 month old mice, including n=2 WT and n=2 2 Tg2576 mice. Note the both CDR 0.5 cases and Tg2576 mice tend to show lower capillary lengths. Bottom panel, representative staining of brain silver impregnated vessels in a ˜11 months old Tg2576 mouse illustrating several collapsed (arrows) and fragmented vessels. Scale bar=30 μm.

FIG. 5. Valsartan reduces generation of Ab peptides, possibly through mechanisms involving inhibition of β-secretase processing of amyloid precursor protein. Primary Tg2576 neuron cultures were treated with valsartan (100 mM) for 16 hours. A-C) Assessments of cellular α-, β- and γ-secretase activities in response to valsartan treatment. Cellular β-secretase (A), α-secretase (B) and γ-secretase (C) activities in the presence and absence of valsartan treatment were measured as detailed in Wang et al (2005) using commercial assay kits (Biosource). In A-C), data are expressed as % of non-treated control primary cultures; bar graphs represent mean±SEM (n=3 per group). D) Changes in Aβ peptide profiles in response to valsartan (bottom panel) compared to vehicle control treatment (top panel). The profile of Aβ peptides in the condition media was assessed by first immunoprecipitating Aβ peptides using the 4G8 antibodies, followed by mass spectrometry detection of Aβ peptides as described in Wang et al. (2005). The immunoprecipitation-mass spectrometry (IP-MS) spectra were normalized to the internal standard Aβ12-28. MS peaks corresponding to Aβ peptides are indicated with the Aβ peptide sequence number. Peaks labeled as 1-402+ and Insulin2+ represent doubly protonated Aβ1-40 peptide and doubly protonated insulin molecular ions, respectively; Aβ12-28 was added during the IP procedure and used as internal standard (int. std.) ions as previously reported (Wang et al., 2005).

FIG. 6A-6F. Assessment of total body weight as an index of drug tolerability and assessment of systolic, diastolic, and MAP blood pressure following short-term dosing treatments with propranolol-HCL, nicardipine-HCL, or losartan in Tg2576 mice.

FIG. 7. Short-term dosing treatment studies with propranolol-HCL, nicardipine-HCL or losartan treatments significantly reduce Aβ content in the brain and plasma. Changes in Aβ1-42 content in the hippocampal formation (FIG. 7a-c), or cortex (FIG. 7d-f) in response to treatments with propranolol (FIG. 7a, FIG. 7d), nicardipine (FIG. 7b, FIG. 7e), or losartan (FIG. 7c, FIG. 7f). Aβ1-42 peptide content in peripheral blood (plasma) of Tg2576 mice treated with propranolol (FIG. 7g), nicardipine (FIG. 7h), or losartan (FIG. 7i). In a-i, Aβ1-42 peptide content in the brain and plasma was assessed using a commercial ELISA assay, as previously described (Wang et al., 2005). Bar graphs represent group mean±SEM, n=3 mice per group. 2-tailed t-test: *P<0.05.

FIG. 8. Propranolol-HCL detection in brain and plasma following short-term dosing in Tg2576.

FIG. 9 Propranolol-HCL may decrease Aβ content in the brain in part through inhibition of β-secretase activity in the brain. In A, Aβ peptide contents in the cerebral cortex of control (top chromatogram) vs. propranolol-HCL treated, at 10 mg/kg/day and 60 mg/kg/day (middle and bottom chromatograms respectively) in Tg2576 mice were analyzed by MS-IP following immunoprecipitation with 4G8 antibody (Wang et al., 2005). In A, Aβ peptides were normalized to Aβ12-28 peptide, which was added as an internal standard and in B, quantification each respective Aβ peptide species from the IP/MS peptide profile. In C, fluorimetrical assessment of α- β- γ-secretase activities in the neocortical sample of same Tg 2576 mice in response to propranolol-HCL relative to controls. In D, ratio of Aβ relative to Aβ1-40, in the cortical sample. Bar graphs represent group mean±SEM, n=3 mice per group. One Way Anova followed by 2-tailed t-test: *P<0.05, ** P<0.001.

FIG. 10. Long-term treatments with valsartan in Tg2576 mice, at doses below or within those prescribed for hypertension, attenuates AD-type spatial memory deterioration coincidental with significant reductions in HMW-soluble extracellular Aβ species in the brain.

FIG. 11. Valsartan prevents Aβ1-42 peptide into HMW oligomerization, in vitro. (FIG. 11A) Western analysis of Aβ1-42 oligomers in the presence of losartan, valsartan carvedilol, hydralazine, propranolol, nicardipine or amiloride. Bands at 3.5 kDa represent the monomeric Aβ form, whereas the smear between 55 and 130 kDa represents the oligomeric form of Aβ. (FIG. 11B) Valsartan decreases the accumulation of high-molecular-weight Aβ 1-42 species. (B-inset). Representative Western immunoblot of Aβ1-42 in the absence or presence of 10 μM valsartan (lane 2 and 3 respectively; lane 1 presents non-aggregated, no-incubation Aβ as a negative control, see Methods). (FIG. 11C) Quantitative dot blot analysis of valsartan inhibition of Aβ1-42 oligomerization. The same samples used in FIG. 11A were subjected to dot blot analysis using oligomer-specific antibody A11. (FIG. 11C-inset) Representative dot blot image. Results are expressed as % of control (negative control presents non-aggregated, no incubation Aβ) and values represent mean (±SEM).

FIG. 12. Chronic valsartan treatment is highly tolerable in Tg2576 mice. Valsartan was provided to female Tg2576 from 7 to 11.5 months of age at 10 mg/kg/day or 40 mg/kg/day. (FIG. 12A-B) Body weight and fluid consumption were monitored weekly. (FIG. 12C) Post-prandial glucose tolerance response was examined after 5 months valsartan treatment. (FIG. 12D) Tg2576 blood pressure measurements in response to ˜5 months of valsartan treatments. (FIG. 12E) Baseline measurements of systolic, diastolic blood pressure, and mean arterial blood pressure (MAP) in adult female Tg2576 mice and strain-age-gender-matched WT mice. The blood pressure determination for each animal was calculated as the mean of 10 individual measurements. Values represents group mean values (±SEM); n=7-9 mice per group.

FIG. 13. Chronic valsartan treatment of Tg2576 mice resulted in dose-dependent attenuations of AD-type spatial memory deterioration in Tg2576 mice, which is coincidental with significant reductions in HMW-soluble Aβ species and AD-type neuropathology in the brains of Tg2576 mice. (FIG. 13A) The influence of Aβ related spatial memory in response to valsartan treatment at 10 and 40 mg/kg/day vs. the untreated control Tg2576 mice was assessed using Morris water maze test in ˜11-month old female Tg2576 mice. Latency score represents time taken to escape to the platform from the water. (FIG. 13B) Assessments of soluble, extracellular HMW-Aβ peptide contents in the brain using an antibody specific for HMW oligomeric Aβ peptides in a dot blot analysis. (FIG. 13B-inset) Representative dot-blot analysis of HMW-soluble Aβ contents. (FIG. 13C) Assessment of total PBS soluble Aβ peptide using ELISA assay. (FIG. 13D) Assessment of Aβ1-42 and Aβ₁₋₄₀ peptide concentrations in the cerebral cortex and hippocampus of valsartan (10 or 40 mg/kg/day) or control mice. (FIG. 13E) Stereological assessment of cerebral cortex and hippocampal Aβ-amyloid plaque burden in valsartan or control mice expressed as thioflavin-S positive volume as a percentage of regional volume. (FIG. 13E-inset) Representative photograph of thioflavin-S positive Aβ amyloid plaque neuropathology in neocortex (CTX) and hippocampal formation (FIG. 13H) in untreated control (left panel) and valsartan-treated (40 mg/kg/day) Tg2576 mice (right panel). Values represent group mean±SEM, n=7-9 mice per group. In (FIG. 13B) and (FIG. 13C), *p<0.001. In (FIG. 13D) and (FIG. 13E), *P<0.05, **P<0.01. One way ANOVA followed by Newman-Keuls post-hoc analysis.

FIG. 14. Valsartan treatments prevented cognitive impairment and attenuate AD-type neuropathology in part, by promoting membrane-bound insulin degradation enzyme activity. (FIG. 14A) APP contents in the cortex of valsartan treated or untreated control Tg2576 mice. (FIG. 14A-inset), representative immunoreactive APP (C8 antibody) and β-actin signals. (FIG. 14B) Assessments of cellular α-, β-, and γ-secretase activities in the cerebral cortex of Tg2576 mice in response to valsartan treatment. (FIG. 14C) Assessment of Aβ1-42 and Aβ1-40 peptide contents in peripheral blood (serum). (FIG. 14D) Assessments of cell membrane (CM)-associated (left panel) and cytosolic (right panel) IDE activity in the cerebral cortex of Tg2576 mice in response to valsartan treatment. (FIG. 14D-inset) Representative immuoblot signals of membrane and cytosolic IDE protein content from the same samples. (FIG. 14E) Assessments of neprilysin content by western blot using a commercial rabbit anti-mouse NEP antibody. (E-inset) Representative neprilysin and actin protein signals from the same blot. (F) Assessment of endothelin-converting enzyme activity using Endothelin-1 ELISA System. Values represent group mean±SEM, n=7-9 mice per group. *P<0.05, One way ANOVA; followed by Newman-Keuls post-hoc analysis.

### DETAILED DESCRIPTION OF THE PREFERRED EMBODIMENTS OF THE INVENTION

Aβ neuropathology is a major hallmark in the Alzheimer's Disease brain (reviewed in Cumming, 2004; Selkoe, 2001), any agents that can lower the rate of accumulation of this peptide in the brain by decreasing the rates of formation and/or aggregation and/or increasing the rates of egress of the peptide from the brain will be useful as therapeutic agents in the treatment of Alzheimer's Disease. In the present invention, it was discovered that certain specific cardiovascular agents have this therapeutic potential, whereas others do not. These agents were found to have a potential Aβ-lowering activity. As discussed in more detail in the examples presented herein below, the cardiovascular agents tested represent a wide spectrum of pharmacological profiles, one of which is antihypertensive activity. It was observed that cardiovascular agents are capable of significantly reducing Aβ1-40 and/or Aβ1-42 generation (by >15%) in primary cortico-hippocampal neuron cultures generated from mouse models of Alzhiemer's Disease. This exciting discovery has far-reaching potential in the treatment of Alzheimer's Disease, not least because these agents are well-characterized agents that are already commercially available as therapeutic agents used in other indications.

In exemplary embodiments, cardiovascular agents were examined for their for Aβ-lowering activity. The effective concentrations of agents resulting in a 50% inhibition (EC50) of Aβ1-40 and for Aβ1-42 content in the conditioning medium of the neuron cultures were calculated, relative to parallel vehicle-treated transgenic Alzheimer's disease control cultures. Numerous “cardiovascular drugs” exerted dose-dependent Aβ1-40 and/or Aβ1-42 lowering activity with a predicted EC50 at <10 μM (Table 1). No apparent neurotoxicity was associated with any of the agents, as assessed by a lactate dehydrogenase (LDH) activity assay in parallel cultures at identical drug concentrations (Table 1). This evidence is of great interest and supports the use of cardiovascular agents to influence Aβ generation/clearance from the brain, ultimately resulting in an amelioration, treatment or even prevention of Alzheimer's Disease amyloid neuropathology. The present invention is directed to methods and compositions that use this finding to provide novel therapeutic methods for the treatment of Alzheimer's Disease.

The present invention thus is directed to the use of cardiovascular agents for the amelioration, treatment, prevention or other therapeutic intervention of Alzheimer's Disease. Several classes of compounds are known to have activity as cardiovascular agents. These include calcium channel blockers, ACE inhibitors, A-II antagonists, diuretics, beta-adrenergic receptor blockers, vasodilators and alpha-adrenergic receptor blockers, statins and the like. There are many commercially-available examples of agents in each of these classes of cardiovascular agents.

However, in the present invention it is shown that there are only certain agents from within each of these classes of agents that have a therapeutic anti-AD effect. The following table shows the effects of cardiovascular drugs that affect Aβ1-42 production.

Table showing effects of cardiovascular drugs that reduce Aβ1-42 (<85%)

Table showing effects of cardiovascular
drugs that reduce Aβ1-42 (<85%)

| | Ab1-40 | Ab1-42 | LDH | MTT |
|---|---|---|---|---|
| | (% of | (% of | (% of | (% of |
| Drug Name | ctl) | ctl) | ctl) | ctl) |
| Cardiovascular Drugs That | | | | |
| Reduce Aβ1-42 (<85%) | | | | |
| Ethacrynic Acid | 4.4 | 1.8 | 101.5 | 109.9 |
| Metergoline | −3.1 | 2.3 | 159.3 | 76.5 |
| Cadmium Acetate | 29.5 | 3.8 | 889.8 | 29.1 |
| Suloctidil | 1.8 | 4.6 | 396.1 | 77.7 |
| Amlodipine Besylate | 4.9 | 10.2 | 1188.9 | 13.3 |
| Candesartan Cilextil | 9.3 | 18.1 | 227.6 | 41.2 |
| Bepridil Hydrochloride | 5.1 | 19.9 | 1885.2 | 77.1 |
| Prazosin Hydrochloride | 7.6 | 23.2 | 260.2 | 99.9 |
| Amiodarone Hydrochloride | 14.9 | 23.2 | | |
| Tetrandrine | 4.5 | 25.2 | 117.6 | 112.5 |
| Perhexiline Maleate | 4.8 | 26.2 | 1065.9 | 7.2 |
| Fendiline Hydrochloride | 7.9 | 26.2 | 1881.4 | 7.7 |
| N,N-Hexamethyleneamiloride | 15.4 | 26.2 | 117.3 | 96.1 |
| Nicardipine Hydrochloride | 10.7 | 27.6 | 104.2 | 52.4 |
| Papaverine Hydrochloride | 15.3 | 30.1 | 118.4 | 92.2 |
| Carvedilol | 38.3 | 30.8 | 148.6 | 66.7 |
| Propranolol Hydrochloride (−) | 55.0 | 32.8 | 166.6 | 77.4 |
| Oxidopamine Hydrochloride | 43.8 | 36.4 | 103.6 | 121.5 |
| Reserpine | 24.7 | 38.7 | 157.8 | 87.2 |
| Valsartan | 51.6 | 42.0 | 94.1 | 67.4 |
| Oxymetazoline Hydrochloride | 78.0 | 44.9 | 97.0 | 97.3 |
| Pindolol | 99.0 | 46.1 | 171.7 | 98.9 |
| Amiloride Hydrochloride | 77.5 | 47.1 | 104.7 | 80.8 |
| Flunarizine Hydrochloride | 42.1 | 53.3 | 120.4 | 59.4 |
| Tranexamic Acid | 84.9 | 53.9 | 112.8 | 96.8 |
| Dicumarol | 51.3 | 54.0 | 93.3 | 97.0 |
| Propafenone Hydrochloride | 36.3 | 56.4 | 113.7 | 95.2 |
| Bendrofumethiazide | 76.2 | 58.4 | 104.7 | 88.9 |
| Dipyridamole | 72.2 | 62.6 | 107.5 | 102.7 |
| Hydralazine Hydrochloride | 66.1 | 63.2 | 101.1 | 95.7 |
| Nitrendipine | 33.0 | 64.0 | 94.3 | 100.8 |
| Triamterene | 85.1 | 64.2 | 103.3 | 93.8 |
| Althiazide | 74.4 | 65.4 | 109.0 | 99.7 |
| Rosuvastatin | 83.6 | 65.6 | 298.7 | 103.8 |
| Disopyramide Phosphate | 83.5 | 66.3 | 125.8 | 71.2 |
| Isosorbide Dinitrate | 80.5 | 66.8 | 124.6 | 100.8 |
| Alfluzosin | 122.7 | 66.9 | 95.4 | 91.8 |
| Yohimbine Hydrochloride | 66.2 | 68.9 | 107.8 | 82.7 |
| Bucladesine | 85.3 | 69.9 | 357.1 | 97.2 |
| Quinidine Gluconate | 63.3 | 70.0 | 357.6 | 46.8 |
| Spironolactone | 60.4 | 70.7 | 100.6 | 117.0 |
| Olmesartan Medoxomil | 83.9 | 70.7 | 90.7 | 90.7 |
| Xylometazoline Hydrochloride | 66.6 | 71.8 | 110.8 | 88.2 |
| Hexamethonium Bromide | 84.8 | 72.6 | 99.7 | 92.1 |
| Phentolamine Hydrochloride | 83.4 | 72.9 | 90.7 | 110.3 |
| Nicotinyl Tartrate | 80.0 | 73.8 | 122.6 | 92.7 |
| Rauwolscine Hydrochloride | 103.2 | 74.3 | 109.4 | 98.0 |
| Bumetanide | 2.3 | 74.7 | 118.1 | 95.3 |
| Cyclothiazide | 71.1 | 75.0 | 110.3 | 97.7 |
| Midodrine Hydrochloride | 86.0 | 76.7 | 105.6 | 87.8 |
| Atorvastatin Calcium | 66.0 | 76.9 | 99.4 | 98.4 |
| Fenofibrate | 13.9 | 77.7 | 140.0 | 97.5 |
| Dopamine Hydrochloride | 85.0 | 78.3 | 559.5 | 95.7 |
| Pempidine Tartrate | 80.9 | 78.6 | 108.4 | 91.3 |
| Fenoterol Hydrobromide | 89.3 | 78.8 | 104.3 | 93.9 |
| Irbesartan | 89.7 | 82.3 | 112.5 | 98.9 |
| Chrysin | 71.6 | 83.4 | 118.3 | 84.5 |
| Isoxsuprine Hydrochloride | 78.3 | 83.7 | 106.2 | 99.8 |
| Isoxsuprine Hydrochloride | 78.3 | 83.7 | 107.7 | 93.1 |
| Trichlormethiazide | 89.3 | 84.5 | 102.6 | 105.5 |
| Cardiovascular Drugs That Has | | | | |
| No Effect On Aβ1-42 (85-105%) | | | | |
| Phenylbutyrate Sodium | 102.1 | 86.6 | 192.3 | 70.2 |
| Veratrine Sulfate | 58.5 | 87.1 | 124.4 | 84.4 |
| Strophanthidin | 107.5 | 88.6 | 119.4 | 99.2 |
| Perindopril Erbumine | 83.8 | 88.8 | 72.5 | 95.3 |
| Hydroflumethiazide | 92.4 | 90.2 | 99.7 | 98.9 |
| Neriifolin | 79.7 | 90.3 | 110.8 | 101.2 |
| Nylidrin Hydrochloride | 70.5 | 90.4 | 115.6 | 79.7 |
| Nifedipine | 73.4 | 90.6 | 76.1 | 98.7 |
| Phenoxybenzamine Hydrochloride | 87.5 | 90.7 | 81.3 | 72.8 |
| Nimodipine | 79.1 | 90.9 | 100.4 | 115.6 |
| Peruvoside | 115.3 | 90.9 | 105.5 | 98.8 |
| Indapamide | 107.0 | 91.3 | 85.3 | 104.2 |
| Tamsulosin Hydrchloride | 106.5 | 91.8 | 102.2 | 96.1 |
| Clofibrate | 90.9 | 92.5 | 110.5 | 97.9 |
| Digitoxin | 68.3 | 92.9 | 106.4 | 86.0 |
| Sulmazole | 115.6 | 93.2 | 111.1 | 115.0 |
| Protoveratrine B | 61.8 | 93.3 | 729.8 | 58.5 |
| Propranolol Hydrochloride (+/−) | 65.2 | 93.4 | 98.1 | 105.9 |
| Ellagic Acid | 96.6 | 94.0 | 109.2 | 85.2 |
| Atenolol | 90.4 | 94.5 | 136.8 | 97.3 |
| Pronetalol Hydrochloride | 88.0 | 94.6 | 108.9 | 88.8 |
| 2-(2,6-Dimethoxyphenoxyethyl) | 82.6 | 94.7 | 103.9 | 95.5 |
| Aminomethyl-1,4-Benzodioxane | | | | |
| Hydrochloride | | | | |
| Niacin | 101.1 | 95.0 | 134.5 | 61.0 |
| Fosinopril Sodium | 94.7 | 95.0 | 75.6 | 101.4 |
| Furosemide | 118.5 | 95.7 | 98.2 | 101.0 |
| Fenbutyramide | 91.6 | 96.3 | 135.0 | 108.6 |
| Chlorthalidone | 108.5 | 96.7 | 645.8 | 12.1 |
| Losartan | 76.0 | 97.1 | 110.4 | 71.3 |
| Enalapril Maleate | 100.7 | 97.6 | 118.9 | 126.4 |
| Metolazone | 104.0 | 98.0 | 103.7 | 69.7 |
| Nicergoline | 76.1 | 98.5 | 106.2 | 99.8 |
| Tulobuterol | 95.5 | 98.6 | 95.3 | 88.4 |
| Betahistine Hydrochloride | 110.4 | 98.9 | 107.3 | 107.1 |
| Clopamide | 92.0 | 98.9 | 99.9 | 99.2 |
| Minoxidil | 94.2 | 98.9 | 117.1 | 98.5 |
| Benzthiazide | 105.7 | 98.9 | 105.1 | 98.5 |
| Diltiazem Hydrochloride | 107.8 | 98.9 | 96.1 | 97.8 |
| Diazoxide | 113.5 | 98.9 | 112.0 | 97.8 |
| BENZAFIBRATE | 107.2 | 99.5 | | |
| Tolazoline Hydrochloride | 90.8 | 99.6 | 112.5 | 97.5 |
| Captopril | 104.5 | 99.7 | 120.2 | 62.3 |
| Practolol | 99.7 | 99.9 | 108.9 | 101.3 |
| Lanatoside C | 45.8 | 100.3 | 166.0 | 66.5 |
| Lanatoside C | 45.8 | 100.3 | 166.0 | 66.5 |
| Verapamil | 70.9 | 101.1 | 92.5 | 84.3 |
| Anisindione | 90.1 | 103.2 | 148.9 | 110.7 |
| Ajmaline | 114.0 | 103.3 | 97.6 | 90.3 |
| Dobutamine Hydrochloride | 90.1 | 103.3 | 103.1 | 99.5 |
| Timolol Maleate | 101.3 | 103.8 | 104.4 | 99.2 |
| Torsemide | 107.4 | 103.9 | 107.5 | 99.2 |
| Vincamine | 99.1 | 104.3 | 96.4 | 98.5 |
| Chlorothiazide | 107.3 | 104.3 | 110.3 | 99.4 |
| Metoprolol Tartrate | 91.4 | 104.4 | 107.5 | 94.7 |
| 2-HYDROXY-4-(2-HYDROXY-3-T- | 99.2 | 104.4 | 109.3 | 97.1 |
| BUTYLAMINOPROPOXY)- | | | | |
| BENZIMIDAZOLE | | | | |
| Protoveratrine A | 91.7 | 104.6 | | |
| Phenindione | 104.0 | 104.7 | 107.9 | 108.8 |
| Vinpocetine | 60.4 | 104.8 | 111.3 | 87.7 |
| Urea | 92.8 | 104.9 | 130.8 | 96.7 |
| Ramipril | 107.4 | 105.0 | 96.4 | 102.9 |
| Cardiovascular Drugs That | | | | |
| Promote Aβ1-42 (>105%) | | | | |
| Heptaminol Hydrochloride | 112.2 | 105.4 | 103.4 | 103.4 |
| Pargyline Hydrochloride | 78.6 | 105.5 | 116.9 | 86.4 |
| Labetalol Hydrochloride | 84.9 | 105.5 | 164.0 | 93.0 |
| Alprenolol | 93.8 | 105.6 | 128.0 | 92.2 |
| Canrenoic Acid, Potassium Salt | 100.6 | 106.5 | 106.9 | 102.1 |
| Warfarin | 89.4 | 107.0 | 106.6 | 100.1 |
| Procainamide Hydrochloride | 118.1 | 108.6 | 111.1 | 96.8 |
| Pentoxifylline | 97.1 | 109.0 | 132.4 | 91.1 |
| Scopoletin | 71.9 | 109.4 | 106.2 | 99.7 |
| Todralazine Hydrochloride | 104.9 | 109.8 | 100.6 | 99.5 |
| Trandolapril | 136.7 | 110.6 | 95.5 | 91.5 |
| Methyldopa | 109.2 | 111.0 | 118.9 | 88.5 |
| Telmisartan | 91.1 | 112.0 | 111.3 | 109.4 |
| Nadolol | 94.2 | 113.2 | 106.5 | 102.8 |
| Urapidil | 96.4 | 113.9 | 97.1 | 103.4 |
| Benazepril Hydrochloride | 116.3 | 114.9 | 103.5 | 99.5 |
| Mexiletine Hydrochloride | 91.3 | 115.3 | 103.4 | 95.6 |
| Hydrochlorothiazide | 89.5 | 115.6 | 281.1 | 102.1 |
| Theobromine | 101.3 | 116.7 | 118.3 | 97.2 |
| Acetazolamide | 114.7 | 118.0 | 173.5 | 78.0 |
| Quinapril Hydrochloride | 119.3 | 118.0 | 101.8 | 102.4 |
| Molsidomine | 118.3 | 120.6 | 1127.4 | 15.0 |
| Hydroquinidine | 96.5 | 120.7 | 102.2 | 103.6 |
| Tulobuterol | 101.5 | 121.7 | 384.1 | 37.0 |
| Simvastatin | 28.0 | 122.0 | 129.9 | 93.4 |
| 1S,9R-Beta-HYDRASTINE | 114.5 | 123.6 | 114.2 | 101.1 |
| Mecamylamine Hydrochloride | 124.4 | 129.6 | 97.0 | 114.9 |
| Pentolinium Tartrate | 121.0 | 133.9 | 118.0 | 103.8 |
| Probucol | 76.8 | 135.8 | 109.3 | 94.8 |
| Deltaline | 121.0 | 143.0 | 118.9 | 99.4 |
| Nafronyl Oxalate | 133.5 | 144.6 | 96.6 | 103.2 |
| Guanethidine Sulfate | 125.5 | 149.5 | 1057.3 | 12.9 |
| Berbamine Hydrochloride | 119.1 | 150.6 | 129.1 | 97.6 |
| Acebutolol Hydrochloride | 133.6 | 165.2 | 107.6 | 99.4 |
| Aminocaproic Acid | 124.4 | 184.3 | 90.4 | 80.3 |

Thus, agents that were useful in reducing Aβ1-42 were Ethacrynic Acid; Metergoline; Cadmium Acetate; Suloctidil; Amlodipine Besylate; Candesartan Cilextil; Bepridil Hydrochloride; Prazosin Hydrochloride; Amiodarone Hydrochloride; Tetrandrine; Perhexiline Maleate; Fendiline Hydrochloride; N,N-Hexamethyleneamiloride; Nicardipine Hydrochloride; Papaverine Hydrochloride; Carvedilol; Propranolol Hydrochloride (−); Oxidopamine Hydrochloride; Reserpine; Valsartan; Oxymetazoline Hydrochloride; Pindolol; Amiloride Hydrochloride; Flunarizine Hydrochloride; Tranexamic Acid; Dicumarol; Propafenone Hydrochloride; Bendrofumethiazide; Dipyridamole; Hydralazine Hydrochloride; Nitrendipine; Triamterene; Althiazide; Rosuvastatin; Disopyramide Phosphate; Isosorbide Dinitrate; Alfluzosin; Yohimbine Hydrochloride; Bucladesine; Quinidine Gluconate; Spironolactone; Olmesartan Medoxomil; Xylometazoline Hydrochloride; Hexamethonium Bromide; Phentolamine Hydrochloride; Nicotinyl Tartrate; Rauwolscine Hydrochloride; Bumetanide; Cyclothiazide; Midodrine Hydrochloride; Atorvastatin Calcium; Fenofibrate; Dopamine Hydrochloride; Pempidine Tartrate; Fenoterol Hydrobromide; Irbesartan; Chrysin; Isoxsuprine Hydrochloride; Isoxsuprine Hydrochloride; and Trichlormethiazide.

The following table shows the effects of cardiovascular drugs that affect Aβ1-40 production.

Table of cardiovascular drugs that affect Aβ1-40 production.
Primary Screening

| | Ab1-40 | Ab1-42 | LDH | MTT |
|---|---|---|---|---|
| | (% of | (% of | (% of | (% of |
| Drug Name | ctl) | ctl) | ctl) | ctl) |
| Cardiovascular Drugs That | | | | |
| Reduce Aβ1-40(<85%) | | | | |
| Metergoline | −3.1 | 2.3 | 159.3 | 76.5 |
| Suloctidil | 1.8 | 4.6 | 396.1 | 77.7 |
| Bumetanide | 2.3 | 74.7 | 118.1 | 95.3 |
| Ethacrynic Acid | 4.4 | 1.8 | 101.5 | 109.9 |
| Tetrandrine | 4.5 | 25.2 | 117.6 | 112.5 |
| Perhexiline Maleate | 4.8 | 26.2 | 1065.9 | 7.2 |
| Amlodipine Besylate | 4.9 | 10.2 | 1188.9 | 13.3 |
| Bepridil Hydrochloride | 5.1 | 19.9 | 1885.2 | 77.1 |
| Prazosin Hydrochloride | 7.6 | 23.2 | 260.2 | 99.9 |
| Fendiline Hydrochloride | 7.9 | 26.2 | 1881.4 | 7.7 |
| Candesartan Cilextil | 9.3 | 18.1 | 227.6 | 41.2 |
| Nicardipine Hydrochloride | 10.7 | 27.6 | 104.2 | 52.4 |
| Fenofibrate | 13.9 | 77.7 | 140.0 | 97.5 |
| Amiodarone Hydrochloride | 14.9 | 23.2 | | |
| Papaverine Hydrochloride | 15.3 | 30.1 | 118.4 | 92.2 |
| N,N-Hexamethyleneamiloride | 15.4 | 26.2 | 117.3 | 96.1 |
| Reserpine | 24.7 | 38.7 | 157.8 | 87.2 |
| Simvastatin | 28.0 | 122.0 | 129.9 | 93.4 |
| Cadmium Acetate | 29.5 | 3.8 | 889.8 | 29.1 |
| Nitrendipine | 33.0 | 64.0 | 94.3 | 100.8 |
| Propafenone Hydrochloride | 36.3 | 56.4 | 113.7 | 95.2 |
| Carvedilol | 38.3 | 30.8 | 148.6 | 66.7 |
| Flunarizine Hydrochloride | 42.1 | 53.3 | 120.4 | 59.4 |
| Oxidopamine Hydrochloride | 43.8 | 36.4 | 103.6 | 121.5 |
| Lanatoside C | 45.8 | 100.3 | 166.0 | 66.5 |
| Lanatoside C | 45.8 | 100.3 | 166.0 | 66.5 |
| Dicumarol | 51.3 | 54.0 | 93.3 | 97.0 |
| Valsartan | 51.6 | 42.0 | 94.1 | 67.4 |
| Propranolol Hydrochloride (−) | 55.0 | 32.8 | 166.6 | 77.4 |
| Veratrine Sulfate | 58.5 | 87.1 | 124.4 | 84.4 |
| Vinpocetine | 60.4 | 104.8 | 111.3 | 87.7 |
| Spironolactone | 60.4 | 70.7 | 100.6 | 117.0 |
| Protoveratrine B | 61.8 | 93.3 | 729.8 | 58.5 |
| Quinidine Gluconate | 63.3 | 70.0 | 357.6 | 46.8 |
| Propranolol Hydrochloride (+/−) | 65.2 | 93.4 | 98.1 | 105.9 |
| Atorvastatin Calcium | 66.0 | 76.9 | 99.4 | 98.4 |
| Hydralazine Hydrochloride | 66.1 | 63.2 | 101.1 | 95.7 |
| Yohimbine Hydrochloride | 66.2 | 68.9 | 107.8 | 82.7 |
| Xylometazoline Hydrochloride | 66.6 | 71.8 | 110.8 | 88.2 |
| Digitoxin | 68.3 | 92.9 | 106.4 | 86.0 |
| Nylidrin Hydrochloride | 70.5 | 90.4 | 115.6 | 79.7 |
| Verapamil | 70.9 | 101.1 | 92.5 | 84.3 |
| Cyclothiazide | 71.1 | 75.0 | 110.3 | 97.7 |
| Chrysin | 71.6 | 83.4 | 118.3 | 84.5 |
| Scopoletin | 71.9 | 109.4 | 106.2 | 99.7 |
| Dipyridamole | 72.2 | 62.6 | 107.5 | 102.7 |
| Nifedipine | 73.4 | 90.6 | 76.1 | 98.7 |
| Althiazide | 74.4 | 65.4 | 109.0 | 99.7 |
| Losartan | 76.0 | 97.1 | 110.4 | 71.3 |
| Nicergoline | 76.1 | 98.5 | 106.2 | 99.8 |
| Bendrofumethiazide | 76.2 | 58.4 | 104.7 | 88.9 |
| Probucol | 76.8 | 135.8 | 109.3 | 94.8 |
| Amiloride Hydrochloride | 77.5 | 47.1 | 104.7 | 80.8 |
| Oxymetazoline Hydrochloride | 78.0 | 44.9 | 97.0 | 97.3 |
| Isoxsuprine Hydrochloride | 78.3 | 83.7 | 106.2 | 99.8 |
| Isoxsuprine Hydrochloride | 78.3 | 83.7 | 107.7 | 93.1 |
| Pargyline Hydrochloride | 78.6 | 105.5 | 116.9 | 86.4 |
| Nimodipine | 79.1 | 90.9 | 100.4 | 115.6 |
| Neriifolin | 79.7 | 90.3 | 110.8 | 101.2 |
| Nicotinyl Tartrate | 80.0 | 73.8 | 122.6 | 92.7 |
| Isosorbide Dinitrate | 80.5 | 66.8 | 124.6 | 100.8 |
| Pempidine Tartrate | 80.9 | 78.6 | 108.4 | 91.3 |
| 2-(2,6-Dimethoxyphenoxyethyl) | 82.6 | 94.7 | 103.9 | 95.5 |
| Aminomethyl-1,4-Benzodioxane | | | | |
| Hydrochloride | | | | |
| Phentolamine Hydrochloride | 83.4 | 72.9 | 90.7 | 110.3 |
| Disopyramide Phosphate | 83.5 | 66.3 | 125.8 | 71.2 |
| Rosuvastatin | 83.6 | 65.6 | 298.7 | 103.8 |
| Perindopril Erbumine | 83.8 | 88.8 | 72.5 | 95.3 |
| Olmesartan Medoxomil | 83.9 | 70.7 | 90.7 | 90.7 |
| Hexamethonium Bromide | 84.8 | 72.6 | 99.7 | 92.1 |
| Labetalol Hydrochloride | 84.9 | 105.5 | 164.0 | 93.0 |
| Tranexamic Acid | 84.9 | 53.9 | 112.8 | 96.8 |
| Dopamine Hydrochloride | 85.0 | 78.3 | 559.5 | 95.7 |
| Cardiovascular Drugs That Has | | | | |
| No Effect On Aβ1-40 (85-105%) | | | | |
| Triamterene | 85.1 | 64.2 | 103.3 | 93.8 |
| Bucladesine | 85.3 | 69.9 | 357.1 | 97.2 |
| Midodrine Hydrochloride | 86.0 | 76.7 | 105.6 | 87.8 |
| Phenoxybenzamine Hydrochloride | 87.5 | 90.7 | 81.3 | 72.8 |
| Pronetalol Hydrochloride | 88.0 | 94.6 | 108.9 | 88.8 |
| Fenoterol Hydrobromide | 89.3 | 78.8 | 104.3 | 93.9 |
| Trichlormethiazide | 89.3 | 84.5 | 102.6 | 105.5 |
| Warfarin | 89.4 | 107.0 | 106.6 | 100.1 |
| Hydrochlorothiazide | 89.5 | 115.6 | 281.1 | 102.1 |
| Irbesartan | 89.7 | 82.3 | 112.5 | 98.9 |
| Anisindione | 90.1 | 103.2 | 148.9 | 110.7 |
| Dobutamine Hydrochloride | 90.1 | 103.3 | 103.1 | 99.5 |
| Atenolol | 90.4 | 94.5 | 136.8 | 97.3 |
| Tolazoline Hydrochloride | 90.8 | 99.6 | 112.5 | 97.5 |
| Clofibrate | 90.9 | 92.5 | 110.5 | 97.9 |
| Telmisartan | 91.1 | 112.0 | 111.3 | 109.4 |
| Mexiletine Hydrochloride | 91.3 | 115.3 | 103.4 | 95.6 |
| Metoprolol Tartrate | 91.4 | 104.4 | 107.5 | 94.7 |
| Fenbutyramide | 91.6 | 96.3 | 135.0 | 108.6 |
| Protoveratrine A | 91.7 | 104.6 | | |
| Clopamide | 92.0 | 98.9 | 99.9 | 99.2 |
| Hydroflumethiazide | 92.4 | 90.2 | 99.7 | 98.9 |
| Urea | 92.8 | 104.9 | 130.8 | 96.7 |
| Alprenolol | 93.8 | 105.6 | 128.0 | 92.2 |
| Minoxidil | 94.2 | 98.9 | 117.1 | 98.5 |
| Nadolol | 94.2 | 113.2 | 106.5 | 102.8 |
| Fosinopril Sodium | 94.7 | 95.0 | 75.6 | 101.4 |
| Tulobuterol | 95.5 | 98.6 | 95.3 | 88.4 |
| Urapidil | 96.4 | 113.9 | 97.1 | 103.4 |
| Hydroquinidine | 96.5 | 120.7 | 102.2 | 103.6 |
| Ellagic Acid | 96.6 | 94.0 | 109.2 | 85.2 |
| Pentoxifylline | 97.1 | 109.0 | 132.4 | 91.1 |
| Pindolol | 99.0 | 46.1 | 171.7 | 98.9 |
| Vincamine | 99.1 | 104.3 | 96.4 | 98.5 |
| 2-HYDROXY-4-(2-HYDROXY-3-T- | 99.2 | 104.4 | 109.3 | 97.1 |
| BUTYLAMINOPROPOXY)- | | | | |
| BENZIMIDAZOLE | | | | |
| Practolol | 99.7 | 99.9 | 108.9 | 101.3 |
| Canrenoic Acid, Potassium Salt | 100.6 | 106.5 | 106.9 | 102.1 |
| Enalapril Maleate | 100.7 | 97.6 | 118.9 | 126.4 |
| Niacin | 101.1 | 95.0 | 134.5 | 61.0 |
| Theobromine | 101.3 | 116.7 | 118.3 | 97.2 |
| Timolol Maleate | 101.3 | 103.8 | 104.4 | 99.2 |
| Tulobuterol | 101.5 | 121.7 | 384.1 | 37.0 |
| Phenylbutyrate Sodium | 102.1 | 86.6 | 192.3 | 70.2 |
| Rauwolscine Hydrochloride | 103.2 | 74.3 | 109.4 | 98.0 |
| Phenindione | 104.0 | 104.7 | 107.9 | 108.8 |
| Metolazone | 104.0 | 98.0 | 103.7 | 69.7 |
| Captopril | 104.5 | 99.7 | 120.2 | 62.3 |
| Todralazine Hydrochloride | 104.9 | 109.8 | 100.6 | 99.5 |
| Benzthiazide | 105.7 | 98.9 | 105.1 | 98.5 |
| Cardiovascular Drugs That | | | | |
| Promote Aβ1-40 (>105%) | | | | |
| Tamsulosin Hydrchloride | 106.5 | 91.8 | 102.2 | 96.1 |
| Indapamide | 107.0 | 91.3 | 85.3 | 104.2 |
| BENZAFIBRATE | 107.2 | 99.5 | | |
| Chlorothiazide | 107.3 | 104.3 | 110.3 | 99.4 |
| Torsemide | 107.4 | 103.9 | 107.5 | 99.2 |
| Ramipril | 107.4 | 105.0 | 96.4 | 102.9 |
| Strophanthidin | 107.5 | 88.6 | 119.4 | 99.2 |
| Diltiazem Hydrochloride | 107.8 | 98.9 | 96.1 | 97.8 |
| Chlorthalidone | 108.5 | 96.7 | 645.8 | 12.1 |
| Methyldopa | 109.2 | 111.0 | 118.9 | 88.5 |
| Betahistine Hydrochloride | 110.4 | 98.9 | 107.3 | 107.1 |
| Heptaminol Hydrochloride | 112.2 | 105.4 | 103.4 | 103.4 |
| Diazoxide | 113.5 | 98.9 | 112.0 | 97.8 |
| Ajmaline | 114.0 | 103.3 | 97.6 | 90.3 |
| 1S,9R-Beta-HYDRASTINE | 114.5 | 123.6 | 114.2 | 101.1 |
| Acetazolamide | 114.7 | 118.0 | 173.5 | 78.0 |
| Peruvoside | 115.3 | 90.9 | 105.5 | 98.8 |
| Sulmazole | 115.6 | 93.2 | 111.1 | 115.0 |
| Benazepril Hydrochloride | 116.3 | 114.9 | 103.5 | 99.5 |
| Procainamide Hydrochloride | 118.1 | 108.6 | 111.1 | 96.8 |
| Molsidomine | 118.3 | 120.6 | 1127.4 | 15.0 |
| Furosemide | 118.5 | 95.7 | 98.2 | 101.0 |
| Berbamine Hydrochloride | 119.1 | 150.6 | 129.1 | 97.6 |
| Quinapril Hydrochloride | 119.3 | 118.0 | 101.8 | 102.4 |
| Pentolinium Tartrate | 121.0 | 133.9 | 118.0 | 103.8 |
| Deltaline | 121.0 | 143.0 | 118.9 | 99.4 |
| Alfluzosin | 122.7 | 66.9 | 95.4 | 91.8 |
| Aminocaproic Acid | 124.4 | 184.3 | 90.4 | 80.3 |
| Mecamylamine Hydrochloride | 124.4 | 129.6 | 97.0 | 114.9 |
| Guanethidine Sulfate | 125.5 | 149.5 | 1057.3 | 12.9 |
| Nafronyl Oxalate | 133.5 | 144.6 | 96.6 | 103.2 |
| Acebutolol Hydrochloride | 133.6 | 165.2 | 107.6 | 99.4 |
| Trandolapril | 136.7 | 110.6 | 95.5 | 91.5 |

Cardiovascular Drugs reduced Aβ1-40 were: Metergoline; Suloctidil; Bumetanide; Ethacrynic Acid; Tetrandrine; Perhexiline Maleate; Amlodipine Besylate; Bepridil Hydrochloride; Prazosin Hydrochloride; Fendiline Hydrochloride; Candesartan Cilextil; Nicardipine Hydrochloride; Fenofibrate; Amiodarone Hydrochloride; Papaverine Hydrochloride; N,N-Hexamethyleneamiloride; Reserpine; Simvastatin; Cadmium Acetate; Nitrendipine; Propafenone Hydrochloride; Carvedilol; Flunarizine Hydrochloride; Oxidopamine Hydrochloride; Lanatoside C; Lanatoside C; Dicumarol; Valsartan; Propranolol Hydrochloride (−); Veratrine Sulfate; Vinpocetine; Spironolactone; Protoveratrine B; Quinidine Gluconate; Propranolol Hydrochloride (±); Atorvastatin Calcium; Hydralazine Hydrochloride; Yohimbine Hydrochloride; Xylometazoline Hydrochloride; Digitoxin; Nylidrin Hydrochloride; Verapamil; Cyclothiazide; Chrysin; Scopoletin; Dipyridamole; Nifedipine; Althiazide; Losartan; Nicergoline; Bendrofumethiazide; Probucol; Amiloride Hydrochloride; Oxymetazoline Hydrochloride; Isoxsuprine Hydrochloride; Isoxsuprine Hydrochloride; Pargyline Hydrochloride; Nimodipine; Neriifolin; Nicotinyl Tartrate; Isosorbide Dinitrate; Pempidine Tartrate; 2-(2,6-Dimethoxyphenoxyethyl); Aminomethyl-1,4-Benzodioxane; Hydrochloride; Phentolamine Hydrochloride; Disopyramide Phosphate; Rosuvastatin; Perindopril Erbumine; Olmesartan Medoxomil; Hexamethonium Bromide; Labetalol Hydrochloride; Tranexamic Acid; Dopamine Hydrochloride.

Calcium channel blockers that may be used in the treatment of Alzheimer's Disease in accordance with the present invention include, but are not limited to: bepridil, (described in U.S. Pat. No. 3,962,238 or U.S. Reissue No. 30,577); clentiazem, (described in U.S. Pat. No. 4,567,175); diltiazem, fendiline, (see U.S. Pat. No. 3,262,977); gallopamil (described in U.S. Pat. No. 3,261,859); mibefradil (described in U.S. Pat. No. 4,808,605); prenylamine (described in U.S. Pat. No. 3,152,173); semotiadil (described in U.S. Pat. No. 4,786,635); terodiline (described in U.S. Pat. No. 3,371,014); verapamil (described in U.S. Pat. No. 3,261,859); aranipine (described in U.S. Pat. No. 4,572,909); bamidipine (described in in U.S. Pat. No. 4,220,649); benidipine (described in European Patent Application Publication No. 106,275); cilnidipine (described in U.S. Pat. No. 4,672,068); efonidipine (described in U.S. Pat. No. 4,885,284); elgodipine (described in U.S. Pat. No. 4,952,592); felodipine (described in U.S. Pat. No. 4,264,611); isradipine (described in U.S. Pat. No. 4,466,972); lacidipine (described in U.S. Pat. No. 4,801,599); lercanidipine (described in U.S. Pat. No. 4,705,797); manidipine (described in U.S. Pat. No. 4,892,875); nicardipine (described in U.S. Pat. No. 3,985,758); nifedipine (described in U.S. Pat. No. 3,485,847); nilvadipine (described in U.S. Pat. No. 4,338,322); nimodipine (described in U.S. Pat. No. 3,799,934); nisoldipine (described in U.S. Pat. No. 4,154,839); nitrendipine (described in U.S. Pat. No. 3,799,934); cinnarizine (described in U.S. Pat. No. 2,882,271); flunarzine (described in U.S. Pat. No. 3,773,939); lidoflazine (described in U.S. Pat. No. 3,267,104); lomerizine (described in U.S. Pat. No. 4,663,325); bencyclane ((described in Hungarian Patent No. 151,865); etafenone (described in German Patent No. 1,265,758); and perhexiline (described in British Patent No. 1,025,578).

Angiotensin Converting Enzyme Inhibitors (ACE-Inhibitors) which are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, which may be prepared as disclosed in U.S. Pat. Nos. 4,046,889 and 4,105,776; ceronapril, which may be prepared as disclosed in U.S. Pat. No. 4,452,790; delapril, which may be prepared as disclosed in U.S. Pat. No. 4,385,051; enalapril, which may be prepared as disclosed in U.S. Pat. No. 4,374,829; fosinopril, which may be prepared as disclosed in U.S. Pat. No. 4,337,201; imadapril, which may be prepared as disclosed in U.S. Pat. No. 4,508,727; lisinopril, which may be prepared as disclosed in U.S. Pat. No. 4,555,502; moveltopril, which may be prepared as disclosed in Belgian Patent No. 893,553; perindopril, which may be prepared as disclosed in U.S. Pat. No. 4,508,729; quinapril, which may be prepared as disclosed in U.S. Pat. No. 4,344,949; ramipril which may be prepared as disclosed in U.S. Pat. No. 4,587,258; spirapril, which may be prepared as disclosed in U.S. Pat. No. 4,470,972; temocapril, which may be prepared as disclosed in U.S. Pat. No. 4,699,905; and trandolapril, which may be prepared as disclosed in U.S. Pat. No. 4,933,361. The disclosures of all such U.S. patents are incorporated herein by reference.

Angiotensin-II receptor antagonists (A-II antagonists) are another class of agents that may be used for the treatment of Alzheimer's Disease in accordance with the present invention. Examples of such antagonists include, but are not limited to: candesartan, which may be prepared as disclosed in U.S. Pat. No. 5,196,444; eprosartan, which may be prepared as disclosed in U.S. Pat. No. 5,185,351; irbesartan, which may be prepared as disclosed in U.S. Pat. No. 5,270,317; losartan, which may be prepared as disclosed in U.S. Pat. No. 5,138,069; and valsartan, which may be prepared as disclosed in U.S. Pat. No. 5,399,578. The disclosures of all such U.S. patents are incorporated herein by reference.

Alzheimer's Disease also may be treated according to the present invention by using beta-adrenergic receptor blockers (beta- or β-blockers). Exemplary such agents known to those of skill in the art include, but are not limited to: acebutolol, which may be prepared as disclosed in U.S. Pat. No. 3,857,952; alprenolol, which may be prepared as disclosed in Netherlands Patent Application No. 6,605,692; amosulalol, which may be prepared as disclosed in U.S. Pat. No. 4,217,305; arobnolol, which may be prepared as disclosed in U.S. Pat. No. 3,932,400; atenolol, which may be prepared as disclosed in U.S. Pat. No. 3,663,607 or 3,836,671; befunolol, which may be prepared as disclosed in U.S. Pat. No. 3,853,923; betaxolol, which may be prepared as disclosed in U.S. Pat. No. 4,252,984; bevantolol, which may be prepared as disclosed in U.S. Pat. No. 3,857,981; bisoprolol, which may be prepared as disclosed in U.S. Pat. No. 4,171,370; bopindolol, which may be prepared as disclosed in U.S. Pat. No. 4,340,541; bucumolol, which may be prepared as disclosed in U.S. Pat. No. 3,663,570; bufetolol, which may be prepared as disclosed in U.S. Pat. No. 3,723,476; bufuralol, which may be prepared as disclosed in U.S. Pat. No. 3,929,836; bunitrolol, which may be prepared as disclosed in U.S. Pat. Nos. 3,940,489 and 3,961,071; buprandolol, which may be prepared as disclosed in U.S. Pat. No. 3,309,406; butiridine hydrochloride, which may be prepared as disclosed in French Patent No. 1,390,056; butofilolol, which may be prepared as disclosed in U.S. Pat. No. 4,252,825; carazolol, which may be prepared as disclosed in German Patent No. 2,240,599; carteolol, which may be prepared as disclosed in U.S. Pat. No. 3,910,924; carvedilol, which may be prepared as disclosed in U.S. Pat. No. 4,503,067; celiprolol, which may be prepared as disclosed in U.S. Pat. No. 4,034,009; cetamolol, which may be prepared as disclosed in U.S. Pat. No. 4,059,622; cloranolol, which may be prepared as disclosed in German Patent No. 2,213,044; dilevalol, which may be prepared as disclosed in Clifton et al., Journal of Medicinal Chemistry, 1982, 25, 670; epanolol, which may be prepared as disclosed in European Patent Publication Application No. 41,491; indenolol, which may be prepared as disclosed in U.S. Pat. No. 4,045,482; labetalol, which may be prepared as disclosed in U.S. Pat. No. 4,012,444; levobunolol, which may be prepared as disclosed in U.S. Pat. No. 4,463,176; mepindolol, which may be prepared as disclosed in Seeman et al., Helv. Chim. Acta, 1971, 54, 241; metipranolol, which may be prepared as disclosed in Czechoslovakian Patent Application No. 128,471; metoprolol, which may be prepared as disclosed in U.S. Pat. No. 3,873,600; moprolol, which may be prepared as disclosed in U.S. Pat. No. 3,501,7691; nadolol, which may be prepared as disclosed in U.S. Pat. No. 3,935,267; nadoxolol, which may be prepared as disclosed in U.S. Pat. No. 3,819,702; nebivalol, which may be prepared as disclosed in U.S. Pat. No. 4,654,362; nipradilol, which may be prepared as disclosed in U.S. Pat. No. 4,394,382; oxprenolol, which may be prepared as disclosed in British Patent No. 1,077,603; perbutolol, which may be prepared as disclosed in U.S. Pat. No. 3,551,493; pindolol, which may be prepared as disclosed in Swiss Patent Nos. 469,002 and 472,404; practolol, which may be prepared as disclosed in U.S. Pat. No. 3,408,387; pronethalol, which may be prepared as disclosed in British Patent No. 909,357; propranolol, which may be prepared as disclosed in U.S. Pat. Nos. 3,337,628 and 3,520,919; sotalol, which may be prepared as disclosed in Uloth et al., Journal of Medicinal Chemistry, 1966, 9, 88; sufinalol, which may be prepared as disclosed in German Patent No. 2,728,641; talindol, which may be prepared as disclosed in U.S. Pat. Nos. 3,935,259 and 4,038,313; tertatolol, which may be prepared as disclosed in U.S. Pat. No. 3,960,891; tilisolol, which may be prepared as disclosed in U.S. Pat. No. 4,129,565; timolol, which may be prepared as disclosed in U.S. Pat. No. 3,655,663; toliprolol, which may be prepared as disclosed in U.S. Pat. No. 3,432,545; and xibenolol, which may be prepared as disclosed in U.S. Pat. No. 4,018,824. The disclosures of all such U.S. patents are incorporated herein by reference.

The methods of the present invention also may be practiced by administering to a subject having Alzheimer's Disease alpha-adrenergic receptor blockers (alpha- or α-blockers) such as, for example amosulalol, which may be prepared as disclosed in U.S. Pat. No. 4,217,307; arotinolol, which may be prepared as disclosed in U.S. Pat. No. 3,932,400; dapiprazole, which may be prepared as disclosed in U.S. Pat. No. 4,252,721; doxazosin, which may be prepared as disclosed in U.S. Pat. No. 4,188,390; fenspiride, which may be prepared as disclosed in U.S. Pat. No. 3,399,192; indoramin, which may be prepared as disclosed in U.S. Pat. No. 3,527,761; labetolol, which may be prepared as disclosed above; naftopidil, which may be prepared as disclosed in U.S. Pat. No. 3,997,666; nicergoline, which may be prepared as disclosed in U.S. Pat. No. 3,228,943; prazosin, which may be prepared as disclosed in U.S. Pat. No. 3,511,836; tamsulosin, which may be prepared as disclosed in U.S. Pat. No. 4,703,063; tolazoline, which may be prepared as disclosed in U.S. Pat. No. 2,161,938; trimazosin, which may be prepared as disclosed in U.S. Pat. No. 3,669,968; and yohimbine, which may be isolated from natural sources according to methods well known to those skilled in the art. The disclosures of all such U.S. patents are incorporated herein by reference.

The cardiovascular agents used for the methods of the present invention may be vasodilators. The term “vasodilator,” where used herein, is meant to include cerebral vasodilators, coronary vasodilators and peripheral vasodilators. Cerebral vasodilators within the scope of this invention include, but are not limited to: bencyclane, which may be prepared as disclosed above; cinnarizine, which may be prepared as disclosed above; citicoline, which may be isolated from natural sources as disclosed in Kennedy et al., Journal of the American Chemical Society, 1955, 77, 250 or synthesized as disclosed in Kennedy, Journal of Biological Chemistry, 1956, 222, 185; cyclandelate, which may be prepared as disclosed in U.S. Pat. No. 3,663,597; ciclonicate, which may be prepared as disclosed in German Patent No. 1,910,481; diisopropylamine dichloroacetate, which may be prepared as disclosed in British Patent No. 862,248; ebumamonine, which may be prepared as disclosed in Hermann et al., Journal of the American Chemical Society, 1979, 101, 1540; fasudil, which may be prepared as disclosed in U.S. Pat. No. 4,678,783; fenoxedil, which may be prepared as disclosed in U.S. Pat. No. 3,818,021; flunarizine, which may be prepared as disclosed in U.S. Pat. No. 3,773,939; ibudilast, which may be prepared as disclosed in U.S. Pat. No. 3,850,941; ifenprodil, which may be prepared as disclosed in U.S. Pat. No. 3,509,164; lomerizine, which may be prepared as disclosed in U.S. Pat. No. 4,663,325; nafronyl, which may be prepared as disclosed in U.S. Pat. No. 3,334,096; nicametate, which may be prepared as disclosed in Blicke et al., Journal of the American Chemical Society, 1942, 64, 1722; nicergoline, which may be prepared as disclosed above; nimodipine, which may be prepared as disclosed in U.S. Pat. No. 3,799,934; papaverine, which may be prepared as reviewed in Goldberg, Chem. Prod. Chem. News, 1954, 17, 371; pentifylline, which may be prepared as disclosed in German Patent No. 860,217; tinofedrine, which may be prepared as disclosed in U.S. Pat. No. 3,563,997; vincamine, which may be prepared as disclosed in U.S. Pat. No. 3,770,724; vinpocetine, which may be prepared as disclosed in U.S. Pat. No. 4,035,750; and viquidil, which may be prepared as disclosed in U.S. Pat. No. 2,500,444. The disclosures of all such U.S. patents are incorporated herein by reference.

Coronary vasodilators that may be used include, but are not limited to: amotriphene, which may be prepared as disclosed in U.S. Pat. No. 3,010,965; bendazol, which may be prepared as disclosed in J. Chem. Soc. 1958, 2426; benfurodil hemisuccinate, which may be prepared as disclosed in U.S. Pat. No. 3,355,463; benziodarone, which may be prepared as disclosed in U.S. Pat. No. 3,012,042; chloracizine, which may be prepared as disclosed in British Patent No. 740,932; chromonar, which may be prepared as disclosed in U.S. Pat. No. 3,282,938; clobenfural, which may be prepared as disclosed in British Patent No. 1,160,925; clonitrate, which may be prepared from propanediol according to methods well known to those skilled in the art, e.g., see Annalen, 1870, 155, 165; cloricromen, which may be prepared as disclosed in U.S. Pat. No. 4,452,811; dilazep, which may be prepared as disclosed in U.S. Pat. No. 3,532,685; dipyridamole, which may be prepared as disclosed in British Patent No. 807,826; droprenilamine, which may be prepared as disclosed in German Patent No. 2,521,113; efloxate, which may be prepared as disclosed in British Patent Nos. 803,372 and 824,547; erythrityl tetranitrate, which may be prepared by nitration of erythritol according to methods well-known to those skilled in the art; etafenone, which may be prepared as disclosed in German Patent No. 1,265,758; fendiline, which may be prepared as disclosed in U.S. Pat. No. 3,262,977; floredil, which may be prepared as disclosed in German Patent No. 2,020,464; ganglefene, which may be prepared as disclosed in U.S.S.R. Patent No. 115,905; hexestrol, which may be prepared as disclosed in U.S. Pat. No. 2,357,985; hexobendine, which may be prepared as disclosed in U.S. Pat. No. 3,267,103; itramin tosylate, which may be prepared as disclosed in Swedish Patent No. 168,308; khellin, which may be prepared as disclosed in Baxter et al., Journal of the Chemical Society, 1949, S 30; lidoflazine, which may be prepared as disclosed in U.S. Pat. No. 3,267,104; mannitol hexanitrate, which may be prepared by the nitration of mannitol according to methods well-known to those skilled in the art; medibazine, which may be prepared as disclosed in U.S. Pat. No. 3,119,826; nitroglycerin; pentaerythritol tetranitrate, which may be prepared by the nitration of pentaerythritol according to methods well-known to those skilled in the art; pentrinitrol, which may be prepared as disclosed in German Patent No. 638,422-3; perhexilline, which may be prepared as disclosed above; pimethylline, which may be prepared as disclosed in U.S. Pat. No. 3,350,400; prenylamine, which may be prepared as disclosed in U.S. Pat. No. 3,152,173; propatyl nitrate, which may be prepared as disclosed in French Patent No. 1,103,113; trapidil, which may be prepared as disclosed in East German Patent No. 55,956; tricromyl, which may be prepared as disclosed in U.S. Pat. No. 2,769,015; trimetazidine, which may be prepared as disclosed in U.S. Pat. No. 3,262,852; trolnitrate phosphate, which may be prepared by nitration of triethanolamine followed by precipitation with phosphoric acid according to methods well-known to those skilled in the art; visnadine, which may be prepared as disclosed in U.S. Pat. Nos. 2,816,118 and 2,980,699. The disclosures of all such U.S. patents are incorporated herein by reference.

Peripheral vasodilators that may be used as cardiovascular agents in the scope of the present invention include, but are not limited to: aluminum nicotinate, which may be prepared as disclosed in U.S. Pat. No. 2,970,082; bamethan, which may be prepared as disclosed in Corrigan et al., Journal of the American Chemical Society, 1945, 67, 1894; bencyclane, which may be prepared as disclosed above; betahistine, which may be prepared as disclosed in Walter et al.; Journal of the American Chemical Society, 1941, 63, 2771; bradykinin, which may be prepared as disclosed in Hamburg et al., Arch. Biochem. Biophys., 1958, 76, 252; brovincamine, which may be prepared as disclosed in U.S. Pat. No. 4,146,643; bufeniode, which may be prepared as disclosed in U.S. Pat. No. 3,542,870; buflomedil, which may be prepared as disclosed in U.S. Pat. No. 3,895,030; butalamine, which may be prepared as disclosed in U.S. Pat. No. 3,338,899; cetiedil, which may be prepared as disclosed in French Patent Nos. 1,460,571; ciclonicate, which may be prepared as disclosed in German Patent No. 1,910,481; cinepazide, which may be prepared as disclosed in Belgian Patent No. 730,345; cinnarizine, which may be prepared as disclosed above; cyclandelate, which may be prepared as disclosed above; diisopropylamine dichloroacetate, which may be prepared as disclosed above; eledoisin, which may be prepared as disclosed in British Patent No. 984,810; fenoxedil, which may be prepared as disclosed above; flunarizine, which may be prepared as disclosed above; hepronicate, which may be prepared as disclosed in U.S. Pat. No. 3,384,642; ifenprodil, which may be prepared as disclosed above; iloprost, which may be prepared as disclosed in U.S. Pat. No. 4,692,464; inositol niacinate, which may be prepared as disclosed in Badgett et al., Journal of the American Chemical Society, 1947, 69, 2907; isoxsuprine, which may be prepared as disclosed in U.S. Pat. No. 3,056,836; kallidin, which may be prepared as disclosed in Biochem. Biophys. Res. Commun., 1961, 6, 210; kallikrein, which may be prepared as disclosed in German Patent No. 1,102,973; moxisylyte, which may be prepared as disclosed in German Patent No. 905,738; nafronyl, which may be prepared as disclosed above; nicametate, which may be prepared as disclosed above; nicergoline, which may be prepared as disclosed above; nicofuranose, which may be prepared as disclosed in Swiss Patent No. 366,523; nylidrin, which may be prepared as disclosed in U.S. Pat. Nos. 2,661,372 and 2,661,373; pentifylline, which may be prepared as disclosed above; pentoxifylline, which may be prepared as disclosed in U.S. Pat. No. 3,422,107; piribedil, which may be prepared as disclosed in U.S. Pat. No. 3,299,067; prostaglandin E₁, which may be prepared by any of the methods referenced in the Merck Index, Twelfth Edition, Budaveri, Ed., New Jersey, 1996, p. 1353; suloctidil, which may be prepared as disclosed in German Patent No. 2,334,404; tolazoline, which may be prepared as disclosed in U.S. Pat. No. 2,161,938; and xanthinol niacinate, which may be prepared as disclosed in German Patent No. 1,102,750 or Korbonits et al., Acta. Pharm. Hung., 1968, 38, 98. The disclosures of all such U.S. patents are incorporated herein by reference.

Diuretic agents also are known to be used as antihypertensive cardiovascular agents and it is contemplated that such antihyoertensive diuretic agents may be used in the methods of the present invention. Diuretic agents within the scope of this invention includes any diuretic agent that will produce an antihypertensive cardiovascular effect. Such agents include, for example, diuretic benzothiadiazine derivatives, diuretic organomercurials, diuretic purines, diuretic steroids, diuretic sulfonamide derivatives, diuretic uracils and other diuretics such as amanozine, which may be prepared as disclosed in Austrian Patent No. 168,063; amiloride, which may be prepared as disclosed in Belgian Patent No. 639,386; arbutin, which may be prepared as disclosed in Tschitschibabin, Annalen, 1930, 479, 303; chlorazanil, which may be prepared as disclosed in Austrian Patent No. 168,063; ethacrynic acid, which may be prepared as disclosed in U.S. Pat. No. 3,255,241; etozolin, which may be prepared as disclosed in U.S. Pat. No. 3,072,653; hydracarbazine, which may be prepared as disclosed in British Patent No. 856,409; isosorbide, which may be prepared as disclosed in U.S. Pat. No. 3,160,641; mannitol; metochaloone, which may be prepared as disclosed in Freudenberg et al., Ber., 1957, 90, 957; muzolimine, which may be prepared as disclosed in U.S. Pat. No. 4,018,890; perhexiline, which may be prepared as disclosed above; ticrynafen, which may be prepared as disclosed in U.S. Pat. No. 3,758,506; triamterene which may be prepared as disclosed in U.S. Pat. No. 3,081,230; and urea. The disclosures of all such U.S. patents are incorporated herein by reference.

Exemplary diuretic benzothiadiazine derivatives for use herein include for example: althiazide, which may be prepared as disclosed in British Patent No. 902,658; bendroflumethiazide, which may be prepared as disclosed in U.S. Pat. No. 3,265,573; benzthiazide, McManus et al., 136th Am. Soc. Meeting (Atlantic City, September 1959), Abstract of papers, pp 13-O; benzylhydrochlorothiazide, which may be prepared as disclosed in U.S. Pat. No. 3,108,097; buthiazide, which may be prepared as disclosed in British Patent Nos. 861,367 and 885,078; chlorothiazide, which may be prepared as disclosed in U.S. Pat. Nos. 2,809,194 and 2,937,169; chlorthalidone, which may be prepared as disclosed in U.S. Pat. No. 3,055,904; cyclopenthiazide, which may be prepared as disclosed in Belgian Patent No. 587,225; cyclothiazide, which may be prepared as disclosed in Whitehead et al., Journal of Organic Chemistry, 1961, 26, 2814; epithiazide, which may be prepared as disclosed in U.S. Pat. No. 3,009,911; ethiazide, which may be prepared as disclosed in British Patent No. 861,367; fenquizone, which may be prepared as disclosed in U.S. Pat. No. 3,870,720; indapamide, which may be prepared as disclosed in U.S. Pat. No. 3,565,911; hydrochlorothiazide, which may be prepared as disclosed in U.S. Pat. No. 3,164,588; hydroflumethiazide, which may be prepared as disclosed in U.S. Pat. No. 3,254,076; methyclothiazide, which may be prepared as disclosed in Close et al., Journal of the American Chemical Society, 1960, 82, 1132; meticrane, which may be prepared as disclosed in French Patent Nos. M2790 and 1,365,504; metolazone, which may be prepared as disclosed in U.S. Pat. No. 3,360,518; paraflutzide, which may be prepared as disclosed in Belgian Patent No. 620,829; polythiazide, which may be prepared as disclosed in U.S. Pat. No. 3,009,911; quinethazone, which may be prepared as disclosed in U.S. Pat. No. 2,976,289; teclothiazide, which may be prepared as disclosed in Close et al., Journal of the American Chemical Society, 1960, 82, 1132; and trichlormethiazide, which may be prepared as disclosed in deStevens et al., Experientia, 1960, 16, 113. The disclosures of all such U.S. patents are incorporated herein by reference.

Examples of diuretic sulfonamide derivatives that may be used in the present invention include, but are not limited to: acetazolamide, which may be prepared as disclosed in U.S. Pat. No. 2,980,679; ambuside, which may be prepared as disclosed in U.S. Pat. No. 3,188,329; azosemide, which may be prepared as disclosed in U.S. Pat. No. 3,665,002; bumetamide, which may be prepared as disclosed in U.S. Pat. No. 3,634,583; butazolamide, which may be prepared as disclosed in British Patent No. 769,757; chloraminophenamide, which may be prepared as disclosed in U.S. Pat. Nos. 2,809,194, 2,965,655 and 2,965,656; clofenamide, which may be prepared as disclosed in Olivier, Rec. Trav. Chim., 1918, 37, 307; clopamide, which may be prepared as disclosed in U.S. Pat. No. 3,459,756; clorexolone, which may be prepared as disclosed in U.S. Pat. No. 3,183,243; disulfamide, which may be prepared as disclosed in British Patent No. 851,287; ethoxolamide, which may be prepared as disclosed in British Patent No. 795,174; furosemide, which may be prepared as disclosed in U.S. Pat. No. 3,058,882; mefruside, which may be prepared as disclosed in U.S. Pat. No. 3,356,692; methazolamide, which may be prepared as disclosed in U.S. Pat. No. 2,783,241; piretanide, which may be prepared as disclosed in U.S. Pat. No. 4,010,273; torasemide, which may be prepared as disclosed in U.S. Pat. No. 4,018,929; tripamide, which may be prepared as disclosed in Japanese Patent No. 73, 05,585; and xipamide, which may be prepared as disclosed in U.S. Pat. No. 3,567,777. The disclosures of all such U.S. patents are incorporated herein by reference.

The present invention will use cardiovascular compounds, and pharmaceutically acceptable salts thereof, for treating humans and/or animals suffering from a condition characterized by a pathological form of beta-amyloid peptide, such as beta-amyloid plaques, and for helping to prevent or delay the onset of such a condition. For example, the cardiovascular compounds can be used to treat Alzheimer's disease, to help prevent or delay the onset of Alzheimer's disease, to treat patients with MCI (mild cognitive impairment) and prevent or delay the onset of Alzheimer's disease in those who would progress from MCI to AD, to treat Down's syndrome, to treat humans who have Hereditary Cerebral Hemorrhage with Amyloidosis of the Dutch-Type, to treat cerebral amyloid angiopathy and prevent its potential consequences, i.e. single and recurrent lobal hemorrhages, to treat dementia associated with cortical basal degeneration, and diffuse Lewy body type Alzheimer's disease. It has been discovered herein that standard cardiovascular agents and compositions are particularly suitable for treating or preventing Alzheimer's disease. When treating or preventing these diseases, the cardiovascular compounds can either be used individually or in combination, as is best for the patient. The cardiovascular agents also can be used in combination with other anti-Alzheimer's disease therapies.

As used herein, the term “treating” means that the cardiovascular agents can be used in humans with at least a tentative diagnosis of Alzheimer's disease. The cardiovascular agents will delay or slow the progression of the disease thereby giving the individual a more useful life span.

The term “preventing” means that the cardiovascular agents are administered to a patient who has not been diagnosed as possibly having the disease at the time of administration, but who would normally be expected to develop the disease or be at increased risk for the disease. The cardiovascular agents used in the inventive methods of the invention will slow the development of disease symptoms, delay the onset of the disease, or prevent the individual from developing the disease at all. Preventing also includes administration of the compounds to those individuals thought to be predisposed to the disease due to age, familial history, genetic or chromosomal abnormalities, and/or due to the presence of one or more biological markers for the disease, such as a known genetic mutation of APP or APP cleavage products in brain tissues or fluids.

In treating or preventing the above diseases, the cardiovascular compounds are administered in a therapeutically effective amount. The therapeutically effective amount will vary depending on the particular compound used and the route of administration, as is known to those skilled in the art. However, as noted above, the cardiovascular compounds are all commercially available and well-tolerated in patients with hypertension. Hence doses of the agents that are typically used in such hypertension indications will be used in the methods of the present invention.

In treating a patient displaying any of the above diagnosed conditions a physician may administer an cardiovascular compound immediately and continue administration indefinitely, as needed. In treating patients who are not diagnosed as having Alzheimer's disease, but who are believed to be at substantial risk for Alzheimer's disease, the physician should preferably start treatment when the patient first experiences early pre-Alzheimer's symptoms such as, memory or cognitive problems associated with aging. In addition, there are some patients who may be determined to be at risk for developing Alzheimer's through the detection of a genetic marker such as APOE4 or other biological indicators that are predictive for Alzheimer's disease. In these situations, even though the patient does not have symptoms of the disease, administration of the cardiovascular agents may be started before symptoms appear, and treatment may be continued indefinitely to prevent or delay the outset of the disease.

The cardiovascular agents may be administered orally, parentemally, (IV, IM, depo-IM, SQ, and depo SQ), sublingually, intranasally (inhalation), intrathecally, topically, or rectally. Dosage forms known to those of skill in the art are suitable for delivery of the cardiovascular compounds.

In the present invention, the cardiovascular compositions are provided in therapeutically effective amounts, preferably formulated into suitable pharmaceutical preparations such as tablets, capsules, or elixirs for oral administration or in sterile solutions or suspensions for parenternal administration. Typically the compounds described above are formulated into pharmaceutical compositions using techniques and procedures well known in the art.

About 1 to 500 mg of a compound or mixture of cardiovascular agents or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more therapeutic compounds are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

In certain embodiments, valsartan is used as an example of the agents of the invention. Valsartan was converted into a sodium salt to improve the solubility of valsartan in aqueous solutions. Sodium valsartan was prepared by dissolving and mixing equimolar amounts of valsartan and sodium chloride in methanol and then drying the solution under high vacuum to constant weight. Since sodium valsartan is hygroscopic, it is stored in a dry and dark environment until used. Sodium valsartan does not have any labile groups and we found valsartan to be stable in both the solid and the aqueous state. An aqueous valsartan solution is prepared by adding valsartan salt to water at 30-40° C. and stirred vigorously until valsartan is completely dissolved. The solution is cooled to room temperature slowly without external cooling to discourage precipitation of the drug. Sodium valsartan has a solubility of ˜5 g/L at room temperature, and the aqueous valsartan salt solution is slightly acidic, with a pH of 5.5. We generally neutralize aqueous valsartan solution with sodium bicarbonate without detectable reduction of valsartan solubility. For our preclinical studies, we prepared neutralized valsartan aqueous solutions at concentrations (50-200 mg/L) well below the maximal solubility of sodium valsartan in water. Neutralized valsartan solutions are routinely stored at room temperature in a dark environment to minimize potential precipitation of the compound from the solution or photochemical changes Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The cardiovascular agents may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutic effect in the absence of undesirable side effects on the patient treated. The therapeutically effective concentration may be determined empirically by testing the compounds in known in vitro and in vivo model systems for the treated disorder.

The compounds and compositions can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the cardiovascular agent. The containers are preferably adapted for the desired mode of administration, including, but not limited to tablets, gel capsules, sustained-release capsules, and the like for oral administration; depot products, pre-filled syringes, ampules, vials, and the like for parentemal administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The oral dosage forms are administered to the patient 1, 2, 3, or 4 times daily. It is preferred that the cardiovascular agent be administered either three or fewer times, more preferably once or twice daily. Hence, it is preferred that the cardiovascular agent be administered in oral dosage form. It is preferred that whatever oral dosage form is used, that it be designed so as to protect the cardiovascular agent from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to inhibit beta-secretase activity, to inhibit A beta production, to inhibit A beta deposition, or to treat or prevent AD is from about 0.1 mg/day to about 1,000 mg/day. It is preferred that the oral dosage is from about 1 mg/day to about 100 mg/day. It is more preferred that the oral dosage is from about 5 mg/day to about 50 mg/day. It is understood that while a patient may be started at one dose, that dose may be varied over time as the patient's condition changes.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

Solutions or suspensions used for parenternal, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenternal preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

When administered parenterally, a therapeutically effective amount of about 0.5 to about 100 mg/day, preferably from about 5 to about 50 mg daily should be delivered. When a depot formulation is used for injection once a month or once every two weeks, the dose should be about 0.5 mg/day to about 50 mg/day, or a monthly dose of from about 15 mg to about 1,500 mg. In part because of the forgetfulness of the patients with Alzheimer's disease, it is preferred that the parenteral dosage form be a depo formulation.

The cardiovascular compounds can be administered intrathecally. When given by this route the appropriate dosage form can be a parenternal dosage form as is known to those skilled in the art. The dosage of the cardiovascular compounds for intrathecal administration is the amount described above for IM administration.

The cardiovascular compounds can be administered topically. When given by this route, the appropriate dosage form is a cream, ointment, or patch. Because of the amount of the cardiovascular compound to be administered, the patch is preferred. When administered topically, the dosage is from about 0.5 mg/day to about 200 mg/day. Because the amount that can be delivered by a patch is limited, two or more patches may be used. The number and size of the patch is not important, what is important is that a therapeutically effective amount of the cardiovascular compound be delivered as is known to those skilled in the art. The cardiovascular compound can be administered rectally by suppository as is known to those skilled in the art. When administered by suppository, the therapeutically effective amount is from about 0.5 mg to about 500 mg.

The compounds of the invention can be administered by implants as is known to those skilled in the art. When administering a compound of the invention by implant, the therapeutically effective amount is the amount described above for depot administration.

The cardiovascular compound may be in the same manner, by the same routes of administration, using the same pharmaceutical dosage forms, and at the same dosing schedule as described above, for preventing disease or treating patients with MCI (mild cognitive impairment) and preventing or delaying the onset of Alzheimer's disease in those who would progress from MCI to AD, for treating or preventing Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type of Alzheimer's disease.

The cardiovascular compounds can be used in combination, with each other or with other therapeutic agents or approaches used to treat or prevent the conditions listed above. Such agents or approaches include: acetylcholine esterase inhibitors such as tacrine (tetrahydroaminoacridine, marketed as COGNEX®), donepezil hydrochloride, (marketed as Aricept® and rivastigmine (marketed as Exelon®); gamma-secretase inhibitors; anti-inflammatory agents such as cyclooxygenase II inhibitors; anti-oxidants such as Vitamin E and ginkolides; immunological approaches, such as, for example, immunization with A beta peptide or administration of anti-A beta peptide antibodies; statins; and direct or indirect neurotropic agents such as Cerebrolysin®, AIT-082 (Emilieu, 2000, Arch. Neurol. 57:454), and other neurotropic agents of the future.

It should be apparent to one skilled in the art that the exact dosage and frequency of administration will depend on the particular compounds being administered, the particular condition being treated, the severity of the condition being treated, the age, weight, general physical condition of the particular patient, and other medication the individual may be taking as is well known to administering physicians who are skilled in this art.

As noted above, cardiovascular agents are used in order to treat AD. Such agents have an AB lowering activity. The cardiovascular compound may do this by inhibiting the cleavage of APP, inhibiting production of AB peptide, inhibiting cleavage of AB peptide of increasing the egress of the AB peptide from the brain cells. While not wishing to be bound by a particular theory, inhibition these therapeutic effects of the cardiovascular agents ultimately inhibit production of beta amyloid peptide (A beta). Inhibitory activity of cardiovascular agents may be tested in one of a variety of inhibition assays, whereby cleavage of an APP substrate in the presence of a beta-secretase enzyme is analyzed in the presence of the cardiovascular compound, under conditions normally sufficient to result in cleavage at the beta-secretase cleavage site. Reduction of APP cleavage at the beta-secretase cleavage site compared with an untreated or inactive control is correlated with inhibitory activity of the cardiovascular agent. In this manner any cardiovascular agent can be effectively screened. Assay systems that can be used to demonstrate efficacy of the cardiovascular agents are known. Representative assay systems are described, for example, in U.S. Pat. Nos. 5,942,400, 5,744,346, as well as in the Examples below.

The enzymatic activity of beta-secretase and the production of A beta can be analyzed in vitro or in vivo, using natural, mutated, and/or synthetic APP substrates, natural, mutated, and/or synthetic enzyme, and the test compound. The analysis may involve primary or secondary cells expressing native, mutant, and/or synthetic APP and enzyme, animal models expressing native APP and enzyme, or may utilize transgenic animal models expressing the substrate and enzyme. Detection of enzymatic activity can be by analysis of one or more of the cleavage products, for example, by immunoassay, flurometric or chromogenic assay, HPLC, or other means of detection. Inhibitory compounds are determined as those having the ability to decrease the amount of beta-secretase cleavage product produced in comparison to a control, where beta-secretase mediated cleavage in the reaction system is observed and measured in the absence of inhibitory compounds.

Various forms of beta-secretase enzyme are known, and are available for assay of enzyme activity and inhibition of enzyme activity. These include native, recombinant, and synthetic forms of the enzyme. Human beta-secretase is known as Beta Site APP Cleaving Enzyme (BACE), Asp2, and memapsin 2, and has been characterized, for example, in U.S. Pat. No. 5,744,346 and published PCT patent applications WO98/22597, WO00/03819, WO01/23533, and WO00/17369, as well as in literature publications (Hussain et. al., 1999, Mol. Cell. Neurosci. 14:419-427; Vassar et. al., 1999, Science 286:735-741; Yan et. al., 1999, Nature 402:533-537; Sinha et. al., 1999, Nature 40:537-540; and Lin et. al., 2000, PNAS USA 97:1456-1460). Synthetic forms of the enzyme have also been described (WO98/22597 and WO00/17369). Beta-secretase can be extracted and purified from human brain tissue and can be produced in cells, for example mammalian cells expressing recombinant enzyme.

Preferred cardiovascular agents will be those that are effective to inhibit 50% of beta-secretase enzymatic activity at a concentration of less than 50 micromolar, preferably at a concentration of 10 micromolar or less, more preferably 1 micromolar or less, and most preferably 10 nanomolar or less. Alternatively, such agents are effective to inhibit 50% of of the production of AB peptide at a concentration of less than 50 micromolar, preferably at a concentration of 10 micromolar or less, more preferably 1 micromolar or less, and most preferably 10 nanomolar or less. In still other embodiments, the agents increase the egress of AB from the brain by at least 25%, preferably 50% as compared to the egress in the absence of such cardiovascular agent.

Assays that demonstrate inhibition of beta-secretase-mediated cleavage of APP can utilize any of the known forms of APP, including the 695 amino acid “normal” isotype described by Kang et. al., 1987, Nature 325:733-6, the 770 amino acid isotype described by Kitaguchi et. al., 1981, Nature 331:530-532, and variants such as the Swedish Mutation (KM670-1NL) (APP-SW), the London Mutation (V7176F), and others. See, for example, U.S. Pat. No. 5,766,846 and also Hardy, 1992, Nature Genet. 1:233-234, for a review of known variant mutations. Additional substrates include the dibasic amino acid modification, APP-KK disclosed, for example, in WO 00/17369, fragments of APP, and synthetic peptides containing the beta-secretase cleavage site, wild type (WT) or mutated form, e.g., SW, as described, for example, in U.S. Pat. No. 5,942,400 and WO00/03819.

The APP substrate contains the beta-secretase cleavage site of APP (KM-DA or NL-DA) for example, a complete APP peptide or variant, an APP fragment, a recombinant or synthetic APP, or a fusion peptide. Preferably, the fusion peptide includes the beta-secretase cleavage site fused to a peptide having a moiety useful for enzymatic assay, for example, having isolation and/or detection properties. Such moieties include, for example, an antigenic epitope for antibody binding, a label or other detection moiety, a binding substrate, and the like.

Assays for determining APP cleavage at the beta-secretase cleavage site are well known in the art. Exemplary assays, are described, for example, in U.S. Pat. Nos. 5,744,346 and 5,942,400, and described in the Examples below.

Exemplary assays that can be used to demonstrate the inhibitory activity of cardiovascular agents in an Alzheimer's Disease phenotype are described, for example, in WO00/17369, WO 00/03819, and U.S. Pat. Nos. 5,942,400 and 5,744,346. Such assays can be performed in cell-free incubations or in cellular incubations using cells expressing a beta-secretase and an APP substrate having a beta-secretase cleavage site.

An APP substrate containing the beta-secretase cleavage site of APP, for example, a complete APP or variant, an APP fragment, or a recombinant or synthetic APP substrate containing the amino acid sequence: KM-DA or NL-DA, is incubated in the presence of beta-secretase enzyme, a fragment thereof, or a synthetic or recombinant polypeptide variant having beta-secretase activity and effective to cleave the beta-secretase cleavage site of APP, under incubation conditions suitable for the cleavage activity of the enzyme. Suitable substrates optionally include derivatives that may be fusion proteins or peptides that contain the substrate peptide and a modification to facilitate the purification or detection of the peptide or its beta-secretase cleavage products. Modifications include the insertion of a known antigenic epitope for antibody binding; the linking of a label or detectable moiety, the linking of a binding substrate, and the like.

Suitable incubation conditions for a cell-free in vitro assay include, for example: approximately 200 nanomolar to 10 micromolar substrate, approximately 10 to 200 picomolar enzyme, and approximately 0.1 nanomolar to 10 micromolar inhibitor compound, in aqueous solution, at an approximate pH of 4-7, at approximately 37 degrees C., for a time period of approximately 10 minutes to 3 hours. These incubation conditions are exemplary only, and can be varied as required for the particular assay components and/or desired measurement system. Optimization of the incubation conditions for the particular assay components should account for the specific beta-secretase enzyme used and its pH optimum, any additional enzymes and/or markers that might be used in the assay, and the like. Such optimization is routine and will not require undue experimentation.

One assay utilizes a fusion peptide having maltose binding protein (MBP) fused to the C-terminal 125 amino acids of APP-SW. The MBP portion is captured on an assay substrate by anti-MBP capture antibody. Incubation of the captured fusion protein in the presence of beta-secretase results in cleavage of the substrate at the beta-secretase cleavage site. Analysis of the cleavage activity can be, for example, by immunoassay of cleavage products. One such immunoassay detects a unique epitope exposed at the carboxy terminus of the cleaved fusion protein, for example, using the antibody SW192. This assay is described, for example, in U.S. Pat. No. 5,942,400.

Numerous cell-based assays can be used to analyze beta-secretase activity and/or processing of APP to release A beta. Contact of an APP substrate with a beta-secretase enzyme within the cell and in the presence or absence of an cardiovascular compound can be used to demonstrate beta-secretase inhibitory activity of the compound. Preferably, assay in the presence of an inhibitory compound provides at least about 30%, most preferably at least about 50% inhibition of the enzymatic activity, as compared with a non-inhibited control.

In one embodiment, cells that naturally express beta-secretase are used. Alternatively, cells are modified to express a recombinant beta-secretase or synthetic variant enzyme as discussed above. The APP substrate may be added to the culture medium and is preferably expressed in the cells. Cells that naturally express APP, variant or mutant forms of APP, or cells transformed to express an isoform of APP, mutant or variant APP, recombinant or synthetic APP, APP fragment, or synthetic APP peptide or fusion protein containing the beta-secretase APP cleavage site can be used, provided that the expressed APP is permitted to contact the enzyme and enzymatic cleavage activity can be analyzed.

Human cell lines that normally process A beta from APP provide a means to assay inhibitory activities of the cardiovascular compounds. Production and release of A beta and/or other cleavage products into the culture medium can be measured, for example by immunoassay, such as Western blot or enzyme-linked immunoassay (EIA) such as by ELISA.

Cells expressing an APP substrate and an active beta-secretase can be incubated in the presence of a compound inhibitor to demonstrate inhibition of enzymatic activity as compared with a control. Activity of beta-secretase can be measured by analysis of one or more cleavage products of the APP substrate. For example, inhibition of beta-secretase activity against the substrate APP would be expected to decrease release of specific beta-secretase induced APP cleavage products such as A beta.

Although both neural and non-neural cells process and release A beta, levels of endogenous beta-secretase activity are low and often difficult to detect by ELISA. The use of cell types known to have enhanced beta-secretase activity, enhanced processing of APP to A beta, and/or enhanced production of A beta are therefore preferred. For example, transfection of cells with the Swedish Mutant form of APP (APP-SW); with APP-KK; or with APP-SW-KK provides cells having enhanced beta-secretase activity and producing amounts of A beta that can be readily measured.

In such assays, for example, the cells expressing APP and beta-secretase are incubated in a culture medium under conditions suitable for beta-secretase enzymatic activity at its cleavage site on the APP substrate. On exposure of the cells to the compound inhibitor, the amount of A beta released into the medium and/or the amount of CTF99 fragments of APP in the cell lysates is reduced as compared with the control. The cleavage products of APP can be analyzed, for example, by immune reactions with specific antibodies, as discussed above.

Preferred cells for analysis of beta-secretase activity include primary human neuronal cells, primary transgenic animal neuronal cells where the transgene is APP, and other cells such as those of a stable 293 cell line expressing APP, for example, APP-SW.

Various animal models can be used to analyze beta-secretase activity and/or processing of APP to release A beta, as described above. For example, transgenic animals expressing APP substrate and beta-secretase enzyme can be used to demonstrate inhibitory activity of the cardiovascular compounds. Certain transgenic animal models have been described, for example, in U.S. Pat. Nos. 5,877,399; 5,612,486; 5,387,742; 5,720,936; 5,850,003; 5,877,015, and 5,811,633, and in Ganes et. al., 1995, Nature 373:523. Preferred are animals that exhibit characteristics associated with the pathophysiology of AD. Administration of the cardiovascular compound to the transgenic mice described herein provides an alternative method for demonstrating the inhibitory activity of the compound. Administration of the compounds in a pharmaceutically effective carrier and via an administrative route that reaches the target tissue in an appropriate therapeutic amount is also preferred.

Inhibition of beta-secretase mediated cleavage of APP at the beta-secretase cleavage site and of A beta release can be analyzed in these animals by measure of cleavage fragments in the animal's body fluids such as cerebral fluid or tissues. Analysis of brain tissues for A beta deposits or plaques is preferred.

On contacting an APP substrate with a beta-secretase enzyme in the presence of an cardiovascular compound and under conditions sufficient to permit enzymatic mediated cleavage of APP and/or release of A beta from the substrate, the cardiovascular compounds are effective to reduce beta-secretase-mediated cleavage of APP at the beta-secretase cleavage site and/or effective to reduce released amounts of A beta. Where such contacting is the administration of the cardiovascular agent to an animal model, for example, as described above, the compounds are effective to reduce A beta deposition in brain tissues of the animal, and to reduce the number and/or size of beta amyloid plaques. Where such administration is to a human subject, the compounds are effective to inhibit or slow the progression of disease characterized by enhanced amounts of A beta, to slow the progression of AD in the, and/or to prevent onset or development of AD in a patient at risk for the disease.

Patients that have Alzheimer's Disease (AD) demonstrate an increased amount of A beta in the brain. AD patients are administered an amount of an cardiovascular agent formulated in a carrier suitable for the chosen mode of administration. Administration is repeated daily for the duration of the test period. Beginning on day 0, cognitive and memory tests are performed, for example, once per month.

Patients that are administered cardiovascular agents are expected to demonstrate slowing or stabilization of disease progression as analyzed by changes in one or more of the following disease parameters: A beta present in CSF or plasma; brain or hippocampal volume; A beta deposits in the brain; amyloid plaque in the brain; and scores for cognitive and memory function, as compared with control, non-treated patients.

Patients that are predisposed or at risk for developing AD are identified either by recognition of a familial inheritance pattern, for example, presence of the Swedish Mutation, and/or by monitoring diagnostic parameters. Patients identified as predisposed or at risk for developing AD are administered an amount of the compound inhibitor formulated in a carrier suitable for the chosen mode of administration. Administration is repeated daily for the duration of the test period. Beginning on day 0, cognitive and memory tests are performed, for example, once per month.

Patients that are given administered cardiovascular agents are expected to demonstrate slowing or stabilization of disease progression as analyzed by changes in one or more of the following disease parameters: A beta present in CSF or plasma; brain or hippocampal volume; amyloid plaque in the brain; and scores for cognitive and memory function, as compared with control, non-treated patients.

### Examples

### Example 1

### Initial Investigations

### Identification of Aβ-Lowering Activities in Commonly Prescribed Cardiovascular Agents

The present invention was based on an exploration of the potential Aβ-lowering activity of 150 commercially available cardiovascular agents. The compounds tested represent a wide spectrum of pharmacological profiles, one of which is antihypertensive activity. Initially, 57 cardiovascular agents were identified as being capable of significantly reducing Aβ₁₋₄₀ and/or Aβ₁₋₄₂ generation (by ≧15%) in primary cortico-hippocampal neuron cultures generated from Tg2576 AD mice, a well-recognized model of AD.

Based on the initial results, further studies were conducted to assess the “secondary dose-response screening” of the 57 candidate agents for Aβ-lowering activity. The effective concentrations of agents resulting in a 50% inhibition (EC50) of Aβ₁₋₄₀ and for Aβ₁₋₄₂ content in the conditioning medium of the neuron cultures were calculated, relative to parallel vehicle-treated Tg2576 control cultures. Excitingly, 24 “cardiovascular drugs” were identified which exerted dose-dependent Aβ₁₋₄₀ and/or Aβ₁₋₄₂ lowering activity with a predicted EC50 at ≦10 μM (Table I). No apparent neurotoxicity was associated with any of the agents, as assessed by a lactate dehydrogenase (LDH) activity assay in parallel cultures at identical drug concentrations (Table 1).

It was found that with the exception of “anti-arrhythmics” (n=2)” and “anti-coagulants” (n=1), 21 of the 24 identified candidate agents had pharmacological antihypertensive properties (Table I). This evidence is of great interest and provides support for the use of antihypertensives to influence Aβ generation/clearance from the brain, ultimately resulting in prevention of AD amyloid neuropathology.

Table 1 presented below provides identification of candidate cardiovascular drugs with Aβ-lowering activity in vitro. Potential Aβ-lowering activity was assessed for 150 commercially available cardiovascular drugs using primary cortico-hippocampal neuron cultures derived from embryonic Tg2576 (El 6). Cultures were maintained in a serum-free Neurobasal medium in the presence of L-glutamine and B27 supplement as described in Mirjany et al (2002). All cardiovascular reagents were obtained from MicroSource Discovery Systems Inc (Gaylordsville, Conn.). The Spectrum Collection contains biologically active and structurally diverse compounds of known drugs as a stock 10 mM concentration in DMSO. Individual cardiovascular agents were applied directly to the culture medium (final 1% DMSO in the culture media). In control studies, parallel Tg2576 primary neuron cultures were treated with vehicle (1% DMSO) alone. The influence of drug treatments in Aβ content in the conditioned medium in treated cultures was assessed by assessing steady state levels of Aβ₁₋₄₀ and Aβ₁₋₄₂ in the culture media 24 hours after treatment. Aβ peptides contents were quantified using ELSIA assays as previously discussed (Appendix 3; Wang et al, 2005). In the primary screening assay (not shown) Aβ steady state was assessed in two independent screening following drug treatments at 100 μM; Aβ-lowering drugs ≧15% compared relative to vehicle treated cultures) were selected for a secondary dose responses screening (range 0.01-100 μM). The drug dose response curve was analysis using sigmoid dose-response (variable hillslope) non-linear fitting method (Prism software, GraphPad). The drug concentration that provokes a response halfway between baseline and maximum (EC50) was derived from equation: Y=Bottom+(Top-Bottom)/(1+10̂((LogEC50-X)*hillslop)). The X value is logarithm of drug concentration; The Y value is Aβ level; Top is the highest Aβb level measured; bottom is the lowest Aβb level measured. Among the agents screened in this secondary dose-response assessment, 24 exerted Aβ-lowering activities at low, physiologically relevant concentrations reflected by EC50 for Aβ₁₋₄₀ and/or Aβ₁₋₄₂ reduction (shown in Table 1)

| | | | | | LDH at | |
|---|---|---|---|---|---|---|
| | | | EC50 (molar) | | E-5M | Ratio |
| | Drug Name | Pharmacological activities | Aβ1-40 | Aβ1-42 | % of change | Aβ1-40:Aβ1-42 |
| | α NE-blocker | | | | | |
| 1 | PRAZOSIN HYDROCHLORIDE | anti-hypertensive | 5.71E−04 | 6.76E−05 | 5.0% | 8.45 |
| 2 | YOHIMBINE HYDOCHLORIDE | blood pressure control (used in | no fit | 7.08E−12 | 6.0% | — |
| | | erectile dysfunction) | | | | |
| | β NE-blocker | | | | | |
| 3 | PROPRANOLOL HYDROCHLORIDE (+/ | anti-hypertensive | 5.75E−05 | 4.68E−05 | 6.0% | 1.23 |
| 4 | LABETALOL HYDROCHLORIDE | anti-hypertensive | 4.06E−09 | 7.19E−07 | −5.0% | 0.01 |
| | α,β NE-blocker | | | | | |
| 5 | CARVEDILOL | anti-hypertensive | 6.61E−05 | 1.32E−04 | | 0.50 |
| | Calcium-blocker | | | | | |
| 6 | FEDLINE HYDROCHLORIDE | vasodilation | 1.25E−05 | 4.20E−05 | −3.0% | 0.30 |
| 7 | NITRENDIPINE | anti-hypertensive | 8.22E−04 | 1.60E−06 | 5.0% | 514.04 |
| 8 | NIFEDIPINE | anti-hypertensive | no fit | 7.36E−08 | 12.0% | — |
| 9 | NIMODIPINE | anti-hypertensive (used in cerebral | no fit | 2.81E−05 | −4.0% | — |
| | | vasospasm; vascular dementia) | | | | |
| | Angiotensin Receptor Blocker | | | | | |
| 10 | VALSARTAN | anti-hypertensive | 1.44E−05 | 1.79E−05 | 13.0% | 0.80 |
| | Angiotensin-Converting-Enzyme Inhibitor | | | | | |
| 11 | PERINOPRIL ERBUMINE | anti-hypertensive | no fit | 4.05E−09 | −5.6% | — |
| | Vasodilation | | | | | |
| 12 | SULOCTIDIL | anti-hypertensive (not used, toxic) | 1.45E−05 | 2.13E−05 | −3.4% | 0.68 |
| 13 | RESERPINE | anti-hypertensive (not used, toxic) | 5.66E−05 | 9.02E−04 | −3.4% | 0.06 |
| 14 | HYDRALAZINE HYDROCHLORIDE | anti-hypertensive (not used, toxic) | 9.29E−06 | 1.04E−05 | −1.2% | 0.90 |
| 15 | ISOXSUPRINE HYDROCHLORIDE | anti-hypertensive (used in premature | no fit | 8.07E−06 | 5.0% | — |
| | | labour) | | | | |
| 16 | PHENTOLAMINE HYDROCHLORIDE | vasodilation (used in erectile dysfunction) | 1.21E−05 | 8.65E−08 | −3.4% | 140.28 |
| 17 | PAPARVERINE HYDROCHLORIDE | vasodilation (used in erectile dysfunction) | 1.72E−05 | 1.91E−05 | −8.9% | 0.90 |
| 18 | PROTOVERATRINE B | anti-hypertensive (not used) | 6.73E−06 | no fit | −2.5% | — |
| | Diuretics | | | | | |
| 19 | ETHACRYNIC ACID | anit-hypertensive | 2.10E−05 | 2.25E−05 | 10.0% | 0.93 |
| 20 | BEDROFUMETHIAZIDE | anit-hypertensive | 3.61E−05 | 1.37E−01 | 5.0% | 0.0003 |
| 21 | AMILORIDE HYDROCHLORIDE | anit-hypertensive | 8.67E−05 | 3.90E−02 | 6.4% | 0.0022 |
| | Anti-arrhythmic | | | | | |
| 22 | AMIODARONE HYDROCHLORIDE | cardiac arrhythmia | 1.53E−05 | 4.06E−05 | 1.9% | 0.38 |
| 23 | DISOPYRAMIDE PHOSPHATE | ventricular arrhythmias | 7.78E−10 | 5.33E−05 | −3.7% | 0.00001 |
| | Anti-coagulant | | | | | |
| 24 | DIPYRIDAMOLE | secondary stroke prevention | 1.66E−05 | 1.24E−06 | 6.4% | 13.37 |

Following the identification of 21 candidate commercially available “cardiovascular antihypertensive drugs” with Aβ-lowering activity, various candidate drugs were selected for further pre-clinical investigation in mouse models of AD. Based on this consideration, an extensive literature search was performed on each of the agents in peer-reviewed journal articles published between 2003 and 2006 MEDLINE database search encompassing 1) clinical pharmacology, 2) indications and usage, 3) contraindications/precautions, 4) the availability of information about dosages and administration, and 5) safety information supporting the possibility of immediate application in the geriatric population. Based on this criteria, and the evidence of preferential Aβ₁₋₄₂ lowering activities (Table I), an initial 9 candidates drugs were selected for further preclinical investigation, all of which are currently prescribed antihypertensives (Table II). Thus, based on clinically available information, as well as Aβ-lowering activities as determined by in vitro screening studies, studies WERE initiated to confirm the clinical relevance of the hypothesis that antihypertensives may prevent or attenuate AD-type neuropathology and cognitive deterioration in the Tg2576 mouse models of AD.

TABLE II
A list of nine most effective Aβ-lowering currently prescribed antihypertensive drugs.
Physiological activities, EC50 for Aβ₁₋₄₀ and Aβ₁₋₄₂ peptides,
the range of clinical doses and the calculated mouse equivalent doses corresponding
the human doses. See text for more information about calculations of mouse equivalent
doses.

| | | | | | | | | Mouse | |
|---|---|---|---|---|---|---|---|---|---|
| | | Pharmacological | | EC50 (molar) | | Clinical | | Equivalent | |
| | Drug name | Activities | Categories | Aβ1-40 | Aβ1-42 | Dose | | Dose | |
| 1 | LABETALOL | anti-hypertensive | β NE-blocker | 4.06E−09 | 7.19E−07 | 200-400 | mg/day; p.o. | 36.9-73.9 | mg/kg |
| | HYDROCHLORIDE | | | | | | | | |
| 2 | PERINDOPRIL ERBUMINE | anti-hypertensive | ACE inhibitor | no fit | 4.50E−09 | 4-16 | mg/day; p.o. | 0.7-3.0 | mg/kg |
| 3 | NIFEDIPINE | anti-hypertensive | Calcium blocker | no fit | 7.36E−08 | 30-120 | mg/day; p.o. | 5.5-22.2 | mg/kg |
| 4 | NITRENDIPINE | anti-hypertensive | Calcium blocker | 8.22E−04 | 1.60E−06 | 20-40 | mg/day; p.o. | 3.7-7.4 | mg/kg |
| 5 | VALSARTAN | anti-hypertensive | Angiotensin | 1.44E−05 | 1.79E−05 | 80-320 | mg/day; p.o. | 14.8-59.1 | mg/kg |
| | | | Receptor Blocker | | | | | | |
| 6 | NIMODIPINE | anti-hypertensive | Calcium blocker | no fit | 2.81E−05 | 90-180 | mg/dy; p.o. | 16.6-33.2 | mg/kg |
| | | (used in cerebral | | | | | | | |
| | | vasospasm; vascular | | | | | | | |
| | | dementia) | | | | | | | |
| 7 | PROPRANOLOL | anti-hypertensive | β NE-blocker | 5.75E−05 | 4.68E−05 | 80-640 | mg/day; p.o. | 14.8-118.2 | mg/kg |
| | HYDROCHLORIDE | | | | | | | | |
| 8 | PRAZOSIN HYDROCHLORIDE | anti-hypertensive | α NE-blocker | 5.71E−04 | 6.76E−05 | 1-20 | mg/dy; p.o. | 0.2-3.7 | mg/kg |
| 9 | CARVEDILOL | anti-hypertensive | α,β NE-blocker | 6.61E−05 | 1.32E−04 | 12.5-50 | mg/dy; p.o. | 2.3-9.2 | mg/kg |

### Preclinical Characterization of Valsartan as a Potential Novel Agent in the Prevention of AD

Among the agents listed in Table II, it was decided to prioritize the angiotensin receptor type-1 (AT-1) blocker, valsartan as the first candidate for AD therapeutic development. Valsartan is the S-enantiomer of N-(1-oxopentyl)-N-[[2-(1H-tetrazol-5-yl) [1,1-biphenyl]-4-yl]methyl]-L-valine; the presence of an acylated amino acid residue in valsartan contributes to its high binding affinity to the AT-1 receptor and prolonged receptor occupancy (Thomas and Johnston, 2004). Valsartan was chosen primarily on the considerations that, 1) it is an effective Aβ₁₋₄₂ lowering agent (Table II ) and is therefore highly relevant for potential future clinical application in AD, 2) it is widely prescribed and one of the most safe antihypertensive agents in the geriatric population (Ogihara et al, 2004; Ripley 2005; Unger et al, 2003), 3) it has minor hypotensive effects in normotensive conditions (Yamamoto et al., 1997), and 4) it blocks AT-1 receptors whose expression is elevated in the brain of AD (Savaskan et al, 2001). The valsartan studies were conducted using Tg2576 mice, a well-established mouse model of AD-type amyloid neuropathology and cognitive deterioration (Hsiao et al., 1996; Wang et al., 2005; Ho et al., 2004;). The Tg2576 mouse model of AD was primarily considered for the proposed studies due to 1) its widespread use in the characterization of AD modifying agents for AD therapeutic development (Conte et al., 2004; Lee et al., 2004) and 2) our extensive experience in the preclinical characterization of this mouse model in the assessment of AD modifying strategies (e.g. dietary restriction) (Wang et al., 2005; Appendix 3).

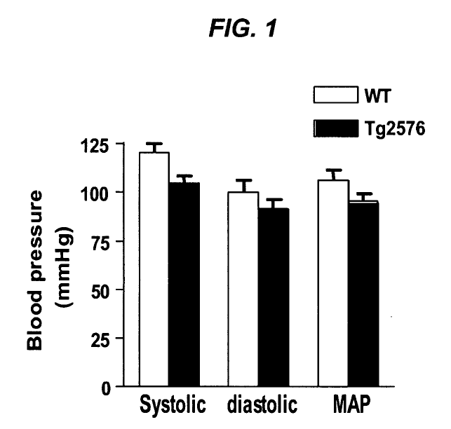

Because the goal of the proposed application is the preclinical characterization of antihypertensive drugs with Aβ-lowering activities, it was decided to proceed with studies assessing baseline blood pressure profiles in the Tg2576 mouse model. Consistent with previous evidence (Zhang et al, 1997), it was found that systolic, diastolic, and mean arteriole blood pressure in ˜11 month old female mice did not significantly differ relative to that in strain-, age-, and gender-matched wild-type mice (FIG. 1). Based on this evidence the role of valsartan treatment in the attenuation of cognitive deterioration and eventually the prevention of AD-type amyloid neuropathology in the Tg2576 strain was assessed.

### Chronic Valsartan Treatment is Highly Tolerable in Normotensive Tg2576 Mice

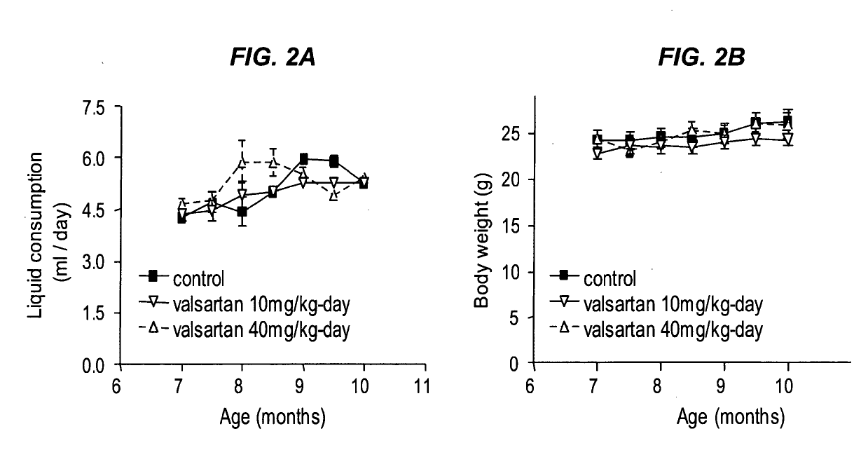

In assessing tolerability, valsartan was delivered to mice in their drinking water at doses comparable to those prescribed in the clinical setting for hypertension (online Physicians' Desk Reference). If preclinical efficacy studies showed that valsartan could prevent AD-type cognitive deterioration or neuropathology at clinically relevant doses, this information could be readily translated into a potential AD therapeutic application. In calculating equivalent doses of valsartan to be delivered to Tg2576, FDA-recommended criteria were applied, which takes into consideration body surface area (FDA; 2005). The clinically recommended dose of valsartan in humans (80-320 mg/day ) was caculated (online Physicians' Desk Reference, which also may be referred to for the concentrations of the other antihyerptensives to be used) corresponds to 15-60 mg/kg per day in mice [human equivalent dose in mg/kg=animal dose in mg/kg×(animal weight in kg/human weight in kg)0.33]. Based on this consideration, Tg2576 mice were treated with 10 or 40 mg/kg-day valsartan provided in drinking water ad libitum, starting at ˜7 months of age, prior to the development of AD-type neuropathology and cognitive deterioration (Hsiao et al., 1996). In a parallel control study, age and gender-matched Tg2576 mice were provided with regular drinking water. Valsartan treatments continued for ˜4 months and mice were assessed for cognitive dysfunction (and eventually AD-type neuropathology) at ˜11 months of age (an age at which female Tg2576 normally develop significant AD-type amyloid plaque neuropathology and Aβ-related cognitive impairment) (Hsiao et al., 1996). Treatment with valsartan 10 or 40 mg/kg-day in Tg2576 mice did not significantly influence the amount of fluid intake throughout the entire exposure period, relative to the group having received normal drinking (FIG. 2A). Based on this evidence, and the fact that our ·7-11 month old mice (˜20-25 g in weight) drank ˜4-5 ml/day of water containing 50 mg/L or 200mg/L of valsartan salt respectively, it resulted that the two treatment groups received ˜10 or 40 mg/kg-day valsartan, respectively. The evidence that valsartan treatment did not influence amount of water intake is highly relevant and suggests that appropriate doses of valsartan were accurately delivered across the entire treatment period. Consistent with this evidence, no detectable changes in body weight in response to valsartan treatment were found (FIG. 2B). Collectively, this evidence confirms the high tolerability of valsartan in the setting of chronic administration and provides support for the use of this agent in the treatment of the AD phenotype.

Treatment with Valsartan Beneficially Attenuates Spatial Memory Deterioration as Assessed by Morris-maze (MWM) Test in Tg2576 Mice

Based on the evidence that valsartan is a highly safe antihypertensive drug in humans, as well as our finding that chronic valsartan treatment is highly tolerable in Tg2576 (FIG. 2A,B) and WT control mice (data not shown), the influence of valsartan on AD was assessed. AD-type cognitive deterioration was assessed by the classical MWM, a routine modality in our laboratory (Ho et al., 2004) and a commonly used means of assessing spatial memory function in mouse models of AD (see Research Design for more information; Ho et al., 2004). In the MWM assay, experimental animals are placed into a circular water tank and provided with a submerged “escape platform” at a specific location. Appropriate visual cues are located on a wall surrounding the water tank. Through repeated “learning trials”, normal animals typically learn to use the visual cues for spatial navigation and eventually require less time to swim to the platform as reflected by reducing escape latency.

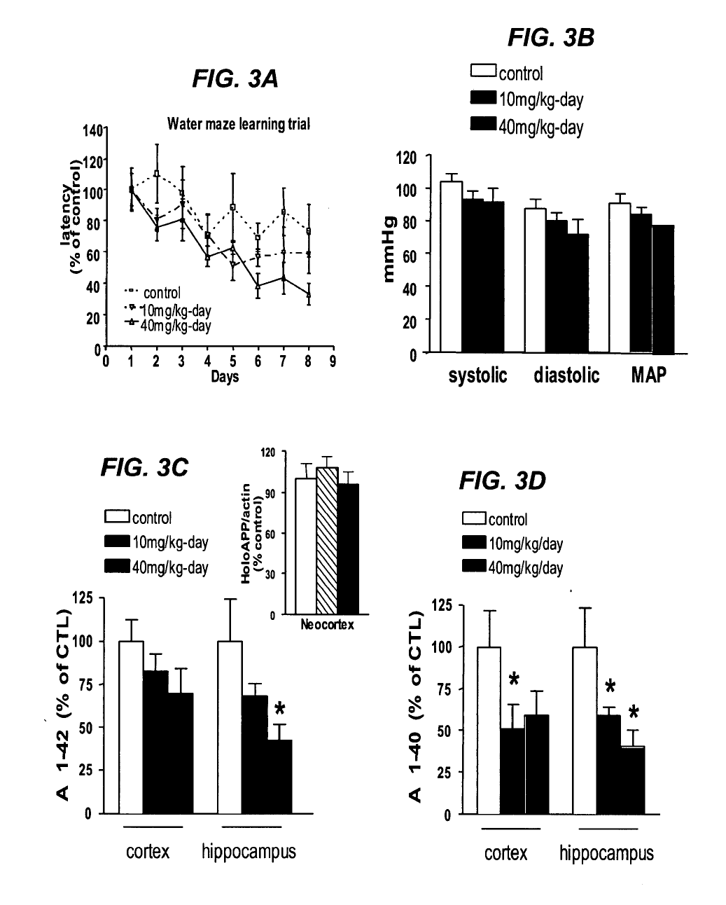

It was found that ˜11 month old untreated Tg2576 (control) mice exposed to regular drinking water failed to learn how to use the visual cues as reflected by no improvement in the escape latency over increasing learning trials (FIG. 3A). This finding is indicative of spatial memory impairment in mice of this age, as previously reported (Hsiao et al., 1996; Ho et al., 2004) (FIG. 3A). Additionally, it was found that treatment of Tg2576 mice with valsartan for ˜4 months resulted in a significant improvement in escape latency during the learning trials (FIG. 3A). Most importantly, this improvement in escape latency was dose-dependent, reflected by the fact that the Tg2576 mouse group receiving 40 mg/kg/day performed significantly better than the group receiving 10 mg/kg/day (two-way ANOVA; 10 mg/kg/day valsartan treated group vs regular water-drinking group, F1,7793=4.913, p<0.00288 for drug treatment, F7,23660=2.131, p<0.0466 for escape latency over time; 40 mg/kg/day valsartan treated group vs regular water-drinking group, F1,19840=14.28, p<0.0003 for drug treatment, F7,34510=3.549, p<0.0018 for escape latency over time (FIG. 3A). In parallel control studies, contrary to what was found in Tg2576 mice, it was found that valsartan treatment at either 10 and 40-mg/kg/day treatment in strain-, age-, and gender-matched WT mice did not significantly influence spatial memory performance assessed by MWM compared with untreated WT mice.

In view of the antihypertensive properties of valsartan, control studies continued to monitor the blood pressure in the same mice used for behavioral assessments (prior to them being sacrificed for neuropathological studies described below). Interestingly, ˜4 month treatment with either 10 or 40 mg/kg-day of valsartan in normotensive Tg2576 mice did not significantly influence systolic, diastolic, or MAP (FIG. 3B), relative to control Tg2576 mice receiving regular drinking water. Similarly, no detectable changes in systolic, diastolic, or MAP were found in age-, gender-, and strain-matched WT-valsartan treated mice, relative to the control WT mice (data not shown). This evidence is consistent with previous studies showing that chronic valsartan treatment in normotensive rats does not influence blood pressure profiles (Yamamoto et al, 1997) and provides a basis for interpreting the studies proposed in the present invention.

Valsartan Attenuates Spatial Memory Function Deterioration in Tg2576 Mice Coincidental with a Reduction in Aβ1-40 and Aβ1-42 in the Brain

Based on the preliminary evidence suggesting that valsartan may beneficially influence AD-type cognitive deterioration in Tg2576 mice, the relationship of this finding to AD-type amyloid neuropathology in the brain of the same mouse groups used for behavioral studies was assessed. In preliminary studies it was determined that valsartan treatment in Tg2576 mice coincided with reduction of amyloidogenic Aβ1-42 in the hippocampal formation (40/mg-kg, p<0.05) or Aβ1-40 in the hippocampal formation (10 mg/kg, p<0.05; 40 mg/kg, p<0.01) and neocortex (40 mg/kg, P<0.01; post-hoc, one way ANOVA) as assessed by ELISA assay, relative to untreated age and gender-matched Tg2576 control mice (FIG. 3C, D). Moreover, no detectable change in total full-length (holo)APP levels were noted in the brain of valsartan treated relative to untreated controls (FIG. 3C, inset), thus excluding the possibility that the Aβ-lowering activity of valsartan in vivo is due to decreased APP transgene expression. Ongoing neuropathology studies assessing amyloid plaque pathology in valsartan-treated and control (untreated) Tg2576 mice are currently in progress in our laboratory.

A series high throughput studies were initiated to assess and quantify antihypertensive drug levels and related metabolites in Tg 2576 and WT controls following in vivo treatments. Excitingly, in preliminary/feasibility studies using Biosystems QTRAP mass spectrometery enabling high-sensitive multiple reaction monitoring (MRM) have successfully detected steady state levels of valsartan concentrations in the blood (serum) of chronically valsartan-treated mice (40 mg/kg). Presently, using QSTAR (QqTOF) mass spectrometry, which delivers high sensitivity and resolution as well as unparalleled mass accuracy for determination of molecular weights of drugs and related metabolites at femtomole levels, the inventor is proceeding with studies aimed at detecting and eventually quantitating antihypertensive drugs and related metabolites in the brain or CSF. Thus, these technologies will accurate determination of the penetration of the blood brain barrier of candidate Aβ lowering antihypertensives in response to chronic treatments in vivo.

Collectively, these studies strongly support the possibility that valsartan is a safe Aβ-lowering antihypertensive agent, and can be further exploited for use in attenuating (or possibly preventing) AD-cognitive deterioration through mechanisms independent of its antihypertensive activity. Moreover, in view of our preliminary evidence that valsartan treatment in age-, gender-, and strain-matched WT mice did not influence spatial memory functions at any dose examined, our studies strongly support the hypothesis that valsartan may beneficially influence cognitive deterioration in Tg2576 by modulating Aβ-amyloid neuropathology. This evidence strongly supports the use of antihypertensive agents as potential preventative/therapeutic agents in AD.

### Microvascaulture in the Brain Tg2576 Mice as Additional Outcome Measures for the Beneficial Role Aβ Lowering Antihypertensive Drugs

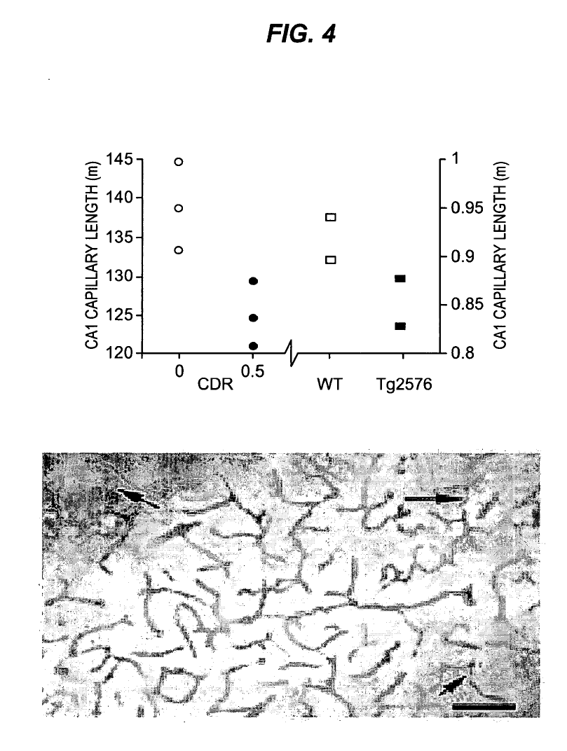

There is ample evidence that of microvasculature pathology in the AD brain, including thinning of microvessels referred to as atrophic or string vessels (Hassler, 1965) and fragmentation of the microvasculatrure related to a decreased in the number of long microvessels and their branches (Buee et al, 1994). It is likely these abnormal microvascular alterations may increase the risk of AD dementia. Limited evidence suggested that anomalies in microvascular structure and functions might be also presented in the brain of the Tg2576 mouse model of AD which may contribute to age-related cognitive deterioration. In particular, Christie, et al. (2001) showed that the structure and function of smooth muscle cells in the walls of pial vessels are aversely affected by amyloid deposition in the brain of Tg2576 mice. Based on this consideration, it was hypothesized that treatments which reduces Aβ neuropathology in the brain of Tg2576 mice may also result in attenuation (or reversal) of vascular pathology. A series of studies have been initiated to characterize in detail the disrupted microvasculature morphological feature in the brain of Tg2576 mice and the impact of reducing Aβ neuropathology in response to Aβ lowering antihypertensive drug treatements (e.g. valsartan). Based on this consideration, the characterization of microvasculature morphological feature in the brain of Tg2576 mice was done and compared with those in the AD brain. In feasibility preliminary studies, it was found evidence of reduced capillary length (FIG. 4, top panel) and collapsed/fragmented vessels (not shown) in the brain (hippocampal formation) of early AD cases. Excitingly, similar reduction was observed in length of capillaries (FIG. 4, top panel) as well as (qualitatively) increased presence of collapsed and fragmented vessels (FIG. 4, bottom panel) are also presented in the hippocampal formation of ˜11-month old Tg2576 mice. These encouraging preliminary data are consistent with the hypothesis that the Tg2576 mice present AD-type microvasculature abnormalities and supports investigation and development of Aβ-lowering antihypertensives (e.g. valsartan)-based treatments to attenuate (or reverse) AD-type microvasculature abnormalities. This information will provide additional supportive evidence for consideration of selective Aβ-lowering antihypertensives for the therapeutic applications described herein.

### Molecular Topological Analysis of Cardiovascular Antyhypertensive Drugs to Identify Predicative Criteria of Aβ-Lowering Activities

Based on the encouraging results from our high-throughput cardiovascular drug screening (discussed above), it was hypothesized that Aβ-lowering cardiovascular drugs may share common topological characteristics that could be exploited in the future development of Aβ-lowering AD therapies. To test this hypothesis, a series of studies were initiated aimed at identifying such selective molecular structural characteristics among the candidate cardiovascular agents.

Traditionally, molecular structural analyses are based primarily on electron densities, surface characteristics, and similar “physical” attributes. However, recent development of analytic methods utilizing molecular topological indices (distinct from simple structural analyses) has proven more effective in predicting useful chemical structures and scaffolds than standard approaches (Galvez et al, 1995; Galvez et al, 1996; Galvez, et al, 2001; Jesus et al, 1999). The topological abstractions of the structures and associated biological information (needing only to be based on 2 dimensional representations of the structures) used in our studies were derived from a set of ˜1,000 indices created by Dr. Jorge Galvez (University of Valencia, Spain). Molecular topological indices have been used successfully in identifying analgesic compounds (Galvez et al, 1994), cytostatic agents (Galvez et al, 1996), antibacterial agents (Rafael et al, 2000), antihistamines and novel, specific tyrosine kinase inhibitors (Ingolia, Personal Communication). Based on this consideration, a program utilizing specific mathematical molecular descriptors (molecular topological indices) to identify features predictive of Aβ-lowering activity among the candidate cardiovascular agents has been initiated.

In preliminary studies, it was found a degree of fit of 0.9 for a model created with select molecular topological indices and Aβ-lowering activity in the complete set of 150 commercially available cardiovascular drugs used in our high-throughput drug studies. This finding confirms that the dataset is suitable for identifying useful predictions. Based on this evidence, the studies were designed to further optimize the selection process of specific molecular topological indices with improved predictability for Aβ-lowering activity. This information will be used for the further preclinical development of novel, Aβ-lowering compounds for AD prevention and/or therapeutics.

There is no information on how hypertensive drugs may specifically modulate Aβ contents in vitro or in vivo. Antihypertensive activities of valsartan, perindopril erbumine, amiloride hydrochloride and prazosin hydrochloride and carvedilol are attributed to, respectively angiotensin receptor AT1 inhibition, angiotensin-converting enzyme inhibition, diuretic, a-adrenergic blocker and a,b-adrenergic blocker activities. However, it is unlikely these physiological properties are directly involved in mediating Aβ-lowering activities. For example, our priority list of commonly prescribed antihypertensive drugs for preclinical characterization that are selected based on their Aβ-lowering activities represent multiple clinical indications: angiotensin receptor blocker (valsartan), angiotensin-converting enzyme inhibitor (perindopril erbumine), diuretic (amiloride hydrochloride), a adrenergic blocker (prazosin hydrochloride) and α,β adrenergic blocker (carvedilol). However, many other effective antihypertensive drugs characterized by comparable clinical indications do not exert Aβ-lowering activities. Valsartan, perindopril erbumine, amiloride hydrochloride, prazosin hydrochloride and carvedilol may reduce Aβ contents by yet characterized activities, most likely unrelated to their antihypertensive efficacy, which ultimately may interfere with Aβ generation from the amyloid precursor protein or may promote Aβ degradation. Aβ peptides are generated by sequential cleavage of the amyloid precursor protein by β- and γ-secretase (Xia, 2001; McLendon et al., 2000; Vassar and Cintron, 2000). In contrast, α-secretase cleaves within the Ab peptide sequence and precludes the formation of Aβ peptides (McLendon et al., 2000).

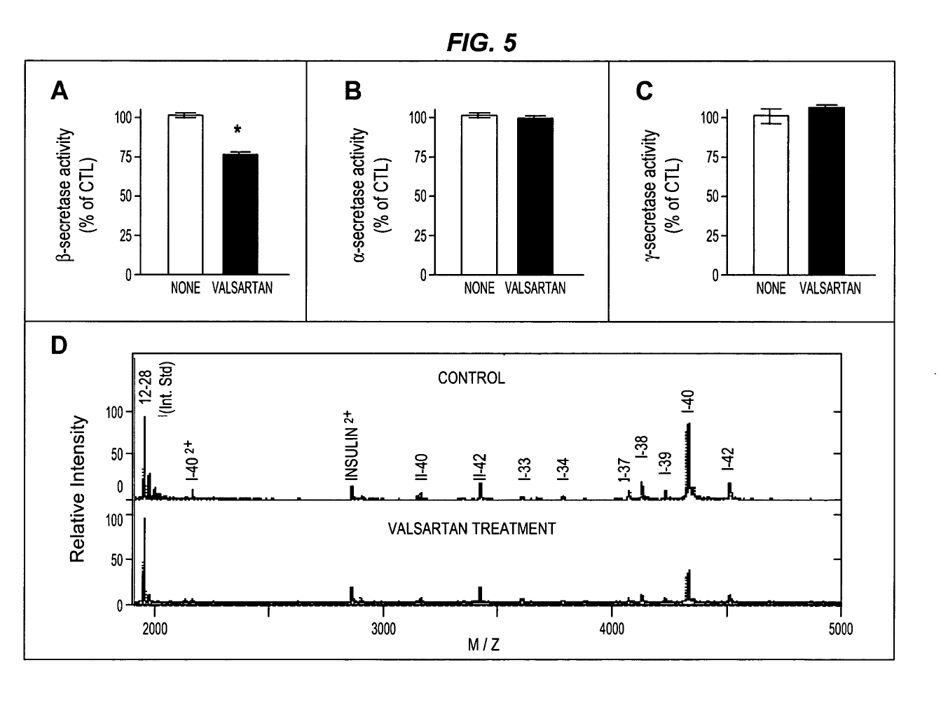

In preliminary in vitro studies to assess the potential impact of antihypertensive drugs on the generation of Aβ peptides, it was found the anti-amyloidogenic activity of valsartan is coincidental with selectively inhibition of β-secretase activity in primary Tg2576 neuron cultures (FIG. 5A); no detectable change in α- and γ-secretase activities in response to valsartan was observed in the same neuron cultures (FIG. 5B, 5C).

The above described studies show that antihypertensive agents have Aβ-lowering properties and will be useful for AD prevention and/or treatment.

### Example 2

### Further Investigations

Based on the results shown in Example 1 and the fact that fact that Aβ neuropathology is a major hallmark in the AD brain and a major target for pharmacological intervention, a high throughput drug screening of 55 of the most commonly prescribed antihypertensive drugs aimed at identifying Aβ-lowering activity (Table III). From this high-throughput dose-response screening studies (Table IV), the inventors found that 7 out of the 55 antihypertensive drugs examined were capable of significantly reducing Aβ1-42 and/or Aβ1-40 steady state levels in the conditioned medium of primary cortico-hippocampal neuron cultures generated from Tg2576 embryos (E14), relative to parallel vehicle-treated control primary neuron cultures. Most importantly, we found that each of the 7 drugs exerted dose-dependent Aβ-lowering activity with a predicted drug concentration resulting in 50% Aβ1-42 and/or Aβ1-40 inhibition (EC50) at low μM range (Table IV). No apparent neurotoxicity was associated with any of the agents, as assessed by a lactate dehydrogenase activity assay in parallel cultures at identical drug concentrations (Table IV). As shown in Table IV, the 7 antihypertensives belong to 6 separate subclasses: 1) β-adrenergic blockers, propranolol-HCL; 2) α/β-adrenergic blockers, carvedilol; 3-4) angiotensin-II type-I receptor blockers (ARBs), losartan and valsartan; 5) Ca++channel receptor blockers, nicardipine-HCL; 6) K+-sparing diuretics, amiloride and 7) vasoldilators, hydralazine.

The inventors assessed for the potential Aβ-lowering activity of 55 drugs clinically prescribed for hypertension, using primary cortico-hippocampal neuron cultures derived from embryonic (E16) Tg2576 AD mice, as previously described in the lab (Wang et al., 2005; Zhao et al., 2005). Neuron cultures were maintained in a serum-free Neurobasal medium in the presence of L-glutamine and B27 supplement as is standard condition in our lab, described in Mirjany et al. (2002). For in vitro treatments drugs available from “The Spectrum Collection” in stocks of 10 mM in DMSO were applied directly to cultures to the desired concentrations (final 1% DMSO); control primary neuron cultures from Tg2576 embryos were treated with vehicle resulting in a final 1% DMSO. Conditioned mediums were collected 24 hr after treatment for Aβ1-42 and Aβ1-40 content, assessed by quantitative Aβ ELISA assays, as previously discussed (Ho et al., 2004; Wang et al., 2005; Wang et al., 2007). In a primary screening (not shown), Aβ steady state levels were assessed in two-independent assays following drug treatments at 100 μM. Drugs which reduced Aβ content in the conditioned medium by >15%, relative to vehicle-treated cultures, were selected for a secondary dose-response screening study with a drug treatment ranging from 0.01-100 μM. The drug dose-response curve was analyzed using a sigmoid dose-response (variable hillslope) non-linear fitting method (Prism software, GraphPad). The drug concentration that provoked a response halfway between baseline and maximum (EC50) was derived from equation: Y=Bottom+(Top-Bottom)/(1+10̂((LogEC50-X)*hillslop)), where the X value is logarithm of drug concentration; The Y value is Aβ level; top is the highest Aβ level measured; bottom is the lowest. None of the 7 drugs exerted cytotoxicity at 50 μM drug concentration, as evaluated by LDH assay.

TABLE III
α-ADRENERGIC BLOKERS

| | |
|---|---|
| 1 | PRAZOSIN HYDROCHLORIDE |
| 2 | URAPIDIL |

β-ADRENERGIC BLOCKERS

| | |
|---|---|
| 3 | PROPRANOLOL HYDROCHLORIDE |
| 4 | LABETALOL HYDROCHLORIDE |
| 5 | ATENOLOL |
| 6 | METOPROLOL TARTRATE |
| 7 | ALPRENOLOL |
| 8 | NADOLOL |
| 9 | PINDOLOL |
| 10 | PRACTOLOL |
| 11 | TIMOLOL MALEATE |
| 12 | ACEBUTOLOL HYDROCHLORIDE |

α/β-ADRENERGIC BLOCKERS

| | |
|---|---|
| 13 | CARVEDILOL |

ANGIOTENSIN CONVERTING ENZYME INHIBITOR

| | |
|---|---|
| 14 | PERINDOPRIL ERBUMINE |
| 15 | FOSINOPRIL SODIUM |
| 16 | ENALAPRIL MALEATE |
| 17 | CAPTOPRIL |
| 18 | RAMIPRIL |
| 19 | BENAZEPRIL HYDROCHLORIDE |
| 20 | QUINAPRIL HYDROCHLORIDE |
| 21 | TRANDOLAPRIL |

ANGIOTENSIN RECEPTOR BLOCKERS

| | |
|---|---|
| 22 | VALSARTAN |
| 23 | LOSARTAN |
| 24 | OLMESARTAN MEDOXOMIL |
| 25 | TELMISARTAN |
| 26 | IRBESARTAN |
| 27 | CANDESARTAN CILEXTIL |

CALCIUM-CHANNEL BLOCKERS
Dihydropyridine

| | |
|---|---|
| 28 | NICARDIPINE HYDROCHLORIDE |
| 29 | NITRENDIPINE |
| 30 | FLUNARIZINE HYDROCHLORIDE |
| 31 | NIFEDIPINE |
| 32 | NIMODIPINE |
| 33 | AMLODIPINE BESYLATE |

Non-dihydropyridine

| | |
|---|---|
| 34 | DILTIAZEM HYDROCHLORIDE |
| 35 | VERAPAMIL |

DIURETICS
Thiazide diuretics

| | |
|---|---|
| 36 | BENDROFUMETHIAZIDE |
| 37 | HYDROFLUMETHIAZIDE |
| 38 | HYDROCHLOROTHIAZIDE |
| 39 | METOLAZONE |
| 40 | INDAPAMIDE |
| 41 | CHLOROTHIAZIDE |
| 42 | CHLORTHALIDONE |

Loop diuretics

| | |
|---|---|
| 43 | BUMETANIDE |
| 44 | ETHACRYNIC ACID |
| 45 | TORSEMIDE |
| 46 | FUROSEMIDE |

Potassium-sparing diuretics

| | |
|---|---|
| 47 | AMILORIDE HYDROCHLORIDE |
| 48 | TRIAMTERENE |

Aldosterone antagonists

| | |
|---|---|
| 49 | SPIRONOLACTONE |

OTHER MECHANISMS OF ACTION
Direct vasodilators

| | |
|---|---|
| 50 | HYDRALAZINE HYDROCHLORIDE |
| 51 | MINOXIDIL |
| 52 | DIAZOXIDE |
| 53 | ISOXSUPRINE HYDROCHLORIDE |

Centrally active agents

| | |
|---|---|
| 54 | METHYLDOPA |

Ganglion blockers

| | |
|---|---|
| 55 | GUANETHIDINE SULFATE |

TABLE IV

| | EC50 (M): Ab-lowering activity | | |
|---|---|---|---|
| | | | LDH |
| | Abeta1-40 | Abeta1-41 | (at 5F-5 M) |
| beta-adrenergic | | | |
| blockers | | | |
| PropranololHCL (+/−) | 5.75E−5 | 4.68E−5 | no change |
| alpha/beta-adrenergic | | | |
| blockers | | | |
| Carvedilol | 6.61E−05 | 1.32E−4 | no change |
| angiotensin-receptor | | | |
| blockers | | | |
| Valsartan | 1.44E−5 | 1.79E−5 | no change |
| Losartan | 1.17E−4 | 1.00E−4 | no change |
| calcium-blockers | | | |
| NicardipineHCl | 3.64E−5 | 1.19E−4 | no change |
| diuretics | | | |
| amiloride | 8.67E−5 | 3.01E−5 | no change |
| vasodilators | | | |
| HydralazineHCl | 9.29E−6 | 1.04E−5 | no change |

Thus, the in vitro high throughput drug studies strongly support the hypothesis that certain drugs commonly prescribed for hypertension may influence mechanisms associated with generation and or clearance of Aβ peptides in vitro. Interestingly, our studies suggest that the Aβ-lowering activity was limited to a specific set of antihypertensive drugs. Based on this encouraging evidence, a series of dose optimization studies were initiated in vivo in Tg2576 mice for the all the candidate antihypertensive Aβ-lowering drugs identified.

Development of a Short-Term Drug Dosing Treatments in the Optimization of Drug Selection and Identification of Efficacious Dosage for Long-Term Studies.

Because the overall goal of the studies proposed in this application is the preclinical characterization of antihypertensive-Aβ-lowering activities in Tg2576 mice, the inventors decided first to proceed with control studies to assess baseline blood pressure profiles in the Tg2576 mouse model. Consistent with previous evidence (Zhang et al., 1997), it was found that systolic, diastolic, and MAP in ˜6-7 month old Tg2576 mice did not significantly differ relative to that in strain-, age-, and gender-matched wild-type mice to Tg2576 (FIG. 1). Based on this evidence, the inventors initiated a series of short term dosing studies to explore efficacious doses able to attenuate Aβ neuropathology in young-adult mice at doses below or within those recommended for the treatment of hypertension. In calculating actual doses of candidate antihypertensive drugs, we applied an FDA-recommended criteria which takes into consideration body surface area across species, among other factors (see Table V).

TABLE V

| | recommended | | | | |
|---|---|---|---|---|---|
| | clinical doses | Tg2576 treatment | | Equivalent human dose | |
| | for hypertension | Clinical | Sub-clinical | Clinical | Sub-clinical |
| | treatment in human | dose | dose | dose | dose |
| | (mg/day) | (mg/kg/day) | (mg/kg/day) | (mg/day) | (mg/day) |
| Propranolol | 160-300 | 60 | 10 | 300 | 50 |
| Nicardipine | 50-100 | 18 | 3 | 100 | 17 |
| Losartan | 60-120 | 20 | 3 | 120 | 20 |

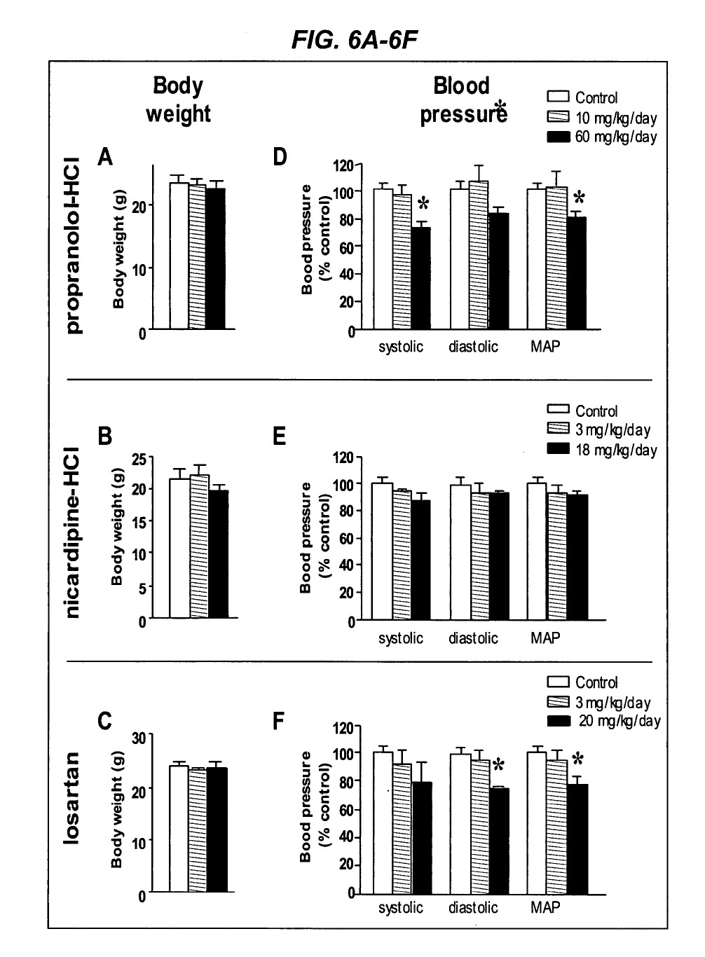

Surprisingly, these studies showed that short-term treatments of ˜6 months old young-adult Tg2576 mice with 10 or 60 mg/kg/day of propranol-HCL, or 3 or 18 mg/kg /day of nicardipine-HCL, or 3 or 20 mg/kg /day losartan (delivered in the drinking water) which are equivalent doses ˜3 fold below or within those recommended for the treatment for hypertension for these three drugs respectively, were well-tolerated in Tg2576 mice. Tolerability was determined by lack of significant change in body weight (FIG. 6A-C), as assessed 3 weeks post-treatment.

In this study ˜6 month old Tg2576 mice were exposed to propanolol-HCL, nicardipine-HCL, or losartan at doses below or within the range of that used for treatment of hypertension. FIG. 6A-C show body weight of mice in response to ˜3 weeks of treatment. FIG. 6A) propranolol-HCL; FIG. 6B) nicardpine HCL; FIG. 6C) losartan. FIG. 6D-F show the assessment of systolic, diastolic, and MAP blood pressure in Tg2576 mice in response to drug doses below or within the range of those prescribed in hypertension. Blood pressure measurements were conducted using a commercial blood pressure analysis system designed specifically for small rodents (Hatteras Instruments, NC). Following manufacturer's instruction, mice were temporarily immobilized in a restraining chamber and an inflated tail-cuff wrapped around the tail was used to measure pressures. Each blood pressure determination was calculated as the mean of 10 individual measurements per animal. In A-F, values represent mean±SEM values, n=3-5 mice per group. 2-tailed t-test: *P<0.05.

Finally the inventors also found that short-term dosing treatment of Tg2576 mice with propranolol-HCL (FIG. 6D), nicardipine-HCL (FIG. 6E), and losartan (FIG. 6F) at doses ˜3 fold below the minimal dose prescribed for the treatment of hypertension, did not significantly influence either systolic, diastolic or MAP blood pressure in Tg2576 mice, relative to untreated controls, three weeks after treatment.

However, the inventors also found that treatment of Tg2576 mice with propranolol-HCL (FIG. 6D), nicardipine-HCL (FIG. 6E) and losartan (FIG. 6F) at doses within the range of that used for treatment of hypertension resulted in decreased systolic, diastolic, and MAP blood pressure in Tg2576 mice, relative to control untreated Tg2576 mice, three weeks after treatment. No detectable changes in either systolic, diastolic, or MAP were found in response to treatment with nicardipine-HCL at any dose below or within the range of that prescribed for hypertension (3-18 mg/kg/day) (FIG. 6E). Based on this evidence, the inventors continued to explore the relationship of these changes with respect to the beneficial role of propranol-HCL, nicardipine-HCL and losartan in the attenuation of AD-type Aβ accumulation in the brain and plasma of the same mouse groups.

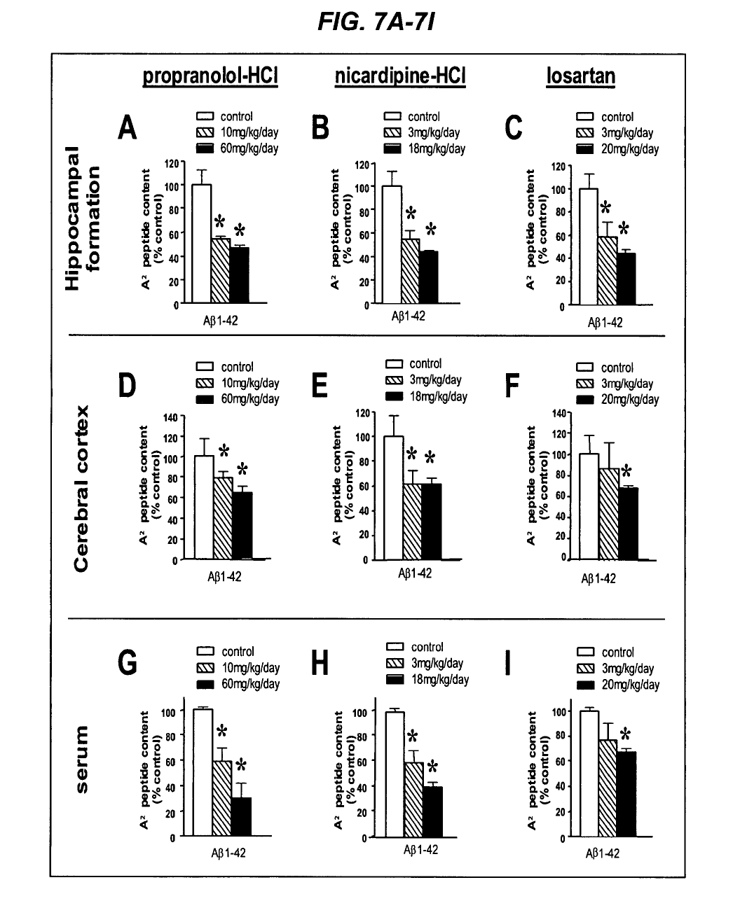

Consistent with the high-throughput drug screening in vitro, the inventors found that short-term dosing treatments with propranolol-HCL, nicardipine-HCL, or losartan, at either subclinical or clinical concentrations for the treatment of hypertension significantly reduced steady state levels of Aβ1-42 (and Aβ1-40, not shown) in the hippocampal formation (FIG. 7A-C) or cerebral cortex (piriform cortex) (FIG. 7D-F), as assessed by quantitative ELISA immunoassay. Collectively, our short-term dosing feasibility studies tentatively suggests that when propranolol-HCL, nicardipine-HCL, or losartan are delivered even at doses ˜3 fold below that recommended for treatment of hypertension, they can still exert significant Aβ-lowering activity in the brain of Tg2576 mice but, most importantly without influencing systolic, diastolic and MAP blood pressure (FIG. 6A-F).

In parallel studies exploring the regional distribution of Aβ-lowering activity, the inventors also found that propranolol-HCL and nicardipine-HCL, but not losartan, delivered in Tg2576 mice at doses ˜3 fold lower than the minimal recommended hypertension dose, prevented plasma accumulation of Aβ1-42 levels (and Aβ1-40) in the same mice used for brain studies, relative to untreated controls (FIG. 7G-I). Similarly, Aβ1-42 (and Aβ1-40)-lowering responses were found in Tg2576 mice in response to treatment with propranol-HCL, nicardipine-HCL, and losartan within the dosage range prescribed for hypertension. This evidence suggests that certain antihypertensive drugs may promote Aβ-lowering activity in the brain through either a central mechanism, or possibly through a “peripheral sinking mechanism,” both of which ultimately result in decreased Aβ content in the brain. The studies provide a basis for the use of these agents for a preventative and/or therapeutic role of the candidate drugs in the prevention Aβ amyloid neuropathology and attenuation of memory deterioration.

Finally, since treatment was delivered in the drinking water, in control studies the inventors rigorously monitored that propranol-HCL, nicardipine-HCL, and losartan did not significantly influence the amount of fluid intake throughout the study period, relative to the control group. In view of the fact that ˜6 month old mice (˜20-23 g in weight) drank ˜4 ml/day of water, the concentration for each drug delivered in the drinking water was calculated such that in this volume, each mouse would receive the final desiderate drug concentration. The evidence that propranol-HCL, nicardipine-HCL, and losartan treatment did not influence water intake is highly relevant and suggests that appropriate doses of drugs were accurately delivered across the entire treatment period.

Feasibility Pharmacokinetic Evidence Suggesting that Certain Antihypertensives can Reach the Brain at nM Concentration and Exert Aβ-Lowering Activity Through Modulation of APP Processing in Tg2576 Mice

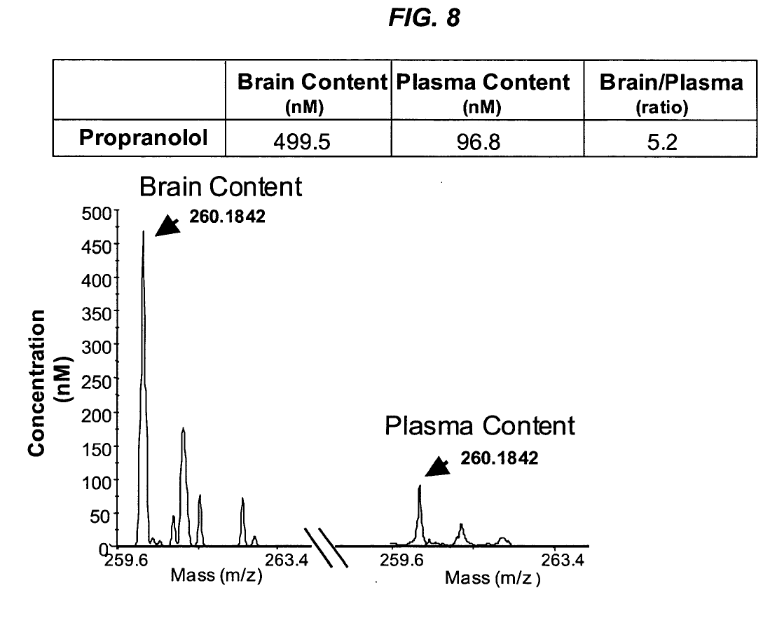

The pharmacokinetic characterization of candidate Aβ-lowering antihypertensives in Tg2576 mice, at doses below those typically prescribed for hypertension, and without hypotensive side effects (see FIG. 7) is of interest. In feasibility studies it was found that short term dosing with propranolol-HCL, resulted in nM levels in the cerebellum of Tg2576 mice, which reached ˜5 fold higher levels than that found in plasma from the same mouse (FIG. 8). This feasibility evidence showing accumulation of propranolol-HCL in the brain strongly supports a “central mechanistic role” for this drug in terms of Aβ-lowering activity. This evidence is very exciting and could result in immediate clinical application for e.g., attenuation of memory deterioration.

In this short-term dosing study, Tg2576 mice were treated with propranolol-HCL for three-weeks at a concentration ˜3 fold lower than used for treatment of hypertension (10 mg/kg/day delivered in the drinking water). At the time of sacrifice, emicerebellum (pool from N=3 per group) was homogenized 0.4 M perchloric acid, centrifuged (3,000×g; 15 min), and the aqueous layer evaporated by heating (Botterblom et al., 1993), while the residual diethyl-ether and acidic aqueous layer was used for analysis. Plasma from propranolol-HCL treated mice (pooled, n=3 per group) was extracted using a modified protocol (Martin et al., 2004) mixing equal volumes of plasma in 0.1% NaOH. Propanolol-HCL was then extracted by ethyl acetate and centrifuged. The acidic aqueous layers were then dried, samples were analyzed by tandem liquid chromatography—mass spectrometry, and concentration was determined against an internal standard, in a range of quantification of n between 1-1,000 ng/ml.

These studies provide proof that propranolol-HCL could potentially alter Aβ1-42 levels in the brain in vivo. Collectively the feasibility studies strongly support the hypothesis that even short exposure of Tg2576 mice to certain antihypertensive drugs, at subclinical doses, should beneficially influence AD type amyloid neuropathology (Aβ peptides), in the absence of detectable side effects.

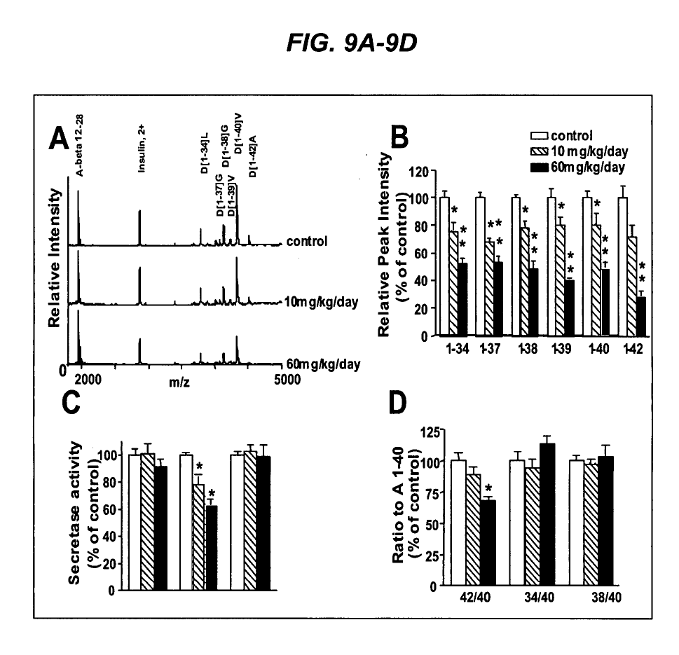

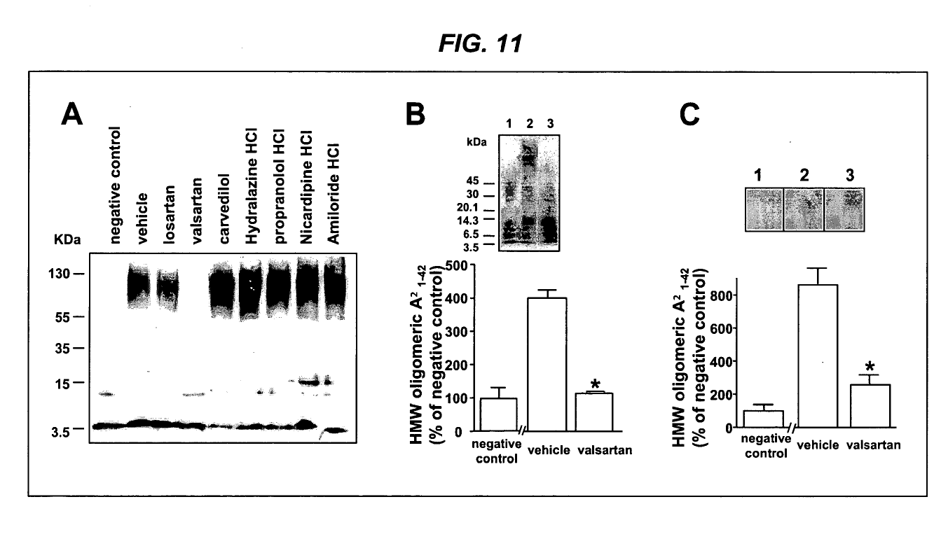

Based on the exciting evidence suggesting that short term dosing with propranolol-HCL, resulted in nM concentration in the brain of Tg2576 mice, in feasibility studies additional studies to explore mechanistically the Aβ lowering activity of propranolol-HCL in the brain were performed. Using immune-precipitation (IP)-mass spectrometry (IP-MS) technique (Wang et al., 2005), it was found that treatment of Tg 2576 mice with propranolol-HCL at 10 or 60 mg/Kg/day for 3 weeks, resulted in dose dependent decrease in Aβ1-42 content in the neocortex as expected (FIG. 9A,B), confirming ELISA assay (see FIG. 11). Most interestingly, it was found that propranolol-HCL treatment also resulted in a dose-dependent reduction of most of the detectable Aβ species in the brain, namely Aβ1-34, Aβ1-37, Aβ1-38, Aβ1-39, Aβ1-40, relative to control untreated Tg2576 mice (FIG. 9B).

In search of potential mechanisms ultimately responsible for the observed overall decline of the detectable Aβ species in the Tg2576 brain, in feasibility studies it was found that treatment with propranolol-HCL coincided with a selective decrease in β-secretase activity assessed by fluorometric based activity assays (R&D Systems; Wang et al, 2005) in the same brain (contralateral neocortex); no detectable change in α- and γ-secretase activity in the brain, relative to untreated Tg2576 mice, was found (FIG. 9C). Most interestingly, it was found that the ratio of Aβ1-42 to Aβ1-40 as % of control was significantly reduced in the propranolol-HCL treated group, while the ratio of Aβ1-34 and Aβ1-38 to Aβ1-40 was unaffected (FIG. 9D). This observation tentatively suggests that in addition of selectively inhibiting APP processing by influencing β-secretase activity (ultimately reducing the generation of multiple Aβ species in the brain), propranolol-HCL may also (directly or indirectly) influence γ-secretase cleavage favoring Aβ1-40 ultimately resulting in relatively lower generation of Aβ1-42 peptides as reflected by a significant decreased Aβ1-42/Aβ1-40 ratio (FIG. 9D). This evidence strongly supports the role of candidate Aβ lowering antihypertensives in short term treatment studies in vitro to be tested in preventive and therapeutic studies.

Chronic Preventive Treatment with Valsartan Attenuates AD-Type Cognitive Deterioration in the Tg2576 AD Mouse Model

In a recent parallel study, the investigation of the potential beneficial role of “chronically” treating with valsartan was performed primarily because of previous evidence that valsartan delivered either at doses below or within those recommended for hypertension in normotensive rodents (Yamamoto et al., 1997) does not influence blood pressure (see below). Based on this consideration, the inventors initiated a “chronic-long-term” preclinical treatment with valsartan in Tg2576 mice at doses ˜2 fold lower or within that prescribed for treatment of hypertension and skipped a short-term dosing study for valsartan as proposed for all the identified antihypertensive-Aβ lowering drugs.

Valsartan has received a great deal of attention, especially in the geriatric population, primarily because of 1) the superior tolerability and safety of ARBs (Unger et al., 1999; Formica et al., 2004), and 2) accumulating evidence that ARBs may protect against end-organ damage such as cardiac hypertrophy and renal disease in hypertensive individuals. Among the 7 antihypertensive drugs that were found herein to reduce Aβ in vitro, valsartan is most commonly prescribed for hypertension. The clinically recommended valsartan dose range for the treatment of hypertension in human is 80-320 mg/day (online Physicians' Desk Reference), which corresponds to ˜20-60 mg/Kg/day in mice, based on calculations using a well-accepted formula for converting drug equivalent dosages across species (Wang et al., 2007). For in vivo studies, we treated Tg2576 mice with 10 or 40 mg/kg/day valsartan, equivalent to, respectively, human doses of 55 and 220 mg/day. These doses correspond, respectively, with ˜2 fold below, or within, the doses prescribed for the treatment of hypertension.

The inventors previously showed in vivo studies in which, young adult Tg2576 mice were chronically treated with valsartan starting at ˜6 months of age, when cognitive deterioration is incipient, despite the fact that AD-type amyloid neuropathology is typically not detectable (Kawarabashi et al, 2001). After ˜5 months of valsartan treatment, mice were assessed for cognitive functions and brain Aβ neuropathology at ˜11 months of age. In control studies, it was found that adult Tg2576 mice were normotensive, compared to age-, gender- and strain-matched wild-type mice (see Example 3 below). Consistent with previous reports that valsartan does not reduce blood pressure in normotensive rats, it was found that chronic (˜5 month) valsartan treatment in normotensive Tg2576 mice did not influence systolic, diastolic, and MAP blood pressure (described in further detail in Example 3).

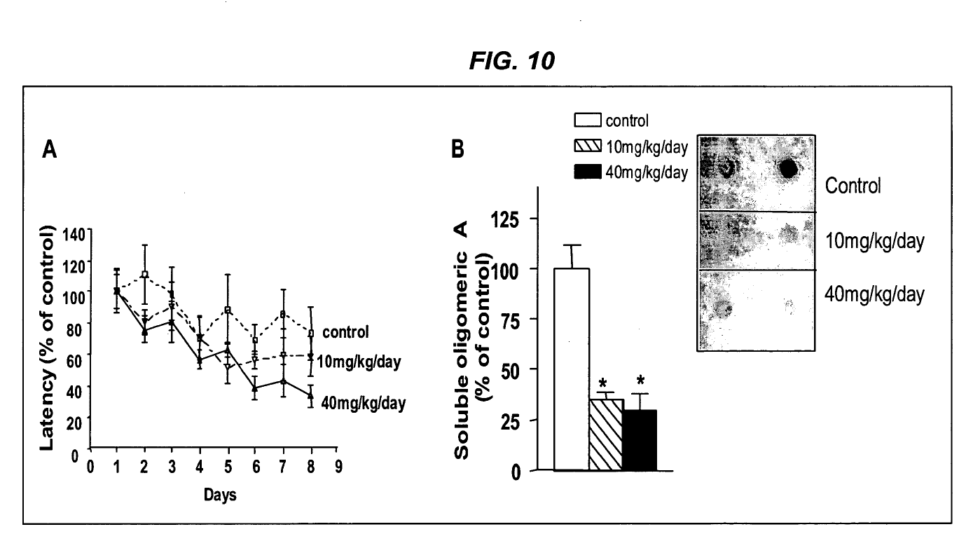

The Tg2576 AD mouse model is well known to develop progressive Aβ-associated cognitive deterioration with increasing age (Hsiao et al., 1996 ). As expected, non-treated control ˜11-month old Tg2576 mice showed impaired acquisition of spatial learning in the Morris water maze cognitive behavioral task. They also failed to learn and use the available visual cues to help localize the submerged escape platform during the learning trials, as evident by the lack of significant improvements in escape latency across consecutive learning trials (FIG. 10A). In contrast, it was found that valsartan-treated Tg2576 mice were able to learn and use the visual cues to help localize the escape platform, as demonstrated by significantly reduced escape latency with progressive learning trials at 10 and 40 mg/kg/day (FIG. 10A).

In this study cognitive behavioral function was assessed using the Morris water maze. Following Morris water maze testing and blood pressure assessment, mice were sacrificed for neuropathological assessment. FIG. 10A) Assessments of spatial memory behavioral performance by Morris water maze paradigm in ˜11-month old control Tg2576 mice (untreated) and in Tg2576 mice which underwent treatment with 10- or 40-mg/kg-day valsartan salt in the drinking water for ˜5 months; n=6-10 per group. Two-way ANOVA; 10 mg/kg/day valsartan treated group vs. non-treated control group, F1,7793=4.913, p<0.00288 for drug treatment, F7,23660=2.131, p<0.0466 for escape latency over time; 40 mg/kg/day valsartan treated group vs. non-treated control group, F1,19840=14.28, p<0.0003 for drug treatment, F7,34510=3.549, p<0.0018 for escape latency over time. FIG. 10B) Assessments of HMW-soluble Aβ peptide contents in the brain using an antibody specific for HMW oligomeric Aβ peptides in a dot blot analysis (B-inset) of representative of HMW-soluble extracellular Aβ contents (McLaurin et al., 2006). Bar graph represents mean±SEM values, n=6-10 mice per group; *P<0.001.

In view of the central role of HMW-soluble Aβ oligomers in AD cognitive dysfunction the inventors continued to explore the potential impact of valsartan treatment on the accumulations of HMW-soluble Aβ peptides in the brain of Tg2576 mice. Using an established dot-blot immunoassay using an antibody that selectively detects HMW oligomeric Aβ species with molecular masses >40 kDa (32), the inventors found that coincidental to cognitive benefit (FIG. 10B), prophylactic valsartan treatments resulted in significant reductions in the contents of HMW-soluble oligomeric Aβ species in the brain of ˜11 months Tg2576 mice (see Example 3 below). Notably, 10 mg/kg/day valsartan treatment equivalent to a human dose ˜2 fold below the recommended therapeutic valsartan dose range for hypertension treatment proved to be efficacious in reducing HMW—soluble oligomeric Aβ accumulation in the brain coincidental with attenuation of cognitive deterioration in the Tg2576 mouse AD model (see Example 3 below).

Thus, the data in this Example support the finding that chronic preventative valsartan treatment attenuates the onset of Aβ related cognitive deterioration in Tg2576 mice, possibly through mechanisms preventing the accumulation of HMW-soluble oligomeric Aβ peptides in the brain, even when delivered at doses lower than that prescribed for the treatment of hypertension. Without being bound to a particular theory or mechanism of action, it is possible that this finding is a result of mechanisms involving 1) reducing Aβ aggregation, and/or 2) by the promotion of Aβ clearance through degradation of Aβ peptides by the membrane-associated insulin degrading enzyme (IDE) as further discussed in Wang et al. (2007). The valsartan mediated potentiation in membrane IDE activity in the brain was rather selective since no detectable changes in APP processing assessed by α- β- or γ-secretase activity fluorometric based activity assays (R&D Systems; Wang et al., 2005) or by detection of carboxy terminal fragments (CTF)-α-β- or γ-were found (Wang et al., 2005), were found. Collectively, this evidence strongly support the studies the use of candidate antihypertensive-Aβ lowering drugs as potential future Aβ lowering drugs as being capable of attenuating cognitive deterioration in preclinical AD and eventually in clinical AD.

Below in Table 6, there are included recommended experimental doses for treatment in Tg2576 mice with carvedilol, amiloride, hydralazine at does ˜3 fold lower (subclinical) or within the range of that used for the treatment of hypertension (clinical dose) in human. It is contemplated that such low doses of these drugs will be useful in attenuating cognitive deterioration in preclinical AD and eventually in clinical AD.

TABLE 6

| | recommended | | | | |
|---|---|---|---|---|---|
| | clinical doses | Tg2576 treatment | | Equivalent human dose | |
| | for hypertension | Clinical | Sub-clinical | Clinical | Sub-clinical |
| | treatment in human | dose | dose | dose | dose |
| | (mg/day) | (mg/kg/day) | (mg/kg/day) | (mg/day) | (mg/day) |
| carvedilol | 120-60 | 17 | 4 | 90 | 20 |
| amiloride | 20-5 | 2.3 | 0.3 | 12 | 1.7 |
| hydralazine | 300-40 | 32 | 2.4 | 170 | 13 |

### Example 3

### Valsartan Lowers Brain β-Amyloid and Improves Spatial Learning in a Mouse Model of Alzheimer's Disease

The above studies show that some antihypertensive medications may reduce the risk for Alzheimer's disease (AD). The inventors screened 55 clinically prescribed antihypertensives for AD-modifying activity using primary cortico-hippocampal neuron cultures generated from the Tg2576 mouse AD model. The agents represented all drug classes used for hypertension pharmacotherapy. 7 antihypertensive agents were identified that significantly reduced AD-type amyloid beta-protein (Aβ) accumulation. Through in vitro studies, it was found that valsartan, one of the seven candidate drugs from the high throughput drug screening, is also capable of attenuating oligomerization of Aβ peptides into high-molecular-weight (HMW)—oligomeric peptides, known to be involved in cognitive deterioration. It was found that preventive treatment of Tg2576 mice with valsartan significantly reduced AD-type neuropathology and the content of soluble HMW extracellular oligomeric Aβ peptides in the brain. Most importantly, valsartan administration also attenuated the development of Aβ-mediated cognitive deterioration, even when delivered at a dose ˜2 fold lower than that used for hypertension treatment in humans. These preclinical studies, for the first time, suggest that certain antihypertensive drugs with AD-modifying activity might protect against progressive Aβ-related memory deficits in AD, or in subjects at high risk of developing AD (e.g., mild cognitive impairment (MCI)).

A high throughput drug screening was performed to test the hypothesis that antihypertensive drugs might influence AD through mechanisms affecting β-amyloid (Aβ) neuropathology, independent of blood pressure-lowering activity. Abnormal accumulations of Aβ peptides in the brain are associated with a cascade of cellular events resulting in cognitive decline (9). Aβ species with different amino and carboxyl termini are generated from the ubiquitously expressed amyloid precursor protein (APP) through sequential proteolysis by β- and γ-secretases. A third proteolytic enzyme, α-secretase, may reduce Aβ generation by cleavage of APP within the Aβ peptide sequence. While aggregation and precipitation of Aβ peptides into extracellular amyloid plaque deposits in the brain are key pathological features of AD, recent studies indicate that accumulations of soluble high molecular-weight (HMW) extracellular oligomeric Aβ species, rather than deposition of amyloid per se, might be specifically related to spatial memory reference deficits.

Methods

Cell Culture and Drug Screening: Embryonic-day (E)16 cortico-hippocampal neuronal cultures were prepared from heterozygous Tg2576 transgenic mice (Tg2576 neurons) (Mirjany, et al., 2002, Journal of Pharmacology and Experimental Therapeutics 301:494-500.) (see below). Embryonic brain tissue was mechanically triturated and centrifuged. Neurons were seeded onto poly-D-lysine-coated 96-well plates at 1.0×105 cells per well and cultured in the serum-free chemically-defined medium Neurobasal, supplemented with 2% B27, 0.5 mM L-glutamine and 1% penicillin-streptomycin (Gibco-BRL). The absence of astrocytes (<2%) was confirmed by the virtual absence of glial fibrillary acidic (GFAP) protein immunostaining.

For primary screening, cultured neurons were treated with 100 μM of drug in duplicates for 16 hours; all drugs were obtained in stock from MicroSource Discovery Systems Inc (Gaylordsville, Conn.). Conditioned medium was collected for Aβ detection using commercially available ELISA kits (BioSource). Drugs that reduced Aβ content by >15% in the primary screening were selected for secondary screening. Primary neurons prepared in 96-well plates were treated with 0.1 μM, 1 μM, 10 μM, 50 μM, and 100 μM of each drug in duplicate for ˜16 hours and conditioned medium was collected for Aβ detection.

Cell viability was assessed using a commercial available LDH assay kit according to the manufacture's instruction (Promega). EC50 values of each drug were calculated by using GraphPad Prism software package (GraphPad Software, Inc., San Diego).

Aβ-peptideoligomerization assay in vitro: Lyophilized Aβ₁₋₄₂ peptide was dissolved in 1,1,1,3,3,3,-hexafluoro-2-propanol (HFIP, from Sigma), incubated at room temperature for 60 min, aliquoted, vacuum dried and stored at −80° C. Aβ peptide was dissolved in DMSO and diluted into ddH2O to a final concentration of 100 μg/ml. The peptide was then mixed with equal volume of drugs and incubated at 37° C. for 1 day (Klein, W. L. 2002. Neurochemistry International 41:345-352).

Following incubation, samples were centrifuged at 14,000×g for 10 min at 4° C. Supernatants were mixed with 2× SDS sample buffer and separated on a 10-20% Tris-Tricine gradient SDS gel (Invitrogen). The separated peptides were subjected to Western blot using 6E10 antibody (1:1000, Signet, St. Louis). Immunoreactive signals were visualized by using enhanced chemiluminescence detection (Amersham), and quantified densitometrically (Quantity One, Bio-Rad).

For dot blot analysis, samples used for the Western blot analysis (100 ng peptide) were directly applied to the nitrocellulose membrane, air dried and blocked with 5% non-fat milk followed by incubation with antibody A11 (Biosource, Camarillo, Calif.), an antibody that specifically recognizes the oligomeric form of Aβ. Immunoreactive signals were detected and quantified as described above.

Tg2576 mice and valsartan treatment: This study used Tg2576 AD transgenic mice that express the human 695-amino acid isoform of APP, containing the Swedish double mutation (APP_{swe}) [(APP695) Lys670→Asn, Met671→Leu] driven by a hamster prior promoter. Female Tg2576 mice and age, gender, and strain-matched WT mice (Taconic, Inc) were randomly assigned to the following valsartan treatment groups: 10 mg/kg/day, 40 mg/kg/day, and the control water treatment group. Animals were treated at 7 months of age.

Valsartan mono-sodium salt was obtained from MicroSource Discovery Systems Inc (Gaylordsville, Conn.). For the preparation of valsartan drinking solutions, the inventors dissolved valsartan (stored in dry environment) in sterile water by adding valsartan salt to water at 30-40° C. and stirred vigorously until completely dissolved. The solution was then cooled to room temperature slowly without external cooling to discourage precipitation of the drug. Valsartan salt has a solubility of ˜5 g/L at room temperature and the aqueous valsartan salt solution is slightly acidic, with a pH of 5.5. Aqueous valsartan solution was neutralized with sodium bicarbonate without detectable reduction of valsartan solubility. For our preclinical in vivo studies, we prepared neutralized valsartan aqueous solutions at concentrations (50-200 mg/L) well below the maximal solubility of sodium valsartan in water. Valsartan solutions in the drinking water were wrapped in aluminum to avoid potential photochemical changes and always maintained at room temperature to avoid potential precipitation from solution. Valsartan salt does not contain labile groups, and routine quality control checking found no change in the recovered compound on TLC analysis at both 50 and 200 mg/L valsartan solutions. Quality was assessed in 3-10 week old solutions stored at room temperature in dark compartments. Drinking solutions for the in vivo treatments were freshly prepared twice a week. Liquid consumption and animal body weight were monitored weekly throughout the study.

At ˜11 months of age, following assessment of spatial memory functions by the Morris water maze test, mice were anesthetized with the general inhalation anesthetic 1-chloro-2,2,2-trifluoroethyl difluoromethyl ether (Baxter Healthcare, Deerfield, Ill.) and sacrificed by decapitation. Brains were harvested and hemi-dissected. One hemisphere was fixed in 4% paraformaldehyde for 24 hours for histological studies. Hippocampus and cortex were dissected from the opposite hemisphere, rapidly frozen, pulverized in liquid nitrogen, and stored at −80° C. for biochemical studies.

Assessment of blood pressure and glucose utilization. Mouse blood pressure was routinely recorded using a commercial blood pressure analysis system designed specifically for small rodents (Hatteras Instruments, NC). To assess potential alteration in glucose utilization in response to chronic treatment with valsartan, an insulin glucose tolerance test (IGTT), was used as previously described. Briefly, mice were given a single dose of glucose post-prandially (i.p. 2 g/kg body weight). Blood was collected from the tail-vein periodically over a 2-hour period. Blood glycemic content was assessed using the OneTouch LifeScan System, (LifeScan, Milpitas, Calif.) following the manufacturer's instruction.

Behavioral assessment of cognitive function by the Morris water maze test. The Morris water maze test was used to evaluate working and reference memory function in response to treatment with valsartan in Tg2576 mice, as previously described (Morris, R. 1984. Journal of Neuroscience Methods 11:47-60.). At ˜11 months of age, mice were put into the water maze from 4 different quadrants; spatial memory was assessed by recording the average latency time for the animal to escape to the hidden platform. The behavior analysis was consistently conducted during the last 4 hours of the day portion of the light cycle in an environment with minimal stimuli (e.g., noise, movement, or changes in light or temperature).

Assessment of HMW-soluble oligomeric Aβ-oligomerization in the brain by dot-blot assay. In this study, soluble proteins were extracted from the brain samples (cortex) with PBS in the presence of protease inhibitors and centrifuged at 78,500×g for 1 hour at 4° C. (32). 4 μg of extracellular soluble protein isolated from the cerebral cortex of valsartan- or vehicle-treated Tg2576 mice was directly applied to a nitrocellulose membrane, air dried, and blocked with 5% non-fat milk, as previously described (McLaurin et al., 2006. Cyclohexanehexol inhibitors of A[beta] aggregation prevent and reverse Alzheimer phenotype in a mouse model. Nat Med advanced online publication.). The membrane was probed with either anti-oligomer A11 antibody (1:1000, Biosource) or 6E10 antibody (1:1000, Signet, St. Louis) (McLaurin et al., 2006. Cyclohexanehexol inhibitors of A[beta] aggregation prevent and reverse Alzheimer phenotype in a mouse model. Nat Med advanced online publication). Dot blot immunoreactivities were quantified densitometrically. For total soluble Aβ assessment, the extracellular soluble protein used for dot blot was subject to ELISA analysis (BioSource, Camarillo, Calif.) (Wang, et al., 2005, FASEB J. 04-3182fje).

Assessment of AD-type amyloid neuropathology in Tg2576 mice. For quantitative assessment of Aβ peptide in the brain, frozen pulverized tissue was homogenized in 5.0 M guanidine buffer, diluted (1:10) in phosphate-buffered saline containing 0.05% (v/v) Tween-20 and 1 mM Pefabloc protease inhibitors (Roche Biochemicals, Indianapolis, Ind.), and centrifuged for 20 min at 4° C. Total Aβ1-40 or Aβ1-42 was quantified by sandwich ELISA (BioSource, Camarillo, Calif.), as previously reported (Wang, et al., 2005, FASEB J. 04-3182fje). Serum Aβ content was analyzed using the same ELISA Kit, following manufacturer instructions.

For stereological assessment of AD-type amyloid burden, freshly harvested mouse brain hemispheres were immersion fixed overnight in 4% paraformaldehyde. They were then sectioned in the coronal plane on a Vibratome at a nominal thickness of 50 μm. Every 12th section was selected from a random start position and processed for thioflavin-S staining, as previously described (Wang, et al., 2005, FASEB J. 04-3182fje; Vallet, et al., 1992. A Acta Neuropathologica 83:170-178). All stereologic analysis was performed using a Zeiss Axiophoto photomicroscope equipped with a Zeiss motorized stage and MSP65 stage controller, a high-resolution—MicroFire digital camera, and a Dell computer running the custom designed software Stereo Investigate. The amyloid burden was estimated using the Cavalieri principle with a small size grid (25×25 μm) for point counting, as previously described (Wang, et al., 2005, FASEB J. 04-3182fje).

APP processing and α, β, γ-secretase activity: Expression of holo-APP was examined by Western blot analysis with the C8 antibody (raised against AA 676-695 of human APP cytoplasmic domain; gift of Dr. Dennis Selkoe, Brigham and Women's Hospital). α- β- and γ-secretase activities were assessed using commercially available kits (R & D Systems, Minneapolis, Minn.) (Wang, et al., 2005, FASEB J. 04-3182fje; Ho et al. 2004. FASEB J. 03-0978fje.). Brain samples were homogenized in supplied buffers. Homogenate was then added to secretase-specific APP peptide conjugated to the reporter molecules EDANS and DABCYL. In the uncleaved form, fluorescent emissions from EDANS were quenched by the physical proximity of the DABCYL moiety, which exhibit maximal absorption at the same wavelength (495 nm). Cleavage of APP peptide by secretase physically separates the EDANS and DABCYL reporter molecules, allowing for the release of a fluorescent signal. The level of secretase enzymatic activity is proportional to the fluorometric reaction in the homogenate (R & D Systems).

Insulin degrading enzyme (IDE) protein content and enzymatic activity assay: Frozen brain samples were pulverized in dry ice and homogenized in homogenization buffer (50 mM HEPES pH 7.4, 100 mM NaCl, sigma protease inhibitor 20 μl/g tissue) by passing them through a 26-gauge needle ˜15 times. Lysates were first centrifuged at 2,500×g, 15 min at 4° C. to remove nuclei and cell debris and subsequently at 100,000×g at 4° C. for 60 min, to separate the post-nuclear membrane fraction (pellet) and cytosolic fraction (supernatant) (Qiu, et al., 1998. J. Biol. Chem. 273:32730-32738.). Fractions were then separated (25-30 μg proteins) on 10% SDS-PAGE and subjected to western blot analysis using rabbit anti-mouse IDE antibody (Abcam Inc, Cambridge, Mass.). IDE immunoreactivity was visualized with ECL (Amersham) and quantified autoradiographically. Actin immunoreactivities (rabbit anti-actin, 1:5000, Sigma, St. Louis, Mo.) were used to control sample loading and normalization of IDE immunoreactive signal.

IDE activity was measured by degradation of 125I-insulin, as previously described, with modifications (Qiu, et al., 1998. J. Biol. Chem. 273:32730-32738; Zhao et al. 2005. FASEB J. 05-4359fje.). Briefly, the same protein fractions (50 μg) used for assessment of IDE protein expression were incubated in the presence of 125I-insulin in reaction buffer (50 mM Hepes pH 7.4, 100 mM NaCl, sigma protease inhibitor 20 μl/g tissue and 1% BSA) at 37° C.; the reaction was stopped by adding 9 volumes of 5% TCA. The assessment of 125I-insulin released into the TCA-soluble, degraded insulin or TCA-insoluble, un-degraded insulin were used as IDE activity indexes.

Endothelin-Converting Enzyme Activity and Neprilysin Protein Content.

Frozen pulverized brain samples were homogenized in homogenization buffer (50 mM Tris/pH6.8, 0.1 mM PMSF); nuclei and cell debris were removed by centrifugation at 2,500×g for 15 minutes. The membrane pellet was obtained by centrifugation at 100,000×g for 45 minutes. The obtained membranes were washed once and dissolved in the homogenization buffer supplied with 1% N-octyl-glucoside (Sigma) for 1 hour at 4° C. The non-soluble part was removed by centrifugation at 20,000×g for 60 minutes. Protein content in the supernate was measured using a Bio-Rad Protein Assay. 50 μg of the membrane protein was incubated with 100 ng rat big ET-1 (America peptide Co.) at 37° C. for 4 hours in 250 μl of the reaction mixture (50 mM Tris/pH7). The reaction was stopped by adding 600 μl of cold ethanol (−20° C.). After centrifugation at 10,000×g for 10 minutes, the resulting supernate was lyophilized and the dry pellet was reconstituted with 250 μl of the assay buffer and subjected to the ET-1 measurement by an Endothelin-1 Biotrak ELISA System (Amersham Biosciences, UK). A cubic-spline curve was fitted to the standards and the unknown values were interpolated from the standard curve (Lopez-Ongil S et al. 2005. Cell Physiol Biochem 15:135-44.; Takahashi et al., 1995. Biochem J 311:657-65.).

Zinc-dependent metalloprotease neprilysin (NEP) content in the mouse brain was measured by western analysis using rabbit anti-mouse NEP antibody (Alpha Diagnostic International, Texas).

Results and Discussion

The present Example shows that certain antihypertensive drugs are able to lower Aβ in vitro. It was also found that the angiotensin-II type-1 receptor blocker (ARB) valsartan is able to lower Aβ and inhibit Aβ oligomerization into soluble HMW extracellular species in vivo. These effects were seen even at a dose equivalent to ˜2 fold lower than that commonly prescribed for the treatment of hypertension in humans. The functional relevance of this finding was confirmed by evidence that valsartan's Aβ-lowering activity in the brain coincided with attenuation of spatial memory reference deficits in Tg2576 mice, in the absence of detectable blood pressure-lowering activity.

The high throughput screening study assessed 55 antihypertensive drugs representing all pharmacological classes of currently available antihypertensives (Table III above in Example 2). It was found that 7 of the 55 agents significantly reduced the accumulation of Aβ1-40 and Aβ1-42 in primary embryonic cortico-hippocampal neuron cultures derived from the Tg2576 mouse AD model. Aβ reductions were observed in a dose-dependent fashion (Table IV above in Example 2). The predicted drug concentrations resulting in a 50% inhibition of steady-state Aβ peptide levels (EC50) in the conditioned medium were calculated at μM range (Table IV above in Example 2). No neurotoxicity was detected with any of the 7 agents, as assessed by lactate dehydrogenase (LDH) activity at drug concentrations up to 10 μM (Table IV above in Example 2).

The 7 Aβ-lowering antihypertensive agents found to be effective in attenuating the accumulation of Aβ1-40 and Aβ1-42 are not specific to any single pharmacological class or clinical indication (Table III above in Example 2). The drugs belong to 6 different pharmacological subclasses, all of which are prescribed for the treatment of hypertension: 1) propranolol-HCL, β-adrenergic blocker, 2) carvedilol, β/α-adrenergic blocker, 3) valsartan and losartan, angiotensin-II type-1 receptor blockers (ARBs), 4) nicardipine-HCL, Ca++-channel blocker, 5) amiloride, K+-sparing diuretic, and 6) hydralazine-HCL, vasodilator (Table 1III above in Example 2).

Recent evidence indicates that spatial memory reference deficits in Tg2576 mice are primarily influenced by the accumulation of soluble, extracellular HMW-Aβ species, rather than the deposition of total guanidine-extractable Aβ peptides in the AD-type amyloid plaques in the brain (Lesne et al., 2006, Nature 440:352-7). Based on this evidence, the inventors investigated the role of the 7 identified anti-hypertensive drugs in preventing Aβ oligomerization into soluble HMW species in vitro.

Using an established in vitro oligomerization assay (McLaurinet al. 2006. Cyclohexanehexol inhibitors of A[beta] aggregation prevent and reverse Alzheimer phenotype in a mouse model. Nat Med advanced online publication.), it was found that valsartan (10 μM) significantly prevented, by >5 fold, the oligomerization of Aβ1-42 (and Aβ1-40) into >40 kDa HMW Aβ-peptide species relative to vehicle-treated control peptides, as assessed by Western Blot assay in vitro, (FIG. 11A). Consistent with this evidence, using a dot-blot immunoassay with an antibody (A11) that selectively detects HMW Aβ species with molecular masses >40 kDa (McLaurin et al., 2002 Nat Med 8:1263-1269.), it was found that valsartan prevented Aβ1-42 oligomerization in vitro, relative to vehicle-treated peptides (FIG. 11B), 24 hours after incubation.

Compared with other antihypertensive compounds that were found to lower Aβ, valsartan had a qualitatively stronger in vitro anti-Aβ oligomerization activity. Because of this consideration and the good tolerability and safety record of valsartan in the treatment of hypertension, the inventors proceeded with a series of in vivo studies to assess any functional beneficial role of the agent in preventing AD-type spatial memory reference deficits and Aβ-neuropathology in adult Tg2576 mice.

Chronic Valsartan Treatment is Well Tolerated in Tg 2576 Mice

The recommended dose of valsartan for the treatment of hypertension in humans is 80-320 mg/day (online Physicians' Desk Reference). This range corresponds to ˜20-60 mg/kg/day in mouse, as derived using FDA criteria for converting drug equivalent dosages across species, based on body surface area ([human equivalent dose in mg/kg=animal dose in mg/kg×(animal weight in kg/human weight in kg)0.33]). Because the overall goal of the study was to test the hypothesis that certain antihypertensive drugs may influence AD-type amyloid pathogenesis independent of blood pressure-lowering activity, the inventors treated Tg2576 mice with either 10 or 40 mg/kg/day of valsartan, doses either ˜2 fold below, or within the recommended human-equivalent dosage range (55 and 220 mg/day, respectively).

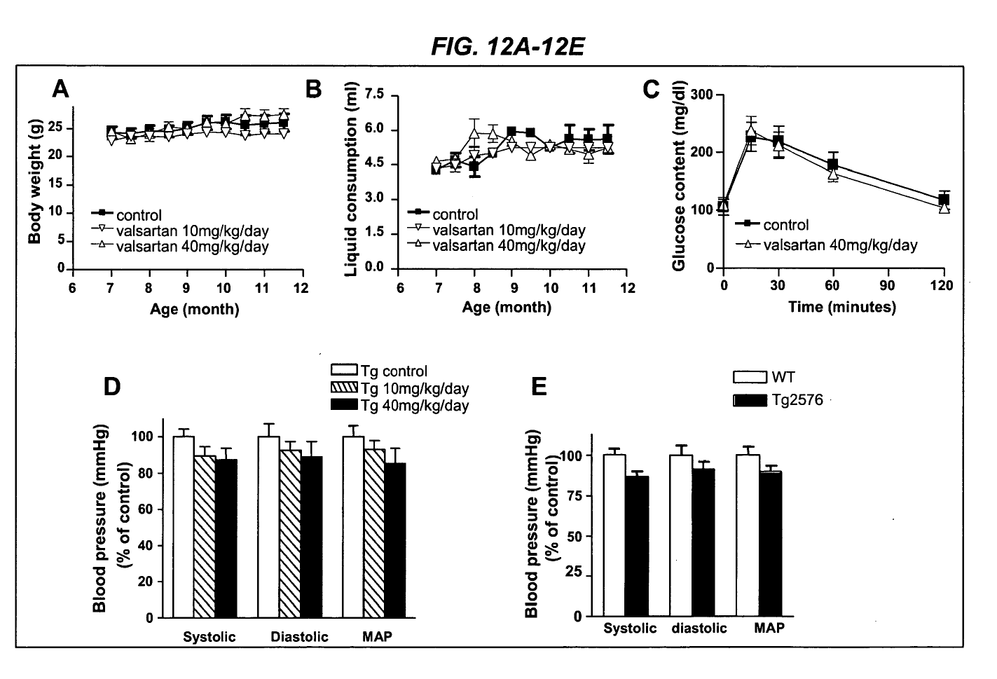

Chronic valsartan treatment, e.g., for ˜5 months in Tg2576 mice, delivered in the drinking water at either 10 or 40 mg/kg/day, did not significantly influence animal body weight (FIG. 12A), daily fluid consumption (FIG. 12B), or general metabolic status, as reflected by glucose-tolerance responses (FIG. 12C), assessed at ˜11 months of age.

When the potential influence of valsartan on modifications in blood pressure in Tg2576 mice was tested, it was found that valsartan delivered at either 10 or 40 mg/kg/day failed to produce a statistically significant change of either systolic, diastolic, or mean arterial (MAP) blood pressure in “normotensive” Tg2576 mice (see below), relative to untreated age- and gender-matched Tg2576 control mice, after ˜5 months of chronic treatment (FIG. 12D). These data are consistent with a previous report that valsartan has no effect on normotensive rats.

In further control studies, it was confirmed that adult (6-7 months old) Tg2576 mice are normotensive compared to strain-, age-, and gender-matched wild-type mice (FIG. 12E).

Valsartan Treatment Attenuates AD-Type Cognitive Deterioration Coincidental with the Prevention of Aβ Oligomerization into Soluble HMW Extracellular Species

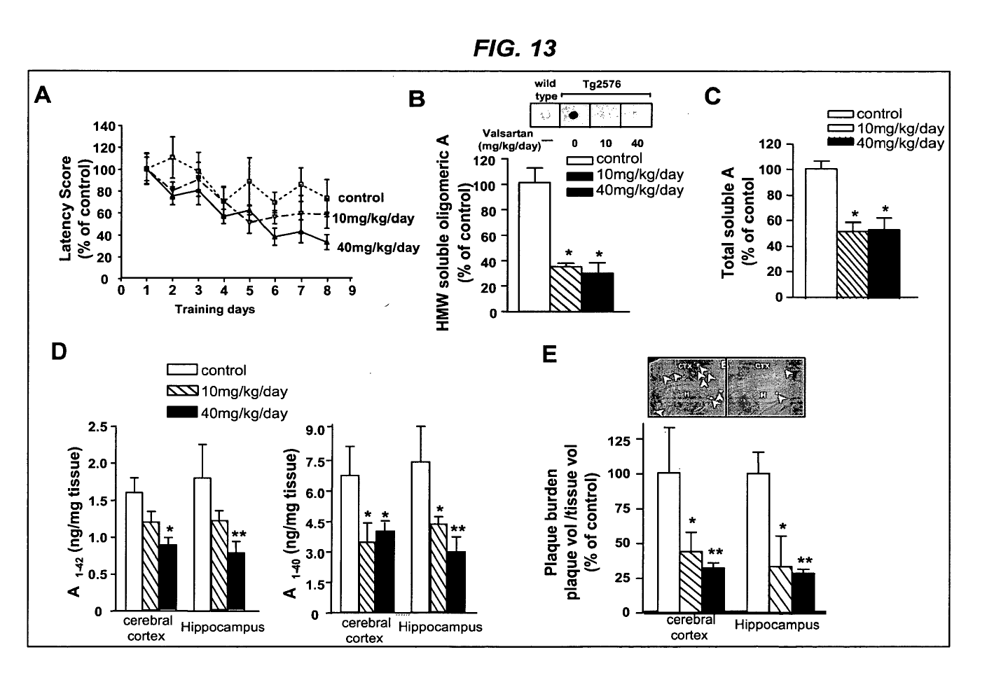

The Tg2576 AD mouse model is well known to develop progressive Aβ-associated cognitive deterioration with increasing age. The results in the present Example demonstrated that untreated control, 11-month old Tg2576 mice showed impaired acquisition of spatial learning, as assessed by the Morris water maze (MWM) test. The mice failed to learn to use the available visual cues to help locate a submerged escape platform, as indicated by the lack of significant improvements in the escape latency across consecutive learning trials (FIG. 13A).

After treatment with valsartan, Tg2576 mice were able to locate the escape platform, as demonstrated by significantly reduced escape latency with progressive learning trials (FIG. 13A), even when delivered at dose equivalents ˜2 fold lower that those commonly prescribed for the treatment of hypertension in humans (10 mg/kg/day) (F1,7793=4.913, P=0.0288 for drug treatment, F7,23660=2.131, P=0.0466 for escape latency).

Tg2576 mice treated with 40 mg/kg/day of valsartan, a dose equivalent to that commonly used for the treatment of hypertension in humans, also performed significantly better than untreated mice (F1,19840=14.28, P=0.0003 for drug treatment, F7,34510 =3.549, P=0.0018 for escape latency) (FIG. 13A). The two valsartan treated groups (10 mg/kg/day vs. 40 mg/kg/day) did not show significant differences in their watermaze behavior test performance (P=0.08).

Treatment of strain-, age-, and gender-matched wild-type (WT) mice with 10 or 40 mg/kg/day valsartan for ˜5 months failed to influence spatial reference memory performance on the MWM test, compared to untreated control WT mice. This finding shows that valsartan may benefit spatial memory reference deficits in Tg2576 mice selectively, through the attenuation of AD-type Aβ-mediated response in the brain.

Given the central role of soluble HMW extracellular Aβ oligomers in AD-type cognitive deterioration, the inventors examined the accumulation of HMW-Aβ peptides in the brain. It was found that treatment of Tg 2576 mice with valsartan, either at 10 or at 40 mg/kg/day, resulted in a ˜2-3 fold reduction in HMW Aβ oligomers in the cerebral cortex (FIG. 13B), ˜5 months after treatment. It was also found that there was a significant reduction of total soluble Aβ peptide in the valsartan treated mouse brains (FIG. 13C).

While it is possible that the observed reduction in soluble, extracellular, HMW-Aβ oligomeric peptide content might be a reflection of an overall reduction in total Aβ peptide (see below, FIG. 13D), it is note that the ratio of soluble HMW-Aβ to total soluble Aβ content in the brain of valsartan-treated Tg2576 is ˜2 fold lower than the untreated Tg2576 animals, suggesting that a significant proportion of the total soluble Aβ peptides in the brain of the valsartan treated groups is not in the neurotoxic soluble, extracellular HMW form. This evidence supports the potential anti-Aβ-oligomeric role of valsartan.

Valsartan Prevents AD-Type Amyloid Neuropathology

Treatment of Tg2576 mice with 40 mg/kg/day valsartan resulted in a 1-2 fold reduction in total guanidine-extractable Aβ1-42 peptide (p<0.05) and Aβ1-40 peptide (p<0.05) in the cerebral cortex and hippocampal formation (p<0.05 and p<0.01 for Aβ1-42 and Aβ1-40 respectively) (FIG. 13D). Treatment with 10 mg/kg/day valsartan also reduced total Aβ peptides accumulation in the brain compared to the untreated control animals (P<0.05 for both Aβ1-42 and Aβ1-40) in the hippocampal formation (FIG. 13D).

Finally, consistent with this evidence that valsartan treatment prevents the accumulation of total Aβ peptides in the brain, a significant reduction in AD-type amyloid plaque burden was seen in the contralateral hemisphere of the same Tg2576 mice treated with valsartan at 10 mg/kg/day (P<0.01 for cortex, p<0.05 for hippocampus) or 40 mg/kg/day (p<0.01 for cortex; p<0.05 for hippocampus), relative to age- and gender-matched untreated control Tg2576 mice, as assessed stereologically (FIG. 13E).

Valsartan may Beneficially Influence AD-Pathogenesis Through Degradation and Clearance of Aβ from the Brain

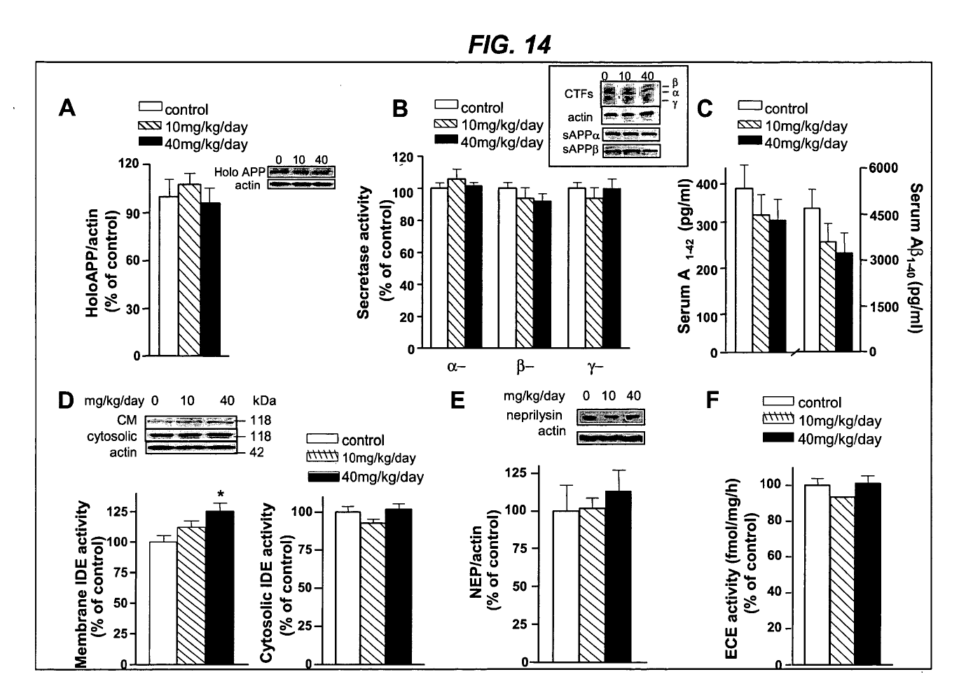

To understand the mechanisms underlying the benefits of valsartan on AD-type cognitive function and Aβ neuropathology, the inventors first examined whether valsartan can influence the processing of the amyloid precursor protein (APP). They found that valsartan treatment has no effect on APP holoprotein levels (C8 immunoreactive) in brain homogenates (cerebral cortex) (FIG. 14A). Also, α-, β-, and γ-secretase activities in the cerebral cortex of valsartan treated Tg2576 mice did not differ from those of age- and gender-matched untreated control Tg2576 mice (FIG. 14B). Consistent with the evidence that valsartan has no effect on the secretase activities, there were no significant changes in the amount of α-, β- or γ-CTFs in the brain of the valsartan treated animals compare to the control animals (FIG. 14. inset), nor were there any significant alterations in the level of sAPPα or sAPPβ. This evidence precludes the possibility that decreased APP processing, and eventually reduced Aβ generation in the brain, could be a mechanism through which valsartan prevents AD-type cognitive deterioration and AD-type neuropathology.

Based on the valsartan-induced decrease in total Aβ1-40 or Aβ1-42 content in the brain, independent of mechanisms involving APP processing, the inventors considered the possibility that valsartan treatment could lead to reduced levels of Aβ peptides in the serum, thereby creating a “sink effect” that promotes efflux of Aβ from the brain into the circulation. There was a 20-25% dose-dependent reduction of Aβ1-42 and Aβ1-40 in the serum of valsartan-treated Tg2576 mice, relative to untreated controls (FIG. 14C), but these changes did not reach statistical significance. It is still possible that valsartan treatment reduces the accumulation of Aβ peptides, including soluble extracellular HMW Aβ oligomers in the brain, in part by promoting peripheral Aβ clearance.

While valsartan showed no influence on secretase activity or APP processing, we did find that valsartan treatment at 40 mg/kg/day lead to a significant elevation in the activity of cell membrane (CM)-bound insulin degrading enzyme (IDE), in the cerebral cortex (P=0.021 1) (FIG. 14D) accompanied by elevation of IDE protein content (FIG. 14D inset), relative to age- and gender-matched untreated control Tg2576 mice. This elevation was highly selective, as there were no detectable changes in intracellular soluble IDE (FIG. 14D), neprilysin (FIG. 14E) or endothelin-converting enzyme (ECE)-1 (FIG. 14F) in the brains of valsartan treated Tg2576 mice, relative to age- and gender-matched Tg2576 untreated control mice. Based on recent evidence showing that AD dementia is associated with reduced membrane IDE activity and content, but not soluble IDE, the observation presented herein suggest that valsartan treatment reduces total Aβ content, including HMW-soluble Aβ in the brain, in part, by facilitating membrane-associated IDE-mediated proteolytic cleavage of Aβ peptides.

This Example was designed primarily in response to a series of epidemiological and clinical studies reporting mixed results on the association of the use of antihypertensive drugs and AD incidence. To clarify whether any of the currently available antihypertensive medications could provide beneficial AD-modifying activity, we surveyed 55 antihypertensive drugs for their potential beneficial role in AD-type amyloid neuropathology. Seven of these were identified to significantly reduce Aβ protein accumulation in vitro, and one, valsartan, was also capable of attenuating oligomerization of Aβ peptides into soluble HMW oligomeric Aβ species in vitro.

The present example shows that valsartan treatments prevent Aβ-related spatial memory reference deficits and AD-type neuropathology in vivo at doses equivalent to or lower than the recommended doses for humans. Valsartan prevents Aβ oligomerization into extracellular soluble HMW species in the brains of Tg2576, even at a dose lower than the clinically recommended dose for hypertensive treatment (10 mg/kg/day). This could be one of the mechanisms through which valsartan prevents Aβ-related spatial memory reference deficits. This scenario is consistent with the recent study showing that intracellular, soluble HMW oligomeric Aβ peptides purified from the brain of middle aged, impaired Tg2576 mice could disrupt memory functions, even episodically, when administered to normal rats.

While valsartan had no effect on APP processing by α-, β-, or γ-secretases, it promoted CM associated IDE-activity in the cerebral cortex. This increase in CM-IDE activity was highly selective, as there were no detectable changes in other proteases involved in the clearance of Aβ (e.g. neprilysin and ECE). Our observation suggests that valsartan treatment might reduce total Aβ including HMW-soluble Aβ in the brain by facilitating cell membrane-associated IDE mediated proteolytic cleavage of Aβ peptides.

Valsartan beneficially prevented Aβ-related spatial memory reference deficit in Tg2576 mice at a dose less than the equivalent recommended clinical dose for hypertensive treatment. These data provide evidence for clinical trials for the use of valsartan in the treatment vulnerable human subjects, such as MCI patients for treating or alleviating the incidence or progression of cognitive impairment in such patients.

All of the compositions and/or methods disclosed and claimed herein can be made and executed without undue experimentation in light of the present disclosure. While the compositions and methods of this invention have been described in terms of specific embodiments, it will be apparent to those of skill in the art that variations of the compositions and/or methods and in the steps or in the sequence of steps of the method described herein can be made without departing from the concept, spirit and scope of the invention. More specifically, it will be apparent that certain agents which are both chemically and physiologically related may be substituted for the agents described herein while the same or similar results are achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference. At certain points throughout the specification, references are referred to using an number in square brackets. Those numbers correspond to the following list of references, each of which is incorporated herein by reference:

Buee L, H of P R, Bouras C, Delacourte A, Perl D P, Morrison J H, Fillit H M. Pathological alterations of the cerebral microvasculature in Alzheimer's disease and related dementing disorders. Acta Neuropathol 87:469-480 (1994)

Burrell L M, Johnston C I. Angiotensin II receptor antagonists. Potential in elderly patients with cardiovascular disease. Drugs Aging 10:421-34 (1997)

Calabuig C, Anton-Fos G M, Galvez J, Garcia-Domenech R. New hypoglycaemic agents selected by molecular topology. Int J Pharm 278:111-118 (2004)

Candido R, Jandeleit-Dahm K A, Cao Z, Nesteroff S P, Burns W C, Twigg S M, Dilley R J, Cooper M E, Allen T J. Prevention of accelerated atherosclerosis by angiotensin-converting enzyme inhibition in diabetic apolipoprotein E-deficient mice. Circulation 106:246-53 (2002)

Chalmers J, MacMahon S. Perindopril protection against recurrent stroke study (progress): interpretation and implementation. J Hypertens Suppl. 21:S9-14 (2003)

Chiolero A, Burnier M. Pharmacology of valsartan, an angiotensin II receptor antagonist. Expert Opin Investig Drugs 7:1915-25 (1998)

Christie R, Yamada M, Moskowitz M, Hyman B. Structural and functional disruption of vascular smooth muscle cells in a transgenic mouse model of amyloid angiopathy. Am J Pathol 158:1065-71 (2001)

Conte V, Uryu K, Fujimoto S, Yao Y, Rokach J, Longhi L, Trojanowski J Q, Lee V M, McIntosh T K, Pratico D. Vitamion E reduces amyloidosis and improves cognitive function in Tg2576 mice following repetitive cincussive brain injury. J Neurochem. 90:758-64 (2004)

Cummings, J. L. Alzheimer's disease. N Engl J Med 351:56-67 (2004)

de Julian-Ortiz J V, de Gregorio Alapont C, Rios-Santamarina I, Garcia-Domenech R, Galvez J. Prediction of properties of chiral compounds by molecular topology. J Mol Graph Model 16:14-18 (1998)

de Julian-Ortiz J V, Galvez J, Munoz-Collado C, Garcia-Domenech R, Gimeno-Cardona C. Virtual combinatorial syntheses and computational screening of new potential anti-herpes compounds. J Med Chem 42:3308-3314 (1999)

Den Heijer T, Skoog I, Oudkerk M, de Leeuw F E, de Groot J C, Hofman A, Breteler M. Association between blood pressure levels over time and brain atrophy in the elderly. Neurobiol Aging 307-313 (2003)

Duan H, Weame S L, Morrison J H, Hof P R. Quantitative analysis of the dendritic morphology of corticocortical projection neurons in the macaque monkey association cortex. Neuroscience 114:349-59 (2002)

Duan H, Wearne S L, Rocher A B, Macedo A, Morrison J H, Hof P R. Age-related dendritic and spine changes in corticocortically projecting neurons in macaque monkeys. Cereb Cortex 13:950-61 (2003)

Duart M J, Garcia-Domenech R, Anton-Fos G M, Galvez J. Optimization of a mathematical topological pattern for the prediction of antihystaminic activity. J Comput Aided Mol Des. 15:561-72 (2001)

Duff K. Eckman C, Zehr C, Yu X, Prada C M, Perez-Tur J, Hutton M, Buee L, Harigaya Y, Yager D, Morgan D, Gordon M N, Holcomb L, Refolo L, Zenk B, Hardy J, Youkin S. Increased amyloid-β42(43) in brains of mice expressing mutant presenilin 1. Nature 383:710-713 (1996)

Emery V O, Gillie E X, Smith J A. Interface between vascular dementia and Alzheimer's syndrome. Nosologic redefinition. Ann NY Acad Sci 903:229-238 (2000)

Eriksen J L, Sagi S A, Smith T E, Weggen S, Das P, McLendon D C, Ozols V V, Jessing K W, Zavitz K H, Koo E H, Golde T E. NSAIDs and enantiomers of flurbiprofen target gamma-secretase and lower Aβ 42 in vivo. J. Clin. Invest. 112:440-449 (2003)

Fogari R, Mugellini A, Zoppi A, Marasi G, Pasotti C, Poletti L, Rinaldi A, Preti P. Effects of valsartan compared with enalapril on blood pressure and cognitive function in elderly patients with essential hypertension. Eur J Clin Pharmacol 59:863-868 (2004)

Forette F, Seux M L, Staessen J A, Thijs L, Babarskiene M R, Babeanu S, Bossini A, Fagard R, Gil-Extremera B, Laks T, Kobalava Z, Sarti C, Tuomilehto J, Vanhanen H, Webster J, Yodfat Y, Birkenhager W H. The prevention of dementia with antihypertensive treatment. New evidence from the Systolic Hypertension in Europe (Syst-Eur) study. Arch Intern Med 162:2046-2052 (2002)

Forette F, Seux M L, Staessen J A, Thijs L, Birkenhager W H, Babarskiene M R, Babeanu S, Bossini A, Gil-Extremera B, Girerd X, Laks T, Lilov E, Moisseyev V, Toumilehto J, Vanhanen H, Webster J, Yodfat Y, Fagard R. Prevention of dementia in randomized double-blind placebo-controlled Systolic Hypertension in Europe (Syst-Eur) trial. Lancet 352:1347-1351 (1998)

Frishman W H. Are antihypertensive agents protective against dementia? A review of clinical and preclinical data. Heart Dis. 4:380-6 (2002)

Galvez J, de Julian-Ortiz J V, Garcia-Domenech R. General topological patterns of known drugs. J Mol Graph Model 20:84-94 (2001)

Galvez J, Garcia R, Salabert M T, Soler R. Charge indexes. New topological descriptor. J Chem Inf Comput Sci 34:520-525 (1994)

Galvez J, Garcia-Domenech R, de Gregorio Alapont C, de Julian-Ortiz J V, Popa L. Pharmacological distribution diagrams: A tool for de novo drug design. J Mol Graph 14:272-276 (1996)

Galvez J, Garcia-Domenech R, de Julian-Ortiz V, Soler R. Topological approach to analgesia. J Chem Inf Comput Sci 34:1198-1203 (1994)

Galvez J, Garcia-Domenech R, de Julian-Ortiz V, Soler R. Topological approach to drug design. J Chem Inf Comput Sci 35:272-284 (1995)

Galvez J, Gomez-Lechon M J, Garcia-Domenech R, Castell J V. New cytostatic agents obtained by molecular topology. Bioorg & Med Chem Lett 6:2301-2306 (1996)

Galvez J. On a topological interpretation of electronic and vibrational molecular energies. J Mol Struct 429:255-264 (1998)

Galvez J. Prediction of molecular volume and surface of alkanes by molecular topology. J Chem Inf Comput Sci 43:1231-9 (2003)

Garcia-Domenech R, de Julian-Ortiz J V, Duart M J, Garcia-Torrecillas J M, Anton-Fos G M, Rios_santamarina I, De Gregorio-Alapont C, Galvez J. Search of a topological pattern to evaluate toxicity of heterogeneous compounds. SAR QSAR Environ Res. 12:237-54 (2001)

Garcia-Garcia A, Galvez J, de Julian-Ortiz J V, Gracia-Domenech R, Munoz C, Guna R, Borras R. New agents active against Mycobacterium avium complex selected by molecular topology: a virtual screening method. JAC 53:65-73 (2004)

Gohlke P, Weiss S, Jansen A, Wienen W, Stangier J, Rascher W, Culman J, Unger T. AT1 receptor antagonist telmisartan administered peripherally inhibits central responses to angiotensin II in conscious rats. JPET 298:62-70 (2001)

Golde T E, Eckman C B, Younkin S G. Biochemical detection of Aβ isoforms: implications for pathogenesis, diagnosis, and treatment of Alzheimer's disease. Biochim. Biophys. Acta 1502:172-187 (2000)

Gozalbes R, Brun-Pascaud M, Garcia-Domenech R, Galvez J, Girard P M, Doucet J P, Derouin F. Prediction of quinolone activity against Mycobacterium avium by molecular topology and virtual computational screening. Antimicrob Agents Chemother 44:2764-2770 (2000)

Guo Z, Fratiglioni L, Zhu L, Fastborn J, Winblad B, Viitanen M. Occurrence and progression of dementia in a community population aged 75 years and older: relationship of antihypertensive medication use. Arch Neurol 56:991-6 (1999)

Hassler O. Vascular changes in senile brains. Acta Neuropathol 5:40-53 (1965)

Ho L, Qin W, Pompl P N, Xiang Z, Wang J, Zhao Z, Peng Y, Cambareri G, Rocher A, Mobbs C V, Hof P R and Pasinetti G M. Diet—induced insulin resistance promotes amyloidosis in a transgenic mouse model of Alzheimer's disease. FASEB J 18:902-4 (2004)

Hof P R, Bussiere T, Gold G, Kovari E, Giannakopoulos P, Bouras C, Perl D P, Morrison J H. Stereologic evidence for persistence of viable neurons in layer II of the entorhinal cortex and the CA1 field in Alzheimer disease. J Neuropathol Exp Neurol 62:55-67 (2003)

Howlett D R, George A R, Owen D E, Ward R V, Markwell R E. Common structural features determine the effectiveness of carvedilol, daunomycin and rolitetracycline as inhibitors of Alzheimer beta-amyloid fibril formation. Biochem J 343:419-23 (1999)

Hsiao K, Chapman P, Nilsen S, Eckman C, Harigaya Y, Younkin S, Yang F, Cole G. Correlative memory deficits. Aβelevation, and amyloid plaques in transgenic mice. Science 274:99-102 (1996)

Iijima K, Liu H P, Chiang A S, Heam S A, Konsolaki M, Zhong Y. Dissecting the pathological effects of human Abeta40 and Abeta42 in Drosophila: a potential model for Alzheimer's disease. Proc. Natl. Acad. Sci. USA 101:6623-6628 (2004)

in't Veld B A, Ruitenberg A, Hofman A, Stricker B H, Breteler M M. antihypertensive drugs and incidence of dementia: the Rotterdam study. Neurobiol Aging 22:407-12 (2001)

Israili Z H. Clinical pharmacokinetics of angiotensin II (AT1) receptor blockers in hypertension. J Hum Hypertens 14:S73-86 (2000)

Kawarabayashi T, Younkin L H, Saido T C, Shoji M, Ashe K H, Younkin S G. Age-dependent changes in brain, CSF, and plasma amyloid (beta) protein in the Tg2576 transgenic mouse model of Alzheimer's disease. J Neurosci 21:372-81 (2001)

Kivipelto M, Helkala E L, Laakso M P, Hanninen T, Hallikainen M, Alhainen K, Soinien H, Tuomilehto J, Nissinen A. Midlife vascular risk factors and Alzheimer's disease in later life: longitudinal, population based study. Br Med J 322:1447-1451 (2001)

Kocisko A D, Baron G S, Rubenstein R, Chen J, Kuizon S, Caughey B. New Inhibitors of scrapie-associated prion protein formation in a library of 2,000 drugs and natural products. J Virology 77:10288-10294 (2003)

Korf E S C, White L R, Scheltens P, Launer U. Midlife blood pressure and the risk of hippocampal atrophy, The Honolulu Asia aging study. Hypertension 44:29-34 (2004)

Kukar T, Murphy M P, Eriksen J L, Sagi S A, Weggen S, Smith T E, Ladd T, Khan M A, Kache R, Beard J, Dodson M, Merit S, Ozols V V, Anastasiadis P Z, Das P, Fauq A, Koo E H, Golde T E. Diverse compounds mimic Alzheimer's disease-causing mutations by augmenting Aβ42 production Nat Med 11:545-50 (2005)

Lahuerta Zamora L, Mestre F, Duart M J, Anton Fos G M, Garcia-Domenech R, Galvez Alvarez J, Martinez Calatayud J. Prediction of the Chemiluminescent behaviour of pharmaceuticals and pesticides. Anal Chem 73:4301-4306 (2001)

Langub M C Jr, Watson R E Jr, Herman J P. Distribution of natriuretic peptide precursor mRNAs in the rat brain. J Comp Neurol 356:183-99 (1995)

Launer L J, Ross G W, Petrovitc H, Masaki K, Foley D, White L R, Havlik R J. Midlife blood pressure and dementia: the Honolulu-Asia aging study. Neurobiol Aging 21:49-55 (2000)

Lee J Y, Friedman J E, angel I, Kozak A, Koh J Y. The lipophilic metal chelator DP-109 reduces amyloid pathology in brains of human beta-amyloid precursor protein transgenic mice. Neurobiol Aging 25:1315-21 (2004)

Levine T B, Levine A B. Clinical update: the role of angiotensin II receptor blockers in patients with left ventricular dysfunction (Part II of II). Clin Cardiol 28:277-80 (2005)

Lithell H, Hansson L, Skoog I, Elmfeldt D, Hofman A, Olofsson B, Trenkwalder P, Zanchetti A. The study on cognition and prognosis in the elderly (SCOPE): principal results of a randomized double-blind intervention trial. J Hypertension 21:875-886 (2003)

Liu X, Smith B J, Chen C, Callegari E, Becker S L, Chen X, Cianfrogna J, Doran A C, Doran S D, Gibbs J P, Hosea N, Liu J, Nelson F R, Szewc M A, Van Deusen J. Use of a physiologically based pharmacokinetic model to study the time to reach brain equilibrium: an experimental analysis of the role of blood-brain barrier permeability, plasma protein binding, and brain tissue binding. JPET 313:1254-1262 (2005)

Lopez-Arrieta J M, Birsks J. Nimodipine for primary degenerative, mixed and vascular dementia. Cochrane Database Syst Rev 3:CD000147 (2002)

Luchsinger J A, Mayeux R. Cardiovascular risk factors and Alzheimer's disease. Curr Atheroscler Rep 2004 6:261-6 (2004)

Luco J M, Galvez J, Garcia-Domenech R, de Julian-Ortiz J V. Structural invariants for the prediction of relative toxicities of polychloro dibenzo-p-dioxins and dibenzofurans. Mol Diversity 8:331-342 (2004)

McGowan, E. et al. Selective Overexpression of Aβ42, but not Aβ40, in the secretory pathway is sufficient for plaque deposition in mice. Abstr. Soc. Neurosci. 877:14 (2003)

Mirjany M, Ho L. and Pasinetti G. M., Role of cyclooxygenase-2 in neuronal cell cycle activity and glutamate mediated excitotoxicity. J Pharmacol. Exp. Therap. 301:494-500 (2002)

Ogihara T, Saruta T, Matsuoka H, Shimamoto K, Fujita T, Shimada K, Imai Y, Nishigaki M. Valsartan in elderly isolated systolic hypertension (VALISH) study: rationale and design. Hepertens Res 27:657-61 (2004)

Pangalos M N, Jacobsen S J, Reinhart P H. Disease modifying strategies for the treatment of Alzheimer's disease targeted at modulating levels of the beta-amyloid peptide. Biochem Soc Trans. 33:553-8 (2005)

Petrovitch H, White L R, Izmirilian G, Ross G W, Havlik R J, Markesbery W, Nelson J, Davis D G, Hardman Foley D J, Launer L J. Midlife blood pressure and neuritic plaques, neurofibrillary tangles, and brain weight at death: the HAAS. Honolulu-Asia aging Study. Neurobiol Aging 21:57-62 (2000)

Pompl P N, Ho L, Bianchi M, McManus T, Qin W, Pasinetti G M. A therapeutic role for cyclooxygenase-2 inhibitors in a transgenic mouse model of amyotrophic lateral sclerosis. FASEB J 17:725-727 (2003)

Religa D, Winblad B. Therapeutic strategies for Alzheimer's based on new molecular mechanisms. Acta Neurobiol Exp (Wars) 63:393-6 (2003)

Rios-Santamarina Igarcia-Domenech R, Galvez J, Morcillo E J, Santamaria P, Cortjo J. Getteing new bronchodilator compounds from molecular topology. Eur J Pharm Sci. 22:271-7 (2004)

Ripley T L. Valsartan in chronic heart failure. Ann Pharmacother 39:460-9 (2005)

Rodriguez A, Ehlenberger D, Kelliher K, Einstein M, Henderson S C, Morrison J H, Hof P R, Wearne S L. Automated reconstruction of a three-dimensional neuronal morphology from laser scanning microscopy images. Methods 30:94-105 (2003)

Roman G C. Vascular dementia prevention: a risk factor analysis. Cerebrovasc Dis 20:91-100 (2005)

Rothstein J, Patel S, Regan M R, Haenggeli C, Huang Y H, Bergles D E, Jin L, Dykes Hoberg M, Vidensky S, Chung D S, Toan S V, Bruijin L I, Su Z Z, Gupta P, Fisher P B. B-lactam antibiotics offer neuroprotection by increasing glutamate transporter expression. Lett Nat 433:73-77 (2005)

Rozzini L, Vicini Chilovi B, Bellelli G, Bertoletti E, Trabuchi M, Padovani A. Effects of cholinesterase inhibitors appear greater in patients on established antihypertensive therapy. Int J Geriatr Psychiatry 20:547-551 (2005)

Sagi S A, Weggen S, Eriksen J, Golde T E, Koo E H. The non-cyclooxygenase targets of non-steroidal anti-inflammatory drugs, lipoxygenases, peroxisome proliferator-activated receptor, inhibitor of kappa B kinase, and NF kappa B, do not reduce amyloid beta 42 production. J. Biol. Chem. 278:31825-31830 (2003)

Sano M. Current concepts in the prevention of Alzheimer's disease. CNS Spectr. 8:846-853 (2003)

Savaskan E, Hock C, Olivieri G, Bruttel S, Rosenberg C, Hulette C, Muller-Spahn F. Cortical alterations of angiotensin converting enzyme, angiotensin II and AT1 receptor in Alzheimer's dementia. Neurobiol Aging 22:541-6 (2001)

Scheuner D, Eckman C, Jensen M, Song X, Citron M, Suzuki N, Bird T D, Hardy J, Hutton M, Kukull W, Larson E, Levy-Lahad L, Viitanen M, Peskind E, Poorkaj P, Schellenberg G, Tanzi R, Wasco W, Lannfelt L, Selkoe D, Younkin S. Secreted amyloid beta-protein similar to that in the senile plaques of Alzheimer's disease is increased in vivo by the presenilin 1 and 2 and APP mutations linked to familial Alzheimer's disease. Nat. Med. 2:864-870 (1996)

Selkoe D J. Alzheimer's disease: genes, proteins, and therapy. Physiol. Rev. 81:741-766 (2001)

Shimbo D, Osende J, Chen J, Robbins J, Shimoto Y, Kunitada S, Fuster V, Badimon J J. Antithrombotic effects of DX-9065a, a direct factor Xa inhibitor: a comparative study in humans versus low molecular weight heparin. Thromb Haemost 88:733-8 (2002)

Shimohama S, Taniguchi T, Fujiwara M, Kameyama M. Biochemical characterization of alpha-adrenergic receptors in human brain and changes in Alzheimer-type dementia. J Neurochem 47:1295-301 (1986)

Sparks D L, Scheff S W, Liu H, Landers T M, Coyne C M, Hunsaker J C, III. Increased incidence of neurofibrillary tangels (NTF) in non-demented individuals with hypertension. J Neurol Sci 131:162-169 (1995)

Spence J D. Preventing dementia by treating hypertension and preventing stroke. Hypertension 44:20-21 (2004)

Staessen J A, Birkenhager W H. Cognitive impairnment and blood pressure. Quo usque tandem abutere patientia Nostra? Hypertension 44:612-613 (2004)

Tachikawa H, Kodama M, Hui L, Yoshida T, Hayashi M, Abe S, Kashimura T, Kato K, Hanawa H, Watanabe K, Nakazawa M, Aizawa Y. Angiotensin II type 1 receptor blocker, valsartan, prevented cardiac fibrosis in rat cardiomyopathy after autoimmune myocarditis. J Cardiovasc Pharmacol 41:S105-10 (2003)

The PROGRESS Collaborative Group. Effects of blood pressure lowering with perindopril and indapamide therapy on dementia and cognitive decline in patients with cerebrovascular disease. Arch Intern Med 163:1069-1075 (2003)

Unger T. Inhibiting angiotensin receptors in the brain: possible therapeutic implications. Curr Med Res Opin 19:449-451 (2003)

Wang J, Ho L, Qin W, Rocher A. B, Seror I, Humala N, Maniar K, Dolios G, Wang R, Hof P R, Pasinetti G M. Caloric restriction attenuates □-amyloid neuropathology in a mouse model of Alzheimer's disease. FASEB J 19:659-61 (2005)

Watson W P, Little H J. Selectivity of the protective effects of dihydropyridine calcium chanel antagonists against the ethanol withdrawal syndrome. Brain Res 930:111-22 (2002)

Wearne S L, Rodriguez A, Ehlenberger D B, Rocher A B, Henderson S C, Hof P R. New techniques for imaging, digitization and analysis of three-dimensional neural morphology on multiple scales. Neuroscience 136:661-680 (2005)

Weggen S, Eriksen J L, Das P, Sagi S A, Wang R, Pietrzik C U, Findlay K A, Smith T E, Murphy M P, Bulter T, Kang D E, Marquez-Sterling N, Golde T E, Koo E H. A subset of NSAIDs lower amyloidogenic Aβ42 independently of cyclooxygenase activity. Nature 414:212-216 (2001) p Weggen S, Eriksen J L, Sagi S A, Pietrzik C U, Ozols V, Fauq A, Golde T E, Koo E H. Evidence that nonsteroidal anti-inflammatory drugs decrease amyloid beta 42 production by direct modulation of gamma-secretase activity. J. Biol. Chem. 278:31831-31837 (2003)

Wu C, Zhou D, Wen C, zhang L, Como P, Qiao Y. Relationship between blood pressure and Alzheimer's disease in Linxian County, China. Life Sciences 72:1125-1133 (2003)

Yamamoto S, Hayashi N, Kometani M, Nakao K, Inukai T. Pharmacological profile of valsaratan, a non-peptide angiotensin II type 1 receptor antagonis. 1st communication: antihypertensive effects of valsartan in hypertensive models. Arneimittelforschung 47:604-12 (1997)

Yasunari K, Maeda K, Nakamura M, Watanabe T, Yoshikawa J, Hirohashi K. Left ventricular hypertrophy and angiotensin II receptor blocking agants. Curr Med Chem Cardiovasc Hematol Agents 3:61-7 (2005)

Younkin, S. G. The role of Aβ42 in Alzheimer's disease. J. Physiol. Paris 92:289-292 (1998)

Zhang F, Eckman C, Younkin S, Hsiao K K, ladecola C. Increased susceptibility to ischemic brain damage in transgenic mice overexpressing the amyloid precursor protein. J Neurosci 17:7655-61 (1997)

Zhao Z, Ho L., Wang J, Qin W, Festa E D, Mobbs C, Hof P, Rocher A, Masur S, Haroutunian V, Pasinetti G M. Connective tissue growth factor (CTGF) expression in the brain is a downstream effector of insulin resistance-associated promotion of Alzheimer's disease beta-amyloid neuropathology. FASEB J. 19:2981-2 (2005)

What is claimed is:

1. A method of reducing Aβ1-40 generation in primary cortico-hippocampal neurons of a mammal comprising administering to said mammal a composition comprising an cardiovascular agent selected from the group consisting of Metergoline; Suloctidil; Bumetanide; Ethacrynic Acid; Tetrandrine; Perhexiline Maleate; Amlodipine Besylate; Bepridil Hydrochloride; Prazosin Hydrochloride; Fendiline Hydrochloride; Candesartan Cilextil; Nicardipine Hydrochloride; Fenofibrate; Amiodarone Hydrochloride; Papaverine Hydrochloride; N,N-Hexamethyleneamiloride; Reserpine; Simvastatin; Cadmium Acetate; Nitrendipine; Propafenone Hydrochloride; Carvedilol; Flunarizine Hydrochloride; Oxidopamine Hydrochloride; Lanatoside C; Lanatoside C; Dicumarol; Valsartan; Propranolol Hydrochloride (−); Veratrine Sulfate; Vinpocetine; Spironolactone; Protoveratrine B; Quinidine Gluconate; Propranolol Hydrochloride (±); Atorvastatin Calcium; Hydralazine Hydrochloride; Yohimbine Hydrochloride; Xylometazoline Hydrochloride; Digitoxin; Nylidrin Hydrochloride; Verapamil; Cyclothiazide; Chrysin; Scopoletin; Dipyridamole; Nifedipine; Althiazide; Losartan; Nicergoline; Bendrofumethiazide; Probucol; Amiloride Hydrochloride; Oxymetazoline Hydrochloride; Isoxsuprine Hydrochloride; Isoxsuprine Hydrochloride; Pargyline Hydrochloride; Nimodipine; Neriifolin; Nicotinyl Tartrate; Isosorbide Dinitrate; Pempidine Tartrate; 2-(2,6-Dimethoxyphenoxyethyl); Aminomethyl-1,4-Benzodioxane; Hydrochloride; Phentolamine Hydrochloride; Disopyramide Phosphate; Rosuvastatin; Perindopril Erbumine; Olmesartan Medoxomil; Hexamethonium Bromide; Labetalol Hydrochloride; Tranexamic Acid; and Dopamine Hydrochloride; analogs thereof and combinations thereof.

2. A method of reducing Aβ1-42 generation in primary cortico-hippocampal neurons of a mammal comprising administering to said mammal a composition comprising an cardiovascular agent selected from the group consisting of Ethacrynic Acid; Metergoline; Cadmium Acetate; Suloctidil; Amlodipine Besylate; Candesartan Cilextil; Bepridil Hydrochloride; Prazosin Hydrochloride; Amiodarone Hydrochloride; Tetrandrine; Perhexiline Maleate; Fendiline Hydrochloride; N,N-Hexamethyleneamiloride; Nicardipine Hydrochloride; Papaverine Hydrochloride; Carvedilol; Propranolol Hydrochloride (−); Oxidopamine Hydrochloride; Reserpine; Valsartan; Oxymetazoline Hydrochloride; Pindolol; Amiloride Hydrochloride; Flunarizine Hydrochloride; Tranexamic Acid; Dicumarol; Propafenone Hydrochloride; Bendrofumethiazide; Dipyridamole; Hydralazine Hydrochloride; Nitrendipine; Triamterene; Althiazide; Rosuvastatin; Disopyramide Phosphate; Isosorbide Dinitrate; Alfluzosin; Yohimbine Hydrochloride; Bucladesine; Quinidine Gluconate; Spironolactone; Olmesartan Medoxomil; Xylometazoline Hydrochloride; Hexamethonium Bromide; Phentolamine Hydrochloride; Nicotinyl Tartrate; Rauwolscine Hydrochloride; Bumetanide; Cyclothiazide; Midodrine Hydrochloride; Atorvastatin Calcium; Fenofibrate; Dopamine Hydrochloride; Pempidine Tartrate; Fenoterol Hydrobromide; Irbesartan; Chrysin; Isoxsuprine Hydrochloride; Isoxsuprine Hydrochloride; and Trichlormethiazide and analogs thereof and combinations thereof.

3. A method of treating Alzheimer's disease in a mammal comprising administering to said mammal a composition comprising an cardiovascular agent agent selected from the group consisting of Metergoline; Suloctidil; Bumetanide; Ethacrynic Acid; Tetrandrine; Perhexiline Maleate; Amlodipine Besylate; Bepridil Hydrochloride; Prazosin Hydrochloride; Fendiline Hydrochloride; Candesartan Cilextil; Nicardipine Hydrochloride; Fenofibrate; Amiodarone Hydrochloride; Papaverine Hydrochloride; N,N-Hexamethyleneamiloride; Reserpine; Simvastatin; Cadmium Acetate; Nitrendipine; Propafenone Hydrochloride; Carvedilol; Flunarizine Hydrochloride; Oxidopamine Hydrochloride; Lanatoside C; Lanatoside C; Dicumarol; Valsartan; Propranolol Hydrochloride (−); Veratrine Sulfate; Vinpocetine; Spironolactone; Protoveratrine B; Quinidine Gluconate; Propranolol Hydrochloride (±); Atorvastatin Calcium; Hydralazine Hydrochloride; Yohimbine Hydrochloride; Xylometazoline Hydrochloride; Digitoxin; Nylidrin Hydrochloride; Verapamil; Cyclothiazide; Chrysin; Scopoletin; Dipyridamole; Nifedipine; Althiazide; Losartan; Nicergoline; Bendrofumethiazide; Probucol; Amiloride Hydrochloride; Oxymetazoline Hydrochloride; Isoxsuprine Hydrochloride; Isoxsuprine Hydrochloride; Pargyline Hydrochloride; Nimodipine; Neriifolin; Nicotinyl Tartrate; Isosorbide Dinitrate; Pempidine Tartrate; 2-(2,6-Dimethoxyphenoxyethyl); Aminomethyl-1,4-Benzodioxane; Hydrochloride; Phentolamine Hydrochloride; Disopyramide Phosphate; Rosuvastatin; Perindopril Erbumine; Olmesartan Medoxomil; Hexamethonium Bromide; Labetalol Hydrochloride; Tranexamic Acid; and Dopamine Hydrochloride; analogs thereof and combinations thereof, in an amount effective to ameliorate at least one symptom of said disease in said mammal.

4. A method of treating Alzheimer's disease in a mammal comprising administering to said mammal a composition comprising an cardiovascular agent agent selected from the group consisting of Ethacrynic Acid; Metergoline; Cadmium Acetate; Suloctidil; Amlodipine Besylate; Candesartan Cilextil; Bepridil Hydrochloride; Prazosin Hydrochloride; Amiodarone Hydrochloride; Tetrandrine; Perhexiline Maleate; Fendiline Hydrochloride; N,N-Hexamethyleneamiloride; Nicardipine Hydrochloride; Papaverine Hydrochloride; Carvedilol; Propranolol Hydrochloride (−); Oxidopamine Hydrochloride; Reserpine; Valsartan; Oxymetazoline Hydrochloride; Pindolol; Amiloride Hydrochloride; Flunarizine Hydrochloride; Tranexamic Acid; Dicumarol; Propafenone Hydrochloride; Bendrofumethiazide; Dipyridamole; Hydralazine Hydrochloride; Nitrendipine; Triamterene; Althiazide; Rosuvastatin; Disopyramide Phosphate; Isosorbide Dinitrate; Alfluzosin; Yohimbine Hydrochloride; Bucladesine; Quinidine Gluconate; Spironolactone; Olmesartan Medoxomil; Xylometazoline Hydrochloride; Hexamethonium Bromide; Phentolamine Hydrochloride; Nicotinyl Tartrate; Rauwolscine Hydrochloride; Bumetanide; Cyclothiazide; Midodrine Hydrochloride; Atorvastatin Calcium; Fenofibrate; Dopamine Hydrochloride; Pempidine Tartrate; Fenoterol Hydrobromide; Irbesartan; Chrysin; Isoxsuprine Hydrochloride; Isoxsuprine Hydrochloride; and Trichlormethiazide and analogs thereof and combinations thereof.

5. The method of claim 3 or 4, wherein said administration of said cardiovascular agent to said animal decreases Aβ generation in the brain of said mammal to decrease or prevent the likelihood of AD amyloid neuropathy in said mammal.

6. The method of claim 3 or claim 4, wherein said administration of said cardiovascular agent to said animal increase Aβ clearance from the brain, to decrease or prevent the likelihood of AD amyloid neuropathy in said mammal.

7. The method of claim 3 or claim 4, wherein said administration of said cardiovascular agent to said animal decreases cognitive deterioration in the mammal as compared to the cognitive deterioration of a mammal with AD in the absence of said administration of said cardiovascular agent.

8. The method of claim 3 or claim 4, wherein the treatment is determined by the improvement, or reduction or arrest of deterioration in at least one of the assessments selected from the group consisting of the Alzheimer's Disease Assessment Scale-cognitive subscale (ADAS-cog), the Alzheimer's Disease Cooperative Study-Activities of Daily Living (ADCS-ADL) Inventory and Clinician's Interview-Based Impression of Change Plus Version (CIBIC-plus).

9. The method of any of claims 1 to 8 wherein said administration of said cardiovascular agent to said animal increase Aβ clearance from the brain, to decrease or prevent the likelihood of AD amyloid neuropathy in said mammal.

10. The method of any of claims 1 to 9 wherein the dose of cardiovascular agent used is at least 2-fold less than the dose of said agent recommended used for use in hypertension.

11. The method of any of claims 1 through 10 wherein said administration said cardiovascular agent reduces the ratio of Aβ1-42 to Aβ1-40 as % value as compared to control mammals that do not receive the cardiovascular agent.

12. The method of claims 1 through 11 wherein said method produces a reduction in the amount of HMW Aβ oligomer formation in the cerebral cortex of said mammal.

13. Use of an cardiovascular agent selected from the group consisting of Ethacrynic Acid; Metergoline; Cadmium Acetate; Suloctidil; Amlodipine Besylate; Candesartan Cilextil; Bepridil Hydrochloride; Prazosin Hydrochloride; Amiodarone Hydrochloride; Tetrandrine; Perhexiline Maleate; Fendiline Hydrochloride; N,N-Hexamethyleneamiloride; Nicardipine Hydrochloride; Papaverine Hydrochloride; Carvedilol; Propranolol Hydrochloride (−); Oxidopamine Hydrochloride; Reserpine; Valsartan; Oxymetazoline Hydrochloride; Pindolol; Amiloride Hydrochloride; Flunarizine Hydrochloride; Tranexamic Acid; Dicumarol; Propafenone Hydrochloride; Bendrofumethiazide; Dipyridamole; Hydralazine Hydrochloride; Nitrendipine; Triamterene; Althiazide; Rosuvastatin; Disopyramide Phosphate; Isosorbide Dinitrate; Alfluzosin; Yohimbine Hydrochloride; Bucladesine; Quinidine Gluconate; Spironolactone; Olmesartan Medoxomil; Xylometazoline Hydrochloride; Hexamethonium Bromide; Phentolamine Hydrochloride; Nicotinyl Tartrate; Rauwolscine Hydrochloride; Bumetanide; Cyclothiazide; Midodrine Hydrochloride; Atorvastatin Calcium; Fenofibrate; Dopamine Hydrochloride; Pempidine Tartrate; Fenoterol Hydrobromide; Irbesartan; Chrysin; Isoxsuprine Hydrochloride; Isoxsuprine Hydrochloride; and Trichlormethiazide and analogs and combinations thereof for the manufacture of a medicament for the treatment of Alzheimer's Disease.

14. Use of an cardiovascular agent selected from the group consisting of Ethacrynic Acid; Metergoline; Cadmium Acetate; Suloctidil; Amlodipine Besylate; Candesartan Cilextil; Bepridil Hydrochloride; Prazosin Hydrochloride; Amiodarone Hydrochloride; Tetrandrine; Perhexiline Maleate; Fendiline Hydrochloride; N,N-Hexamethyleneamiloride; Nicardipine Hydrochloride; Papaverine Hydrochloride; Carvedilol; Propranolol Hydrochloride (−); Oxidopamine Hydrochloride; Reserpine; Valsartan; Oxymetazoline Hydrochloride; Pindolol; Amiloride Hydrochloride; Flunarizine Hydrochloride; Tranexamic Acid; Dicumarol; Propafenone Hydrochloride; Bendrofumethiazide; Dipyridamole; Hydralazine Hydrochloride; Nitrendipine; Triamterene; Althiazide; Rosuvastatin; Disopyramide Phosphate; Isosorbide Dinitrate; Alfluzosin; Yohimbine Hydrochloride; Bucladesine; Quinidine Gluconate; Spironolactone; Olmesartan Medoxomil; Xylometazoline Hydrochloride; Hexamethonium Bromide; Phentolamine Hydrochloride; Nicotinyl Tartrate; Rauwolscine Hydrochloride; Bumetanide; Cyclothiazide; Midodrine Hydrochloride; Atorvastatin Calcium; Fenofibrate; Dopamine Hydrochloride; Pempidine Tartrate; Fenoterol Hydrobromide; Irbesartan; Chrysin; Isoxsuprine Hydrochloride; Isoxsuprine Hydrochloride; and Trichlormethiazide and analogs and combinations for the treatment of Alzheimer's Disease.

15. Use of an cardiovascular agent selected from the group consisting of Metergoline; Suloctidil; Bumetanide; Ethacrynic Acid; Tetrandrine; Perhexiline Maleate; Amlodipine Besylate; Bepridil Hydrochloride; Prazosin Hydrochloride; Fendiline Hydrochloride; Candesartan Cilextil; Nicardipine Hydrochloride; Fenofibrate; Amiodarone Hydrochloride; Papaverine Hydrochloride; N,N-Hexamethyleneamiloride; Reserpine; Simvastatin; Cadmium Acetate; Nitrendipine; Propafenone Hydrochloride; Carvedilol; Flunarizine Hydrochloride; Oxidopamine Hydrochloride; Lanatoside C; Lanatoside C; Dicumarol; Valsartan; Propranolol Hydrochloride (−); Veratrine Sulfate; Vinpocetine; Spironolactone; Protoveratrine B; Quinidine Gluconate; Propranolol Hydrochloride (±); Atorvastatin Calcium; Hydralazine Hydrochloride; Yohimbine Hydrochloride; Xylometazoline Hydrochloride; Digitoxin; Nylidrin Hydrochloride; Verapamil; Cyclothiazide; Chrysin; Scopoletin; Dipyridamole; Nifedipine; Althiazide; Losartan; Nicergoline; Bendrofumethiazide; Probucol; Amiloride Hydrochloride; Oxymetazoline Hydrochloride; Isoxsuprine Hydrochloride; Isoxsuprine Hydrochloride; Pargyline Hydrochloride; Nimodipine; Neriifolin; Nicotinyl Tartrate; Isosorbide Dinitrate; Pempidine Tartrate; 2-(2,6-Dimethoxyphenoxyethyl); Aminomethyl-1,4-Benzodioxane; Hydrochloride; Phentolamine Hydrochloride; Disopyramide Phosphate; Rosuvastatin; Perindopril Erbumine; Olmesartan Medoxomil; Hexamethonium Bromide; Labetalol Hydrochloride; Tranexamic Acid; and Dopamine Hydrochloride; analogs thereof and combinations thereof for the manufacture of a medicament for the treatment of Alzheimer's Disease.

16. Use of an cardiovascular agent selected from the group consisting of Metergoline; Suloctidil; Bumetanide; Ethacrynic Acid; Tetrandrine; Perhexiline Maleate; Amlodipine Besylate; Bepridil Hydrochloride; Prazosin Hydrochloride; Fendiline Hydrochloride; Candesartan Cilextil; Nicardipine Hydrochloride; Fenofibrate; Amiodarone Hydrochloride; Papaverine Hydrochloride; N,N-Hexamethyleneamiloride; Reserpine; Simvastatin; Cadmium Acetate; Nitrendipine; Propafenone Hydrochloride; Carvedilol; Flunarizine Hydrochloride; Oxidopamine Hydrochloride; Lanatoside C; Lanatoside C; Dicumarol; Valsartan; Propranolol Hydrochloride (−); Veratrine Sulfate; Vinpocetine; Spironolactone; Protoveratrine B; Quinidine Gluconate; Propranolol Hydrochloride (±); Atorvastatin Calcium; Hydralazine Hydrochloride; Yohimbine Hydrochloride; Xylometazoline Hydrochloride; Digitoxin; Nylidrin Hydrochloride; Verapamil; Cyclothiazide; Chrysin; Scopoletin; Dipyridamole; Nifedipine; Althiazide; Losartan; Nicergoline; Bendrofumethiazide; Probucol; Amiloride Hydrochloride; Oxymetazoline Hydrochloride; Isoxsuprine Hydrochloride; Isoxsuprine Hydrochloride; Pargyline Hydrochloride; Nimodipine; Neriifolin; Nicotinyl Tartrate; Isosorbide Dinitrate; Pempidine Tartrate; 2-(2,6-Dimethoxyphenoxyethyl); Aminomethyl-1,4-Benzodioxane; Hydrochloride; Phentolamine Hydrochloride; Disopyramide Phosphate; Rosuvastatin; Perindopril Erbumine; Olmesartan Medoxomil; Hexamethonium Bromide; Labetalol Hydrochloride; Tranexamic Acid; and Dopamine Hydrochloride; analogs thereof and combinations thereof for the treatment of Alzheimer's Disease.

17. Use of any of claims 13 or 16, wherein said administration of said cardiovascular agent to said animal decreases Aβ generation in the brain of said mammal to decrease or prevent the likelihood of AD amyloid neuropathy in said mammal.

18. The method of claim 13 or claim 17, wherein said administration of said cardiovascular agent to said animal increase Aβ clearance from the brain, to decrease or prevent the likelihood of AD amyloid neuropathy in said mammal.

19. The method of claim 13 or claim 18, wherein said administration of said cardiovascular agent to said animal decreases cognitive deterioration in the mammal as compared to the cognitive deterioration of a mammal with AD in the absence of said administration of said cardiovascular agent.

20. The method of claim 13 or claim 19, wherein the treatment is determined by the improvement, or reduction or arrest of deterioration in at least one of the assessments selected from the group consisting of the Alzheimer's Disease Assessment Scale-cognitive subscale (ADAS-cog), the Alzheimer's Disease Cooperative Study-Activities of Daily Living (ADCS-ADL) Inventory and Clinician's Interview-Based Impression of Change Plus Version (CIBIC-plus).

21. The method of any of claims 13 to 20 wherein said administration of said cardiovascular agent to said animal increase Aβ clearance from the brain, to decrease or prevent the likelihood of AD amyloid neuropathy in said mammal.

22. The method of any of claims 13 to 21 wherein the dose of cardiovascular agent used is at least 2-fold less than the dose of said agent recommended used for use in hypertension.

23. The method of any of claims 13 through 22 wherein said administration said cardiovascular agent reduces the ratio of Aβ1-42 to Aβ1-40 as % value as compared to control mammals that do not receive the cardiovascular agent.

24. The method of claims 13 through 23 wherein said method produces a reduction in the amount of HMW Aβ oligomer formation in the cerebral cortex of said mammal.
